(19) United States
(12) Patent Application Publication
(10) Pub. No.: US 2011/0288059 A1
(43) Pub. Date: Nov. 24, 2011

(54) NEUROACTIVE STEROID COMPOSITIONS AND METHODS OF USE THEREFOR

(76) Inventors: Christine E. Marx, Durham, NC (US); Jed E. Rose, Durham, NC (US)

(21) Appl. No.: 13/191,140

(22) Filed: Jul. 26, 2011

Related U.S. Application Data
(62) Division of application No. 12/008,259, filed on Jan. 8, 2008, now abandoned.
(60) Provisional application No. 60/879,165, filed on Jan. 8, 2007.

(51) Int. Cl. A61K 31/57; A61P 25/00; A61P 25/08; A61P 25/22; A61P 25/18; A61K 31/56; A61P 25/24

(52) U.S. Cl. 514/170; 514/182; 514/171

(57) ABSTRACT

Provided are methods for ameliorating a symptom of a neuropsychiatric disorder in a subject. Also provided are methods for ameliorating at least one physical symptom or at least one psychological symptom resulting from tobacco cessation in a subject, methods for ameliorating a symptom of Alzheimer's disease or other cognitive disorder in a subject, methods for ameliorating a symptom of schizophrenia, schizoaffective disorder, or other psychotic disorder in a subject, methods for ameliorating a symptom of a depressive disorder in a subject, methods for ameliorating a symptom of bipolar disorder in a subject, methods for ameliorating a symptom of post-traumatic stress disorder or other anxiety disorder in a subject, methods for predicting a predisposition to suicide, suicidal ideation, suicidal behavior, or a combination thereof in a subject, methods for ameliorating a symptom of a pain disorder in a subject, methods for ameliorating a neurodegenerative disorder in a subject, methods for ameliorating a symptom of traumatic brain injury in a subject, methods for ameliorating a sleep disorder in a subject, and methods for improving cognitive functioning in a subject. In some embodiments, the methods include administering to a subject in need thereof an effective amount of a neuroactive steroid composition comprising pregnenolone (PG), allopregnanolone (ALLO), dehydroepiandrosterone (DHEA), pharmaceutically acceptable salts thereof, derivatives thereof, or combinations thereof.

### RELATED APPLICATIONS

This application is a divisional of U.S. patent application Ser. No. 12/008,259, filed Jan. 8, 2008, which itself claims the benefit of U.S. Provisional Patent Application Ser. No. 60/879,165; filed Jan. 8, 2007. The disclosure of each of these applications is incorporated herein by reference in its entirety.

### GOVERNMENT INTEREST

This presently disclosed subject matter was made with U.S. Government support under Grant Nos. MH 65080, MH 70448, and AG05128 awarded by the National Institutes of Health. Additional support came from a Veterans Affairs (VA) Advanced Research Career Development Award and VA Mid-Atlantic Mental Illness, Research, Education, and Clinical Center (MIRECC) award from the VA. Thus, the U.S. Government has certain rights in the presently disclosed subject matter.

### TECHNICAL FIELD

The presently disclosed subject matter relates to methods for treating neurological and/or psychiatric disorders and/or ameliorating one or more symptoms thereof, comprising administering to a subject in need thereof an effective amount of one or more neuroactive steroids, pharmaceutically acceptable salts thereof, derivatives thereof, or combinations thereof. Also provided are methods for prophylaxis comprising administering to a subject in need thereof one of the presently disclosed neuroactive steroids, pharmaceutically acceptable salts thereof, derivatives thereof, or combinations thereof.

### BACKGROUND

Mental health disorders broadly construed affect tens of millions of Americans, and many millions of others around the world. The cost of diagnosing and treating these subjects runs in the billions of dollars each year. Even with newer therapies, treatment for many mental health disorders remains intractable and is compromised by therapeutics that are frequently inadequate, and/or have other negative side effects, and/or are characterized by the development of dependence and/or tolerance. These frequent drawbacks limit the therapeutics that can be efficaciously and/or safely given to subjects in need.

Therefore, there exists a long-felt and ongoing need in the art for improved methods and new compositions for treating subjects with symptoms associated with neuropsychiatric disorders. Also urgently needed are new methods and compositions for treating subjects prophylactically who might be at risk for developing one or more symptoms typically associated with a neuropsychiatric disorder.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides methods for ameliorating a symptom of a neuropsychiatric disorder in a subject. In some embodiments, the methods comprise administering to the subject an effective amount of a neuroactive steroid composition comprising pregnenolone (PG), allopregnanolone (ALLO), dehydroepiandrosterone (DHEA), pharmaceutically acceptable salts thereof, derivatives thereof, or combinations thereof. In some embodiments, the neuroactive steroid composition is administered in a sustained release formulation, a controlled release formulation, or a combination thereof.

The presently disclosed subject matter also provides methods for ameliorating at least one physical symptom or at least one psychological symptom resulting from tobacco cessation in a subject. In some embodiments, the methods comprise administering to the subject an effective amount of a neuroactive steroid composition comprising pregnenolone (PG), allopregnanolone (ALLO), dehydroepiandrosterone (DHEA), pharmaceutically acceptable salts thereof, derivatives thereof, or combinations thereof. In some embodiments, the neuroactive steroid composition is administered in a sustained release formulation, a controlled release formulation, or a formulation for both sustained and controlled release.

The presently disclosed subject matter also provides methods for ameliorating a symptom of Alzheimer's disease or other cognitive disorder in a subject. In some embodiments, the methods comprise administering to the subject an effective amount of allopregnanolone (ALLO), pharmaceutically acceptable salts thereof, derivatives thereof, or combinations thereof. In some embodiments, the methods comprise administering to the subject an effective amount of pregnenolone (PG), pharmaceutically acceptable salts thereof, derivatives thereof, or combinations thereof.

The presently disclosed subject matter also provides methods for ameliorating a symptom of schizophrenia, schizoaffective disorder, or other psychotic disorder in a subject. In some embodiments, the methods comprise administering to the subject an effective amount of allopregnanolone (ALLO), pharmaceutically acceptable salts thereof, derivatives thereof, or combinations thereof. In some embodiments, the methods comprise administering to the subject an effective amount of pregnenolone (PG), pharmaceutically acceptable salts thereof, derivatives thereof, or combinations thereof.

The presently disclosed subject matter also provides methods for ameliorating a symptom of a depressive disorder (with or without psychotic features) or other mood disorder in a subject. In some embodiments, the methods comprise administering to the subject an effective amount of a neuroactive steroid composition comprising pregnenolone (PG), allopregnanolone (ALLO), dehydroepiandrosterone (DHEA), pharmaceutically acceptable salts thereof, derivatives thereof, or combinations thereof.

The presently disclosed subject matter also provides methods for ameliorating a symptom of bipolar disorder in a subject. In some embodiments, the methods comprise administering to the subject an effective amount of a neuroactive steroid composition comprising pregnenolone (PG), allopregnanolone (ALLO), dehydroepiandrosterone (DHEA), pharmaceutically acceptable salts thereof, derivatives thereof, or combinations thereof.

The presently disclosed subject matter also provides methods for ameliorating a symptom of post-traumatic stress disorder or other anxiety disorder in a subject. In some embodiments, the methods comprise administering to the subject an effective amount of a neuroactive steroid composition comprising pregnenolone (PG), allopregnanolone (ALLO), dehydroepiandrosterone (DHEA), pharmaceutically acceptable salts thereof, derivatives thereof, or combinations thereof.

The presently disclosed subject matter also provides methods for ameliorating a symptom of a pain disorder (e.g., a chronic pain disorder) in a subject. In some embodiments, the methods comprise administering to the subject an effective amount of a neuroactive steroid composition comprising pregnenolone (PG), allopregnanolone (ALLO), dehydroepiandrosterone (DHEA), pharmaceutically acceptable salts thereof, derivatives thereof, or combinations thereof.

The presently disclosed subject matter also provides methods for ameliorating a symptom of an alcohol use disorder or other substance use disorder in a subject. In some embodiments, the methods comprise administering to the subject an effective amount of a neuroactive steroid composition comprising pregnenolone (PG), allopregnanolone (ALLO), dehydroepiandrosterone (DHEA), pharmaceutically acceptable salts thereof, derivatives thereof, or combinations thereof.

The presently disclosed subject matter also provides methods for ameliorating a symptom of a sleep disorder in a subject. In some embodiments, the methods comprise administering to the subject an effective amount of a neuroactive steroid composition comprising pregnenolone (PG), allopregnanolone (ALLO), dehydroepiandrosterone (DHEA), pharmaceutically acceptable salts thereof, derivatives thereof, or combinations thereof.

The presently disclosed subject matter also provides methods for ameliorating a symptom of a seizure disorder in a subject. In some embodiments, the methods comprise administering to the subject an effective amount of a neuroactive steroid composition comprising pregnenolone (PG) or allopregnanolone (ALLO), dehydroepiandrosterone (DHEA), pharmaceutically acceptable salts thereof, derivatives thereof, or combinations thereof.

The presently disclosed subject matter also provides methods for ameliorating a symptom of a neurodegenerative disorder in a subject. In some embodiments, the methods comprise administering to the subject an effective amount of a neuroactive steroid composition comprising pregnenolone (PG), allopregnanolone (ALLO), dehydroepiandrosterone (DHEA), pharmaceutically acceptable salts thereof, derivatives thereof, or combinations thereof. In some embodiments, the neurodegenerative disorder is selected from the group consisting of multiple sclerosis, Parkinson's disease, and Niemann-Pick type C disease.

The presently disclosed subject matter also provides methods for ameliorating a symptom of traumatic brain injury and/or concussion in a subject. In some embodiments, the methods comprise administering to the subject an effective amount of a neuroactive steroid composition comprising pregnenolone (PG), allopregnanolone (ALLO), dehydroepiandrosterone (DHEA), pharmaceutically acceptable salts thereof, derivatives thereof, or combinations thereof.

The presently disclosed subject matter also provides methods for improving cognitive functioning in a subject. In some embodiments, the methods comprise administering to the subject an effective amount of a neuroactive steroid composition comprising pregnenolone (PG), allopregnanolone (ALLO), dehydroepiandrosterone (DHEA), pharmaceutically acceptable salts thereof, derivatives thereof, or combinations thereof. In some embodiments, the subject has schizophrenia, schizoaffective disorder, or other psychotic disorder, a depressive disorder (with or without psychotic features) or other mood disorder, PTSD or other anxiety disorder, alcohol use disorder or other substance use disorder, tobacco use disorder, a pain disorder, traumatic brain injury and/or concussion, attention deficit hyperactivity disorder, cognitive symptoms associated with menopause, a neurodegenerative disorder, or bipolar disorder.

The presently disclosed subject matter also provides methods for diagnosing Alzheimer's disease, monitoring the progression of Alzheimer's disease, and/or monitoring a response to an anti-Alzheimer's disease therapy in a subject. In some embodiments, the methods comprise detecting a change in a level of one or more neuroactive steroids in a biological sample from the subject and/or comparing a level of one or more neuroactive steroids in a biological sample from the subject to that in the same biological sample from a positive or a negative control subject, whereby Alzheimer's disease is diagnosed in the subject, the progression of Alzheimer's disease is monitored in the subject, and/or a response to an anti-Alzheimer's disease therapy is monitored in the subject.

The presently disclosed subject matter also provides methods for predicting a predisposition to suicide, suicidal ideation, suicidal behavior, or a combination thereof in a subject. In some embodiments, the methods comprise (a) determining a level of one or more neuroactive steroids in a sample isolated from the subject; and (b) comparing the level determined in step (a) to a standard, wherein the level of the one or more neuroactive steroids in the sample compared to the level in the standard is indicative of a predisposition to suicide, suicidal ideation, suicidal behavior, or combinations thereof. In some embodiments, the subject has a neuropsychiatric disorder selected from the group consisting of schizophrenia, schizoaffective disorder, or other psychotic disorder, Alzheimer's disease or other neurodegenerative disorder, Attention Deficit Disorder/Attention Deficit Hyperactivity Disorder, depressive disorder, bipolar disorder, post-traumatic stress disorder (PTSD) or other anxiety disorder, a pain disorder, tobacco dependence, alcohol abuse, alcohol dependence, drug dependence, drug abuse, traumatic brain injury and/or concussion, and combinations thereof. In some embodiments, the sample is selected from the group consisting of cerebrospinal fluid, serum, plasma, blood, saliva, skin, muscle, olfactory tissue, lacrimal fluid, synovial fluid, nail tissue, hair, feces, urine, a tissue or cell type, and combinations thereof. In some embodiments, the standard comprises a sample from a subject that does not have a predisposition to suicide, suicidal ideation, suicidal behavior, or combinations thereof.

The presently disclosed subject matter also provides methods for delaying or preventing the onset of, or decreasing the severity of, a symptom associated with a neuropsychiatric disorder in a subject in need thereof. In some embodiments, the presently disclosed methods comprise administering to the subject in need thereof an effective amount of a neuroactive steroid composition comprising pregnenolone (PG), allopregnanolone (ALLO), dehydroepiandrosterone (DHEA), pharmaceutically acceptable salts thereof, derivatives thereof, or combinations thereof. In some embodiments, the administering is performed prior to the subject in need thereof experiencing a condition expected to result in the symptom associated with a neuropsychiatric disorder developing and/or worsening in the subject. In some embodiments, the symptom is selected from the group consisting of depression, irritability, agitation, difficulty concentrating, tension, anger, stress, anxiety, and combinations thereof.

The presently disclosed methods can be employed for subjects with any neuropsychiatric disorder and/or for subjects with any one or more symptoms associated with a neuropsychiatric disorder. In some embodiments, the neuropsychiatric disorder is selected from the group consisting of schizophrenia, schizoaffective disorder, Alzheimer's disease or other neurodegenerative or cognitive disorder, Attention Deficit Disorder/Attention Deficit Hyperactivity Disorder, depression, bipolar disorder, post-traumatic stress disorder (PTSD), a pain disorder, tobacco dependence, alcohol abuse, alcohol dependence, drug dependence, drug abuse, traumatic brain injury, and combinations thereof.

The presently disclosed subject matter employs neuroactive steroid compositions comprising pregnenolone (PG), allopregnanolone (ALLO), dehydroepiandrosterone (DHEA), pharmaceutically acceptable salts thereof, derivatives thereof, or combinations thereof. In some embodiments, the neuroactive steroid combinations comprise at least two active agents selected from the group consisting of PG, ALLO, DHEA, pharmaceutically acceptable salts thereof, and derivatives thereof. In some embodiments, the derivative comprises a sulfated derivative. In some embodiments, the neuroactive steroid is utilized in a combination therapy to augment the efficacy of an existing pharmacologic agent such as, but not limited to an antidepressant, an anxiolytic, an antipsychotic, an anticonvulsant, or a mood stabilizer.

In some embodiments of the presently disclosed subject matter, the neuroactive steroid composition comprises an effective amount of pregnenolone (PG), allopregnanolone (ALLO), dehydroepiandrosterone (DHEA), pharmaceutically acceptable salts thereof, derivatives thereof, or combinations thereof. In some embodiments, the effective amount is sufficient to raise the level of pregnenolone (PG), allopregnanolone (ALLO), dehydroepiandrosterone (DHEA), derivatives thereof, or combinations thereof in a source selected from the group consisting of cerebrospinal fluid, serum, plasma, blood, saliva, skin, muscle, olfactory tissue, lacrimal fluid, synovial fluid, nail tissue, hair, feces, urine, in the subject by at least 1.5-fold within 8 weeks from a level in the source in the subject prior to the administering step. In some embodiments, the effective amount comprises a daily dose of at least 0.005 mg per day. In some embodiments, the effective dose comprises a dose ranging from about 0.005 mg to about 2000 mg of PG, ALLO, or DHEA, or an equivalent molar amount of the pharmaceutically acceptable salt thereof, the derivative thereof, or the combinations thereof. In some embodiments, the effective amount is sufficient to improve a cognitive function in the subject. In some embodiments, the neuroactive steroid composition comprises an effective amount of each of two or more of pregnenolone (PG), allopregnanolone (ALLO), dehydroepiandrosterone (DHEA), derivatives thereof, or combinations thereof.

In some embodiments of the presently disclosed subject matter, an effective amount is an amount sufficient to ameliorate one or more symptoms associated with a neuropsychiatric disorder in a subject. In some embodiments, the symptom is selected from the group consisting of a physical symptom, a psychological symptom, a negative symptom, or a cognitive symptom. In some embodiments, the physical symptom is selected from the group consisting of headache, nausea, diarrhea, tremor, insomnia or other sleep disturbance, restlessness, weight gain, appetite changes, and combinations thereof. In some embodiments, the psychological symptom is selected from the group consisting of depression, irritability, agitation, difficulty concentrating, tension, anger, stress, anxiety, and combinations thereof. In some embodiments, the negative symptom is selected from the group consisting of affective flattening, alogia, and avolition.

The presently disclosed neuroactive steroid compositions can also be administered as part of a combination therapy with one or more additional therapies that are appropriate for whatever condition the subject might have. As such, the presently disclosed methods further comprise in some embodiments administering to the subject at least one additional composition selected from the group consisting of an antidepressant, an anxiolytic, an antipsychotic, an anticonvulsant, and a mood stabilizer, wherein the at least one additional composition is administered to the subject before, after, and/or at the same time as the neuroactive steroid composition.

The presently disclosed neuroactive steroid compositions can be administered to a subject in any form and/or by any route of administration. In some embodiments, the formulation is a sustained release formulation, a controlled release formulation, or a formulation designed for both sustained and controlled release. In some embodiments, the sustained release formulation, the controlled release formulation, or the combination thereof is selected from the group consisting of an oral formulation, a peroral (PO) formulation, a buccal formulation, an enteral formulation, a pulmonary formulation, a rectal formulation, a vaginal formulation, a nasal formulation, a lingual formulation, a sublingual formulation, an intravenous formulation, an intraarterial formulation, an intracardial formulation, an intramuscular formulation, an intraperitoneal formulation, a transdermal formulation, an intracranial formulation, an intracutaneous formulation, a subcutaneous formulation, an aerosolized formulation, an ocular formulation, an implantable formulation, a depot injection formulation, and combinations thereof. In some embodiments, the route of administration is selected from the group consisting of oral, peroral, buccal, enteral, pulmonary, rectal, vaginal, nasal, lingual, sublingual, intravenous, intraarterial, intracardial, intramuscular, intraperitoneal, transdermal, intracranial, intracutaneous, subcutaneous, ocular, via an implant, and via a depot injection.

Accordingly, it is an object of the presently disclosed subject matter to provide methods for ameliorating and/or preventing the development of one or more symptoms associated with neuropsychiatric disorders in subjects.

An object of the presently disclosed subject matter having been stated hereinabove, and which is achieved in whole or in part by the presently disclosed subject matter, other objects will become evident as the description proceeds.

### BRIEF DESCRIPTION OF THE FIGURES

FIG. 1 is a bar graph showing that pregnenolone (PG) treatment of subjects with schizophrenia results in significant increases in pregnenolone sulfate (PGS). White box—baseline PGS level; black box—PGS level at 10 weeks.

FIG. 2 is a bar graph showing that subjects receiving PG demonstrated significantly greater reductions in negative symptoms as determined by SANS scores compared to subjects receiving placebo. White box—mean change in SANS Score for subjects receiving placebo; black box—mean change in SANS Score for subjects receiving PG.

FIGS. 3A-3D are a series of graphs showing that neuroactive steroid increases predict cognitive improvements in subjects with schizophrenia, and also that serum levels of PG and ALLO are highly correlated after PG administration. Each square corresponds to an individual subject.

FIGS. 3A and 3B are graphs showing that an increase in serum PG levels predict BACS (FIG. 3A) and MATRICS (FIG. 3B) improvement in subjects with schizophrenia.

FIG. 3C is a graph showing that that an increase in serum ALLO levels following PG administration predict BAGS improvement in subjects with schizophrenia.

FIG. 3D is a graph showing that serum PG and ALIO levels are highly correlated following PG administration in subjects with schizophrenia.

FIG. 4 is a bar graph showing that PG administration to subjects with schizophrenia or schizoaffective disorder did not negatively impact subjects' QTc intervals on EKG. White boxes—mean EKG QTc internal for subjects at baseline (visit 1); black boxes—mean EKG QTc internal for subjects at week 10 (visit 6).

FIG. 5 is a series of bar graphs showing median neuroactive steroid levels in parietal cortex in control subjects without a psychiatric diagnosis and in subjects with schizophrenia, bipolar disorder, and depression (non-psychotic), demonstrating that neuroactive steroids are altered in subjects with schizophrenia or bipolar disorder compared to control subjects. White boxes—control subjects; upwards hatching (left to right)—subjects with schizophrenia; black boxes—subjects with bipolar disorder; downwards hatching (left to right)—subjects with non-psychotic depression; # post-hoc Dunnett p=0.06; ** post-hoc Dunnett p<0.01; * post-hoc Dunnett p=0.04.

FIG. 6 is a series of bar graphs showing median neuroactive steroid levels in the posterior cingulate in control subjects without a psychiatric diagnosis and in subjects with schizophrenia, bipolar disorder, and depression (non-psychotic), further demonstrating that neuroactive steroids are altered in subjects with schizophrenia or bipolar disorder compared to control subjects. White boxes—control subjects; upwards hatching (left to right)—subjects with schizophrenia; black boxes—subjects with bipolar disorder; downwards hatching (left to right)—subjects with non-psychotic depression; ** post-hoc Dunnett p<0.01.

FIGS. 7A-7F are plots showing levels of neuroactive steroids in temporal cortex of subjects with Alzheimer's disease and correlations thereof with neuropathological disease stages (Braak and Braak).

FIGS. 7A and 7C are plots showing that the levels of PG and DHEA, respectively, are increased in temporal cortex of AD subjects relative to control subjects. Each diamond corresponds to an individual control subject and each triangle corresponds to an individual subject with Alzheimer's disease.

FIGS. 7B, 7D, and 7F are plots showing that PG levels (FIG. 7B) and DHEA levels (FIG. 7D) are positively correlated with neuropathological disease stage (Braak), whereas ALLO levels (FIG. 7F) are inversely correlated with neuropathological disease stage (Braak). Each diamond corresponds to an individual subject with Alzheimer's disease.

FIG. 7E is a plot showing that levels of ALLO are decreased in temporal cortex of AD subjects relative to control subjects. Each diamond corresponds to an individual control subject and each triangle corresponds to an individual subject with Alzheimer's disease.

FIG. 8 is a plot showing that APOE 4 allele status is associated with decreased ALLO levels in temporal cortex of subjects; individuals carrying an APOE 4 allele had significantly reduced levels of ALLO relative to individuals that did not carry an APOE 4 allele. Each circle corresponds to an individual subject that does not carry an APOE 4 allele, and each triangle corresponds to an individual subject that does carry an APOE 4 allele.

FIGS. 9A-9C are a series of graphs showing data derived from assaying PG levels in cerebrospinal fluid (CSF) of AD subjects.

FIG. 9A is a bar graph showing that PG in CSF tends to be elevated in AD subjects. White box—mean level of PG in the CSF for control subjects; black box—mean level of PG in the CSF for subjects having Alzheimer's disease.

FIG. 9B is a plot showing that PG levels in CSF are correlated with PG levels found in temporal cortex. Each square corresponds to an individual subject with Alzheimer's disease.

FIG. 9C is a plot showing that PG levels in CSF tend to be positively correlated with neuropathological disease stage (Braak). Each square corresponds to an individual subject with Alzheimer's disease.

FIGS. 10A-10C are a series of graphs showing data derived from assaying DHEA levels in cerebrospinal fluid (CSF) of AD subjects.

FIG. 10A is a bar graph showing that DHEA in CSF is elevated in AD subjects. White box—mean level of DHEA in the CSF for control subjects; black box—mean level of DHEA in the CSF for subjects having Alzheimer's disease.

FIG. 10B is a plot showing that DHEA levels in CSF are correlated with DHEA levels found in temporal cortex. Each square corresponds to an individual subject with Alzheimer's disease.

FIG. 10C is a plot showing that DHEA levels in CSF are positively correlated with neuropathological disease stage (Braak). Each square corresponds to an individual subject with Alzheimer's disease.

FIGS. 11A and 11B are plots showing that improvements in Self-rated Improvement Scale (SRS; FIG. 11A) and Davidson Trauma Scale (DTS; FIG. 11B) correlate with percent change in PG in PTSD subjects treated with sertraline. Each square corresponds to an individual subject.

FIG. 12 is a plot showing how statistical power varies with detectable differences (multiple of SD) of the outcome measure using the method of Schoenfeld disclosed in EXAMPLE 10.

FIG. 13 is a bar graph showing that mean ALLO levels in serum are reduced in male veterans reporting lower back pain. White box—mean level of serum ALLO for subjects reporting no or minimal lower back pain; black box—mean level of serum ALLO for subjects reporting moderate or severe lower back pain.

FIG. 14 is a bar graph showing that mean ALLO levels in serum are reduced in male veterans reporting chest pain. White box—mean level of serum ALLO for subjects reporting no or minimal chest pain; black box—mean level of serum ALLO for subjects reporting moderate or severe chest pain.

FIG. 15 is a bar graph showing that median allopregnanolone/progesterone (ALLO/PROG) ratios decrease as PTSD symptoms increase, suggesting a relative deficit in ALLO formation in subjects with PTSD. White box—DTS Score less than 20; hatched box—DTS Score 20-39; black box—DTS Score 40-59; cross hatched box—DTS Score greater than or equal to 60.

FIG. 16 is a bar graph showing that median ALLO/PROG ratios decrease as depression symptoms increase, suggesting a relative deficit in allopregnanolone formation in subjects with depression. White box—BDI-I1 Score less than 10; hatched box—BDI-I1 Score 10-19; black box—BDI-I1 Score greater than or equal to 20.

FIGS. 17A and 17B are bar graphs summarizing neuroactive steroid levels in rat frontal cortex following chronic lithium administration. White box—vehicle; hatched box—lithium treatment; black box—valproate treatment.

FIG. 17A shows that ALLO levels in rat frontal cortex following lithium administration are significantly increased compared to vehicle administration, and

FIG. 17B shows that PG levels in rat frontal cortex tend to be increased following lithium administration compared to vehicle administration.

FIG. 18 is a graph showing that ALLO levels were positively correlated with PG levels in rodent frontal cortex following chronic lithium administration. Each square corresponds to an individual animal.

FIG. 19 is a plot showing that serum ALLO levels were positively correlated with salivary cotinine levels in male smokers (Pearson r=0.57, p=0.006, n=22). Each diamond corresponds to an individual subject.

FIG. 20 is a plot showing that serum PG levels tend to be positively correlated with salivary cotinine levels in male smokers (Pearson r=0.40, p=0.066, n=22). Each diamond corresponds to an individual subject.

FIG. 21 is a bar graph showing that PG administration resulted in a five-fold increase in serum ALLO in subjects, suggesting that pregnenolone administration may constitute an effective precursor loading strategy for achieving elevations in allopregnanolone levels. White box—mean serum ALLO level at visit 2 (baseline); black box—mean serum ALLO level at visit 6 (week 10).

FIG. 22 is a schematic summary of the biosynthesis of neuroactive steroids and their precursors from cholesterol. Arrows show synthetic directions, and the names of the enzymes that catalyze the reactions are indicated adjacent to the arrows.

### BRIEF DESCRIPTION OF THE SEQUENCE LISTING

SEQ ID NOs: 1-4 are the nucleotide sequences of oligonucleotides that can be employed in the polymerase chain reaction (PCR) to genotype offspring from an intercross of F₁ animals generated by crossing BcI-2 KO mice (strain B6129S2-Bcl^{2tm1Sjk}/J; Stock Number 002265 of the Jackson Laboratory, Bar Harbor, Me.) to a wild type strain (B6129SF2/J; Number 101045 of the Jackson Laboratory). The Bcl-2 KO strain carries a neomycin resistance cassette a subsequence of which can be amplified using primers that have the sequences set forth in SEQ ID NOs: 1 and 2 to generate a 280 basepair fragment. A subsequence of the murine Bcl-2 gene can be amplified using primers that have the sequences set forth in SEQ ID NOs: 3 and 4 to generate a 215 basepair fragment.

### DETAILED DESCRIPTION

### I. General Considerations

Pregnenolone (PG) is a neurosteroid (i.e., a steroid synthesized de novo in the brain from cholesterol). Its sulfated derivative pregnenolone sulfate (PGS) is considered to be a “neuroactive steroid,” since it demonstrates effects at membrane-bound ligand-gated ion channel receptors such as N-methyl-D-aspartic acid (NMDA) receptors. PG and PGS enhance learning and memory in rodent models (Vallee et al., 1997; Vallee et al., 2000; Vallee et al., 2001; Akwa et al., 2001; Flood et al., 1992; Flood et al., 1995). These effects might be NMDA receptor-mediated. PGS also increases acetylcholine release in rodent hippocampus and cortex, and these actions represent another potential mechanism for its effects on learning and memory in rodent models. Other positive modulators of NMDA receptors (including glycine, serine, and D-cycloserine) might decrease negative symptoms in patients with schizophrenia (paucity of speech, avolition, anhedonia, affective flattening, etc.), and might also impact cognitive symptoms. PG is also elevated following certain antipsychotic agents and may contribute to their therapeutic efficacy (Marx et al., 2006a; Marx et al., 2006d)

Cognitive symptoms and negative symptoms in patients with schizophrenia are frequently severe, and strongly correlated with decreased functional outcome and quality of life. NMDA receptors are known to impact learning and memory. Cognitive deficits have been associated with poor treatment outcomes in subjects with certain neuropsychiatric disorders (NPDs) including, but not limited to schizophrenia. PG has been investigated for the treatment of rheumatoid arthritis and other disorders in humans, and shown to be safe, well-tolerated.

Subjects with NPDs (e.g., schizophrenia and schizoaffective disorder) frequently demonstrate significant cognitive deficits, and these deficits are more closely related to functional outcome than any other symptom domain (including “positive symptoms” such as auditory hallucinations and delusions). The newer antipsychotics (also referred to as “second-generation” or “atypical” antipsychotics) have only modest effects on cognitive outcomes. These newer agents do not appear to further impair cognitive functioning, however, a side effect frequently attributed to the older antipsychotics (also designated “conventional”, “first-generation”, or “typical” antipsychotics). The improved side effect profiles of the newer agents with regard to cognitive functioning represent progress, but effective agents to improve cognitive symptoms in NPDs such as schizophrenia and schizoaffective disorder still represent an urgent clinical need. Furthermore, a number of second generation antipsychotic agents have been associated with increased risk for weight gain, diabetes, and dyslipidemias. Thus, new agents with improved side effect profiles are needed.

Other evidence suggests that NMDA antagonists such as ketamine induce psychotic symptoms. Positive modulation of NMDA receptors might therefore improve symptoms of NPDs. Several studies demonstrating that agonists of the glycine modulatory site of the NMDA receptor may improve negative symptoms in some patients support this possibility (Goff et al., 1999; Heresco-Levy et al., 1999).

In recent years, the impact of cognitive deficits on patient functioning has been recognized (Green, 1996), and investigations into the amelioration of cognitive deficits found in NPDs have received increasing attention. It is possible that other positive modulators of NMDA receptors may have efficacy for neurocognitive symptoms in schizophrenia and other NPDs. Since the sulfated derivative of PG is a positive modulator of NMDA receptors and increases acetylcholine release in rodent hippocampus, PG, its . derivatives, and/or its metabolites might be helpful for cognitive symptoms in subjects with NPDs. In addition, second-generation antipsychotics such as clozapine and olanzapine elevate PG levels in rodent hippocampus to concentrations that are physiologically relevant. If antipsychotics also elevate PG in subjects with psychosis, it is possible that elevations in PG and other neuroactive steroids might contribute to antipsychotic therapeutic efficacy.

Therefore, disclosed herein are compositions and methods that in some embodiments target PG directly as an augmentation strategy in persistently symptomatic subjects with NPDs. An aspect of the present disclosure also determines PG levels at baseline and during PG augmentation, and characterizes PG metabolites such as dehydroepiandrosterone (DHEA) and the GABAergic neuroactive steroid allopregnanolone (ALLO). DHEA augmentation appears to be effective for negative symptoms and depressive symptoms in subjects with schizophrenia (Strous et al. , 2003), and might also impact cognitive symptoms (Strous et al., 2004). Since DHEA administration also elevates PG and ALLO levels in humans, other neuroactive steroids such as PG might be efficacious for cognitive symptoms and other symptom domains in subjects with NPDs.

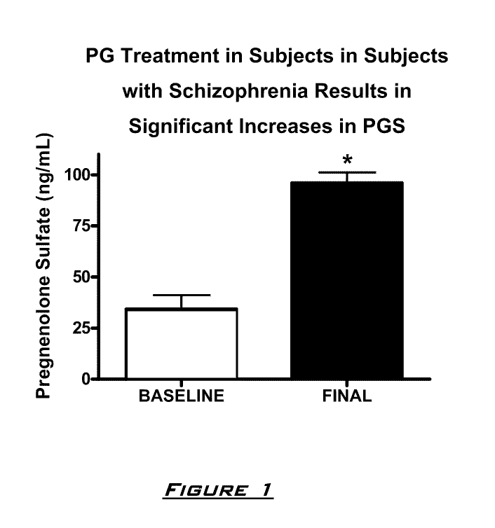

Orally administered PG appears to be well-absorbed and converted to its sulfated form (PGS; see FIG. 1). Studies in rodents have shown that both PG and PGS appear to be transported across the blood brain barrier. Thus, several studies have demonstrated that oral administration of PG is safe, well tolerated, and likely results in elevated brain levels of both PG and PGS.

Accordingly, in some embodiments the presently disclosed subject matter relates to PG augmentation for cognitive symptoms and other psychiatric symptoms (negative symptoms, depressive symptoms, positive symptoms) in persistently symptomatic subjects with NPDs such as, but not limited to schizophrenia and schizoaffective disorder.

The presently disclosed subject matter also relates to compositions comprising other neuroactive steroids and methods of using the same. One such neuroactive steroid is allopregnanolone (3α-hydroxy-5α-pregnan-20-one; ALLO). ALLO is synthesized de novo in the brain from cholesterol or from peripheral steroid precursors (Belelli & Lambert, 2005). A number of ALLO actions are attributed to the fact that it potentiates GABA_{A} receptor responses at nanomolar concentrations, doing so more potently than either benzodiazepines or barbiturates (Morrow et al., 1987). ALLO demonstrates anxiolytic (Wieland et al., 1991) and anticonvulsant effects (Kokate et al., 1996).

More recently, neuroprotective roles for ALLO have been demonstrated in a mouse model of Niemann-Pick type C disease (Griffin et al., 2004) and a rat model of traumatic brain injury (Djebaili et al., 2005). ALLO also protects against apoptosis via Bcl-2 protein in rat adrenal chromaffin and pheochromocytoma cells (Charalampopoulos et aL, 2004) and protects against N-methyl-D-aspartate (NMDA)-induced apoptosis in mouse P19-derived neurons (Xilouri & Papazafiri, 2006).

Nicotine dependence is an addiction with major health sequelae, exacting tremendous human and economic costs worldwide. Although significant progress has been achieved in reducing nicotine usage in recent years, effective pharmacological and behavioral smoking cessation interventions remain limited. Failure and dropout rates in smoking cessation clinical trials are high and long-term abstinence rates are suboptimal. New approaches are needed.

Disclosed herein are examinations of neuroactive steroid associations with nicotine dependence severity and negative affect rating measures, as well as nicotine and cotinine levels in male smokers. An aspect of the present disclosure pertains to neuroactive steroids as candidates for pharmacological targets for smoking cessation.

Neuroactive steroids can be synthesized in the brain (neurosteroids), adrenals, or gonads, and can rapidly alter neuronal excitability by acting at ligand-gated ion channel receptors, including NMDA and GABA_{A} receptors (Paul & Purdy, 1992; Rupprecht & Holsboer, 1999). For example, DHEA and PGS are positive modulators of excitatory NMDA receptors (Irwin et al., 1994; Wu et al., 1991; Compagnone & Mellon, 1998; Debonnel et al., 1996) and negative modulators of inhibitory GABA_{A} receptors (Majewska et al., 1988; Imamura & Prasad, 1998; Park-Chung et al., 1999). Conversely, neuroactive steroids such as ALLO are positive modulators of GABA_{A} receptors, potentiating GABA_{A} receptor response more potently than benzodiazepines or barbiturates (Majewska et al., 1986; Morrow et al., 1987, Morrow et al., 1990). ALLO increases with a number of acute stressors in rodent models (Purdy et al., 1991; Barbaccia et al., 1996, Barbaccia et al., 1998; Morrow et al., 1995; Vallee et al., 2000), and might represent a component of an endogenous regulatory mechanism that contributes to the modulation of hypothalamic-pituitary-adrenal (HPA) axis activity (Morrow et al., 1995). The HPA axis might be relevant to the pathophysiology of nicotine dependence, and rodent evidence suggests that nicotine administration dose-dependently increases ALLO and PG levels (Porcu et al., 2003). Although a number of studies have investigated nicotine and the HPA axis by targeting cortisol and corticosterone in clinical and preclinical models, respectively, data are currently more limited regarding other steroids that are also produced in the adrenal (as well as other sites), including ALLO, PG, and DHEAS. Disclosed herein for the first time are investigations and characterization of these latter steroids in the context of smoking cessation and the physical and/or psychological symptoms that can result from tobacco use and/or dependence.

Substantial evidence suggests that the hypothalamic-pituitary-adrenal (HPA) axis is indeed altered in smokers. Specifically, adrenocortical stimulating hormone (ACTH), cortisol, and corticosterone levels are increased following nicotine exposure in both humans and rodents (Rosecrans & Karin 1998; Gossain et al., 1986; Pomerleau et al., 2001; Matta et al., 1998; Caggiula et al., 1998; al'Absi et al., 2003; Tziomalos & Charsoulis 2004). Conversely, nicotine withdrawal states have been reported to result in cortisol decreases at from 3 days to as many as 6 weeks after smoking cessation (Puddey et al., 1984; Frederick et al., 1998; Meliska et al., 1995; Gilbert et al., 1999). Cortisol levels at various time points after smoking cessation, however, have been reported to be unchanged or increased, including after overnight (al'Absi et al., 2002), 24 hour (Hughes et al., 1988), 37 hour (Pickworth & Fant 1998), and 72 hour (Pickworth et al., 1996) abstinence periods.

It is possible that cortisol alterations following smoking cessation might have functional significance. For example, decreases in cortisol following smoking cessation at 2 weeks were correlated with degrees of withdrawal distress at the same time point (Frederick et al., 1998). Smokers who relapsed during the first week of cessation also had more pronounced decreases in morning cortisol levels and greater degrees of withdrawal symptoms during the first day of abstinence compared to subjects who maintained abstinence (al'Absi et al., 2004). Cortisol changes following smoking cessation might therefore have potential predictive value for withdrawal symptom severity and relapse likelihood.

In addition to cortisol, the neuroactive steroids dehydroepiandrosterone (DHEA), dehydroepiandrosterone sulfate (DHEAS), and androstenedione (ANDRO) also appear to be altered in subjects who smoke. A number of studies have demonstrated that DHEAS levels are higher in smokers compared to non-smokers (Bjornerem et al., 2004; Salvini et al., 1992; Barrett-Connor et al., 1986; Field et al., 1994; Hautanen et al., 1993; Khaw et aL, 1988; Baron et al., 1995; Laughlin & Barrett-Connor 2000; Feldman et al., 1998; Law et aL, 1997), and that DHEA (Field et al., 1994; Feldman et al., 1998) and ANDRO levels (Field et al., 1994; Hautanen et al., 1993; Khaw et al., 1988; Baron et al., 1995; Law et al., 1997) are also elevated in subjects who smoke. In addition, ACTH-stimulated ANDRO and DHEA levels appear to be higher in smokers (Hautanen et al., 1993). These steroids can be synthesized in the adrenals, and therefore data demonstrating higher DHEA, DHEAS, and ANDRO levels in smokers (and increased DHEA and ANDRO responses to ACTH) suggest a potential upregulation in HPA axis activity in subjects who smoke. Consistent with this possibility, DHEA levels appear to decrease after smoking cessation (Oncken et al., 2002), but it is unknown if these changes are associated with drawal symptom severity.

Nicotine administration also appears to alter steroids in addition to DHEAS, dose-dependently increasing the neuroactive steroids ALLO and PG in rodent cerebral cortex (Porcu et al., 2003). Therefore, DHEAS, ALLO, PG, ANDRO, free testosterone, and other steroid levels were examined in an aspect of the presently disclosed subject matter in 28 male smokers at baseline prior to randomization to specific smoking cessation treatment arms to determine potential associations with nicotine dependence severity and negative affect, and to begin to elucidate peripheral steroid profiles in this cohort.

Also potentially relevant to the area of nicotine dependence and neuroactive steroids are hypothesized interactions between smoking and negative affect, including depressive symptoms (Kassel et al., 2003; Paperwalla et al., 2004). Depressive symptoms are frequently reported during nicotine withdrawal and might constitute a risk factor for relapse (Glassman et al., 1990; And a et al., 1990; Pomerleau et al., 2001). Smokers with a history of depression might be at heightened risk for depressive symptom relapse following smoking cessation (Glassman et al., 2001; Covey et al., 1997). The precise mechanisms contributing to these findings are currently unknown, but it is possible that neuroactive steroids are relevant modulators of negative affect and depressive symptoms in subjects with tobacco dependence. Specifically, an expanding preclinical and clinical literature linking neuroactive steroids to a number of psychiatric disorders including depression (Uzunova et al., 1998; Wolkowitz et al., 2001; Schmidt et al., 2005) and schizophrenia (Strous et al., 2003; Marx et al., 2000, 2003; Barbaccia et al., 2001) supports this possibility. Furthermore, DHEA monotherapy in subjects with midlife depression appears to be an effective treatment intervention (Schmidt et al., 2005), and DHEA augmentation in subjects with schizophrenia decreases depressive and anxiety symptoms (Strous et al., 2003). In both studies, DHEA administration resulted in increased peripheral levels of its sulfated derivative DHEAS (Schmidt et al., 2005; Strous et al., 2003). Therefore, disclosed herein in an aspect of the presently disclosed subject matter are investigations to determine if DHEAS or other steroid levels are associated with the negative affect subscale of the Shiffman-Jarvik Withdrawal Questionnaire in male smokers.

### II. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the articles “a”, “an”, and “the” refer to “one or more” when used in this application, including in the claims. For example, the phrase “a symptom” refers to one or more symptoms. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the phrase “biological sample” refers to a sample isolated from a subject (e.g., a biopsy) or from a cell or tissue from a subject (e.g., RNA and/or DNA isolated therefrom). Biological samples can be of any biological tissue or fluid or cells from any organism as well as cells cultured in vitro, such as cell lines and tissue culture cells. Frequently the sample will be a “clinical sample” which is a sample derived from a subject (i.e., a subject undergoing a diagnostic procedure and/or a treatment). Typical clinical samples include, but are not limited to cerebrospinal fluid, serum, plasma, blood, saliva, skin, muscle, olfactory tissue, lacrimal fluid, synovial fluid, nail tissue, hair, feces, urine, a tissue or cell type, and combinations thereof, tissue or fine needle biopsy samples, and cells therefrom. Biological samples can also include sections of tissues, such as frozen sections or formalin fixed sections taken for histological purposes.

As used herein, the term “subject” refers to any organism for which diagnosis, treatment, and/or prophylaxis would be desirable. Thus, the term “subject” is desirably a human subject, although it is to be understood that the principles of the presently disclosed subject matter indicate that the presently disclosed subject matter is effective with respect to other species, including mammals, which are intended to be included in the term “subject”. Moreover, a mammal is understood to include any mammalian species for which diagnosis, treatment, and/or prophylaxis is desirable, particularly agricultural and domestic mammalian species. The methods of the presently disclosed subject matter are particularly useful in the diagnosis, treatment, and/or prophylaxis of warm-blooded vertebrates, e.g., mammals and birds.

More particularly, the presently disclosed subject matter can be used for diagnosis, treatment, and/or prophylaxis of a mammal such as a human. Also provided are methods for diagnosis, treatment, and/or prophylaxis of, and/or ameliorating a symptom associated with a neuropsychiatric disorder in, mammals of importance due to being endangered (such as Siberian tigers), of economic importance (animals raised on farms for consumption by humans) and/or social importance (animals kept as pets or in zoos) to humans.

As used herein, the term “prophylaxis” and grammatical variants thereof are intended to refer to the prevention, inhibition, and/or lessening of the development of a symptom associated with neuropsychiatric disorder (NPD) in a subject whether that symptom is already present or not. As such, “prophylaxis” is not intended to refer only to modulating the development of a symptom in a subject in which the symptom is completely absent but is also intended to refer to ameliorating the symptom in a subject in which it exists as well as preventing, inhibiting, and/or lessening any worsening of the symptom in the subject that might occur for any reason. Thus, the term “prophylaxis” is intended to overlap with yet be broader than the term “ameliorate”.

### III. Treatment and/or Prophylaxis Methods

Serum pregnenolone (PG) increases following PG administration. Subjects with greatest increases in serum PG have the greatest improvements in cognitive symptoms by two art recognized cognitive assessment batteries. Clozapine (current treatment) increases PG in rat hippocampus, cerebral cortex, and serum. PG is significantly reduced in parietal cortex in subjects with schizophrenia that died by suicide versus other causes of death. ALLO is elevated following clozapine administration in rodent models. PG administration increases serum ALLO in schizophrenic subjects over 5-fold.

ALLO is significantly reduced in postmortem prefrontal cortex and temporal cortex brain tissue in AD subjects, and is inversely correlated with neuropathological disease state. DHEA levels are elevated in temporal and prefrontal cortex of AD subjects. PG levels are increased in temporal cortex. DHEA and PG are elevated in cerebrospinal fluid of AD subjects, and are strongly correlated with DHEA and PG levels in temporal cortex

The presently disclosed subject matter thus provides methods for ameliorating a symptom of a neuropsychiatric disorder in a subject. Also provided are methods for ameliorating at least one physical symptom and/or at least one psychological symptom resulting from tobacco cessation in a subject; for ameliorating a symptom of Alzheimer's disease or other cognitive disorder in a subject; for ameliorating a symptom of schizophrenia, schizoaffective disorder, or other psychotic disorder in a subject; for ameliorating a symptom of a depressive disorder or other mood disorder in a subject; for ameliorating a symptom of bipolar disorder in a subject; for ameliorating a symptom of post-traumatic stress disorder or other anxiety disorder in a subject; for ameliorating a symptom of a pain disorder including, but not limited to a chronic pain disorder in a subject; for ameliorating a symptom of a sleep disorder in a subject; for ameliorating a symptom of a neurodegenerative disorder in a subject; for ameliorating a symptom of traumatic brain injury and/or concussion in a subject; and for ameliorating a symptom of an alcohol use disorder or other substance use disorder in a subject.

In some embodiments, the presently disclosed methods relate to ameliorating at least one physical symptom or at least one psychological symptom resulting from tobacco cessation in a subject. The methods of the presently disclosed subject matter can be employed before the subject has in fact ceased smoking, as well as during the period of time after which smoking has ceased but during which physical and/or psychological symptoms of nicotine use and/or dependence continue to be experienced by the subject. As such, it is understood that the compositions and methods of the presently disclosed subject matter can be employed as aids to smoking cessation as well as aid to preventing relapse into smoking. Therefore, the phrase “smoking cessation” is to be interpreted broadly to refer to any time before, during, and/or after such time as the subject has in fact ceased smoking.

As used herein, the term “neuropsychiatric disorder” is intended to refer broadly to any disorder of emotional, personality, and/or mental function that is of neurological origin, psychiatric origin, psychological origin, or mixed origin that negatively impacts the emotional and/or cognitive functioning of a subject. Representative neuropsychiatric disorders include those listed in the Diagnostic and Statistical Manual of Mental Disorders (DSM; including DSM-IV and DSM-IV-TR). More particularly, the term includes, but is not limited to such exemplary conditions as substance use disorders (e.g., use, abuse, and/or dependence on cocaine, opioid, cannabis, amphetamine, alcohol, caffeine, tobacco/nicotine, hallucinogens); anxiety disorders (e.g., post-traumatic stress disorder, obsessive compulsive disorder, panic disorder, agoraphobia, social phobia, acute stress disorder, generalized anxiety disorder, substance-induced anxiety disorder); mood disorders (e.g., both depressive and manic disorders including but not limited to major depressive disorder, major depressive disorder with psychotic features, major depressive disorder with postpartum onset, dysthymic disorder, bipolar I disorder, bipolar II disorder, cyclothymic disorder, substance-induced mood disorder); psychotic disorders (e.g., schizophrenia, schizoaffective disorder, delusional disorder, brief psychotic disorder, shared psychotic disorder, psychotic disorder due to a medical condition, substance-induced psychotic disorder, psychotic disorder not otherwise specified); cognitive disorders (e.g., mild cognitive impairment, Alzheimer's disease, vascular dementia, dementia due to other medical conditions, dementia due to multiple etiologies, substance-induced persisting amnestic disorder, amnestic disorder not otherwise specified, delirium). In some embodiments, the neuropsychiatric disorder is selected from the group consisting of schizophrenia, schizoaffective disorder, Alzheimer's disease, Attention Deficit Disorder/Attention Deficit Hyperactivity Disorder, depression, bipolar disorder, post-traumatic stress disorder (PTSD), a pain disorder, tobacco dependence, alcohol abuse, alcohol dependence, drug dependence, drug abuse, neurodegenerative disorders, sleep disorders, traumatic brain injury and/or concussion, and combinations thereof.

As used herein, the phrase “ameliorating a symptom” and grammatical variants thereof refers to providing an improvement in a symptom of a neuropsychiatric disorder in a subject. The improvement can be by any measure whatsoever, including a subjective assessment by the subject him or herself. Thus, the methods provided herein can ameliorate a symptom associated with a neuropsychiatric disorder to a degree that is measurable using some clinical criterion, which is measurable by employing one or more emotional and/or cognitive tests, or combinations thereof. The nature and extent of the amelioration of the symptom associated with a neuropsychiatric disorder does not limit the presently disclosed subject matter. One of ordinary skill in the art is familiar with methods to measure amelioration of symptoms.

As used herein, the phrase “symptom associated with a neuropsychiatric disorder” refers to any symptom that is typically observed in a subject that has a neuropsychiatric disorder, whether or not that subject does in fact have a neuropsychiatric disorder. Representative such symptoms include those set forth in the DSM (e.g., DSM-IV and/or DSM-IV-TR), each of which is expressly incorporated herein by reference in its entirety. A symptom associated with a neuropsychiatric disorder can be a physical symptom, a psychological symptom, a negative symptom, a cognitive symptom, or combinations thereof. Representative physical symptoms include, but are not limited to dizziness, lightheadedness, chest/abdominal pain, nausea, increased heart rate, headache, diarrhea, tremor, insomnia or other sleep disturbance, restlessness, weight gain, and appetite changes. Representative psychological symptoms include, but are not limited to depression, irritability, agitation, difficulty concentrating, tension, anger, stress, and anxiety. Representative negative symptoms include, but are not limited to affective flattening, alogia, and avolition. Representative cognitive symptoms include, but are not limited to forgetfulness, concentration difficulty, confusion, disorientation, dementia, delirium, learning disability, mental retardation, delusions, paranoia, hallucinations, disorganization, and indecision.

The presently disclosed subject matter also provides methods for improving cognitive functioning in a subject. As used herein, the phrase “improving cognitive functioning” refers to improving the cognitive functioning of the subject under any subjective or objective measure. One of ordinary skill in the art is aware of proper conditions under which to assess cognitive functioning, which can include various tests that are commonly employed. Representative, non-limiting tests include, but are not limited to neuropsychological tests such as the Continuous Performance Test (CPT), Wisconsin Card Sorting Test, Trailmaking A+B, the Mini Mental State Exam (MMSE), List Learning (Verbal Memory), Digit Sequencing Task (Working Memory), Token Motor Task (Motor Speed), Category Instances (Semantic Fluency), Controlled Oral Word Association Test (Letter Fluency), Tower of London Test (Executive Function), Symbol Coding (Attention and Motor Speed), Affective Interference Test-Delayed Recognition Task, Stroop Test, the Brief Assessment of Cognition in Schizophrenia (BACS; includes a number of the tests above), tests included in the Measurement and Treatment Research to Improve Cognition in Schizophrenia battery (MATRICS), and the Alzheimer's Disease Assessment Scale-Cognitive Subscale (ADAS-cog).

The presently disclosed subject matter also provides methods for delaying or preventing the onset of, and/or decreasing the severity of, a symptom associated with a neuropsychiatric disorder in a subject. In some embodiments, a neuroactive steroid composition is thus administered as a therapeutic to maintain a current state of well-being of a subject with a neuropsychiatric disorder. Thus, in some embodiments a neuroactive steroid composition of the presently disclosed subject matter is administered to a subject as a maintenance therapy to prevent the worsening of symptoms that subjects with a given neuropsychiatric disorder typically have and/or are at risk of developing.

In some embodiments, the subject does not have a neuropsychiatric disorder but is at risk for developing one or more symptoms that are associated with a neuropsychiatric disorder, whether or not the subject develops a recognized neuropsychiatric disorder. The development of such symptoms can accompany the subject entering into a situation where stress, anxiety, depression, and/or other hallmarks of neuropsychiatric disorders can be elicited in an otherwise healthy subject. These situations can include normal day-to-day activities that would be expected to cause stress, anxiety, and/or depression, but can also include extraordinary activities including, but not limited to entry into combat. The development of such symptoms can also occur as a result of other biochemical and biological alterations in the subject that are not caused by a neuropsychiatric disorder including, but not limited to the onset of menopause.

### IV. Methods for Predicting a Predisposition to Suicide, Suicidal Ideation, Suicidal Behavior, or Combinations Thereof

The presently disclosed subject matter also provides methods for predicting a predisposition to suicide, suicidal ideation, suicidal behavior, or a combination thereof in a subject. In some embodiments, the methods comprise (a) determining a level of one or more neuroactive steroids in a sample isolated from the subject; and (b) comparing the level determined in step (a) to a standard, wherein the level of the one or more neuroactive steroids in the sample compared to the level in the standard is indicative of a predisposition to suicide, suicidal ideation, suicidal behavior, or combinations thereof.

In some embodiments, the sample is selected from the group consisting of cerebrospinal fluid, serum, plasma, blood, saliva, skin, muscle, olfactory tissue, lacrimal fluid, synovial fluid, nail tissue, hair, feces, urine, a tissue or cell type, and combinations thereof.

In some embodiments, the standard comprises a sample from a subject that does not have a predisposition to suicide, suicidal ideation, suicidal behavior, or combinations thereof. In some embodiments, the standard is a recorded value recognized by those of ordinary skill in the art to be associated with a normal state; i.e., a state in which a reference subject does not have a predisposition to suicide, suicidal ideation, suicidal behavior, or combinations thereof.

### V. Neuroactive Steroid Compositions

The presently disclosed subject matter employs neuroactive steroid compositions comprising PG, ALLO, DHEA, pharmaceutically acceptable salts thereof, derivatives thereof, or combinations thereof. In some embodiments, the neuroactive steroid combinations comprise at least two active agents selected from the group consisting of PG, ALLO, DHEA, pharmaceutically acceptable salts thereof, and derivatives thereof. In some embodiments, the derivative comprises a sulfated derivative.

In some embodiments of the presently disclosed subject matter, the neuroactive steroid composition comprises an effective amount of PG, ALLO, DHEA, pharmaceutically acceptable salts thereof, derivatives thereof, or combinations thereof. In some embodiments, the effective amount is sufficient to raise the level of PG, ALLO, DHEA, derivatives thereof, or combinations thereof in a source selected from the group consisting of cerebrospinal fluid, serum, plasma, blood, saliva, skin, muscle, olfactory tissue, lacrimal fluid, synovial fluid, nail tissue, hair, feces, urine, in the subject by at least 1.5-fold within 8 weeks from a level in the source in the subject prior to the administering step. In some embodiments, the effective amount comprises a daily dose of at least 0.005 mg per day. In some embodiments, the effective dose comprises a dose ranging from about 0.005 mg to about 2000 mg of PG, ALLO, or DHEA, or an equivalent molar amount of the pharmaceutically acceptable salt thereof, the derivative thereof, or the combinations thereof. In some embodiments, the effective amount is sufficient to improve a cognitive function in the subject. In some embodiments, the neuroactive steroid composition comprises an effective amount of each of two or more of PG, ALLO, DHEA, derivatives thereof, or combinations thereof.

V.A. Formulations

A neuroactive steroid composition as described herein preferably comprises a composition that includes a pharmaceutically acceptable carrier. Suitable formulations include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents.

The compositions used in the methods can take such forms as suspensions, solutions, or emulsions in oily or aqueous vehicles, and can contain formulatory agents such as suspending, stabilizing, and/or dispersing agents. The compositions used in the methods can take such forms as inhalational formulations as well as oral and IV, including but not limited to fine powder formulations and droplet-generating formulations. Alternatively or in addition, the active ingredient can be in powder form for constitution with a suitable vehicle (e.g., sterile pyrogen-free water) before use.

The formulations can be presented in unit-dose or multi-dose containers, for example sealed ampules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulfate). The tablets can be coated by methods known in the art. For example, a neuroactive steroid can be formulated in combination with hydrochlorothiazide, and as a pH stabilized core having an enteric or delayed-release coating which protects the neuroactive steroid until it reaches the colon.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring, and sweetening agents as appropriate. Preparations for oral administration can be suitably formulated to give controlled release of the active compound. For buccal administration the compositions can take the form of tablets or lozenges formulated in conventional manner.

The compounds can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The compounds can also be formulated in rectal compositions (e.g., suppositories or retention enemas containing conventional suppository bases such as cocoa butter or other glycerides), creams or lotions, or transdermal patches.

In some embodiments, the presently disclosed subject matter employs a neuroactive steroid composition that is pharmaceutically acceptable for use in humans. One of ordinary skill in the art understands the nature of those components that can be present in a neuroactive steroid composition that is pharmaceutically acceptable for use in humans and also what components should be excluded from a neuroactive steroid composition that is pharmaceutically acceptable for use in humans.

V.B. Doses

The term “effective amount” is used herein to refer to an amount of the therapeutic composition (e.g., a composition comprising a neuroactive steroid, a pharmaceutically acceptable salt thereof, a derivative thereof, or a combination thereof) sufficient to produce a measurable biological response (e.g., an amelioration of a symptom). Actual dosage levels of active ingredients in a neuroactive steroid composition of the presently disclosed subject matter can be varied so as to administer an amount of the active compound(s) that is effective to achieve the desired response for a particular subject and/or application. The selected dosage level can depend upon a variety of factors including the activity of the neuroactive steroid composition, formulation, route of administration, combination with other drugs or treatments, severity of the condition being treated, and physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are known to those of ordinary skill in the art.

For administration of a neuroactive steroid composition as disclosed herein, conventional methods of extrapolating human dosage based on doses administered to a murine animal model can be carried out using techniques known to one of ordinary skill in the art. Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al., 1966. Briefly, to express a mg/kg dose in any given species as the equivalent mg/m² dose, multiply the dose by the appropriate km factor. In an adult human, 100 mg/kg is equivalent to 100 mg/kg 37 kg/m²=3700 mg/m².

In some embodiments of the presently disclosed subject matter, the neuroactive steroid composition comprises an effective amount of PG, ALLO, DHEA, pharmaceutically acceptable salts thereof, derivatives thereof, or combinations thereof. In some embodiments, the effective amount is sufficient to raise the level of PG, ALLO, DHEA, derivatives thereof, or combinations thereof in a source selected from the group consisting of cerebrospinal fluid, serum, plasma, blood, saliva, skin, muscle, olfactory tissue, lacrimal fluid, synovial fluid, nail tissue, hair, feces, urine, in the subject by at least 1.5-fold within 8 weeks from a level in the source in the subject prior to the administering step. In some embodiments, the effective amount comprises a daily dose of at least 0.005 mg per day. In some embodiments, the effective dose comprises a dose ranging from about 0.005 mg to about 2000 mg of PG, ALLO, or DHEA, or an equivalent molar amount of the pharmaceutically acceptable salt thereof, the derivative thereof, or the combinations thereof. In some embodiments, the effective amount is sufficient to improve a cognitive function in the subject. In some embodiments, the neuroactive steroid composition comprises an effective amount of each of two or more of PG, ALLO, DHEA, derivatives thereof, or combinations thereof.

For additional guidance regarding formulation and dose, see U.S. Pat. Nos. 5,326,902; 5,234,933; PCT International Publication No. WO 93/25521; Berkow et al., 1997; Goodman et al., 1996; Ebadi, 1998; Katzung, 2001; Remington et al., 1975; Speight et al., 1997; Duch et al., 1998.

V.C. Routes of Administration

The presently disclosed neuroactive steroid compositions can be administered to a subject in any form and/or by any route of administration. In some embodiments, the formulation is a sustained release formulation, a controlled release formulation, or a formulation designed for both sustained and controlled release. As used herein, the term “sustained release” refers to release of an active agent such that an approximately constant amount of an active agent becomes available to the subject over time. The phrase “controlled release” is broader, referring to release of an active agent over time that might or might not be at a constant level. Particularly, “controlled release” encompasses situations and formulations where the active ingredient is not necessarily released at a constant rate, but can include increasing release over time, decreasing release over time, and/or constant release with one or more periods of increased release, decreased release, or combinations thereof. Thus, while “sustained release” is a form of “controlled release”, the latter also includes delivery modalities that employ changes in the amount of an active agent (e.g., a neuroactive steroid composition) that are delivered at different times.

In some embodiments, the sustained release formulation, the controlled release formulation, or the combination thereof is selected from the group consisting of an oral formulation, a peroral formulation, a buccal formulation, an enteral formulation, a pulmonary formulation, a rectal formulation, a vaginal formulation, a nasal formulation, a lingual formulation, a sublingual formulation, an intravenous formulation, an intraarterial formulation, an intracardial formulation, an intramuscular formulation, an intraperitoneal formulation, a transdermal formulation, an intracranial formulation, an intracutaneous formulation, a subcutaneous formulation, an aerosolized formulation, an ocular formulation, an implantable formulation, a depot injection formulation, a transdermal formulation and combinations thereof. In some embodiments, the route of administration is selected from the group consisting of oral, peroral, buccal, enteral, pulmonary, rectal, vaginal, nasal, lingual, sublingual, intravenous, intraarterial, intracardial, intramuscular, intraperitoneal, transdermal, intracranial, intracutaneous, subcutaneous, ocular, via an implant, and via a depot injection. Where applicable, continuous infusion can enhance drug accumulation at a target site (see, e.g., U.S. Pat. No. 6,180,082). See also U.S. Pat. Nos. 3,598,122; 5,016,652; 5,935,975; 6,106,856; 6,162,459; 6,495,605; and 6,582,724; and U.S. Patent Application Publication No. 20060188558 for transdermal formulations and methods of delivery of compositions.

The particular mode of drug administration used in accordance with the methods of the presently disclosed subject matter depends on various factors, including but not limited to the vector and/or drug carrier employed, the severity of the condition to be treated, and mechanisms for metabolism or removal of the drug following administration.

### EXAMPLES

The following Examples provide illustrative embodiments. Certain aspects of the following Examples are disclosed in terms of techniques and procedures found or contemplated by the present inventors to work well in the practice of the embodiments. In light of the present disclosure and the general level of skill in the art, those of skill will appreciate that the following Examples are intended to be exemplary only and that numerous changes, modifications, and alterations can be employed without departing from the scope of the presently claimed subject matter.

### Example 1

### PG Administration to Subjects with Schizophrenia or Schizoaffective Disorder

The effects of PG on neurocognitive and negative symptoms in subjects with schizophrenia or schizoaffective disorder receiving stable doses of second generation antipsychotics were investigated in a randomized placebo-controlled double-blind trial. Following a two-week placebo lead-in phase of all subjects, subjects were randomized to PG or placebo for eight weeks. Dosages for the PG subjects were 50 mg bis in diem (BID) for 2 weeks, 150 mg BID for 2 weeks, and then 250 mg BID for 4 weeks.

After completing the 10 week dosing schedule, subjects were tested with the Brief Assessment of Cognition in Schizophrenia (BACS) and Measurement and Treatment Research to Improve Cognition in Schizophrenia (MATRICS) cognitive batteries. Subjects were also assessed by the Scale for the Assessment of Negative Symptoms (SANS) assessments at baseline (following completion of placebo lead-in), at 4 weeks, and 8 weeks. PG levels were determined by gas chromatography/mass spectrometry as described hereinbelow. PGS levels were also assayed, and were found to be significantly increased in subjects with schizophrenia that were administered PG (see FIG. 1).

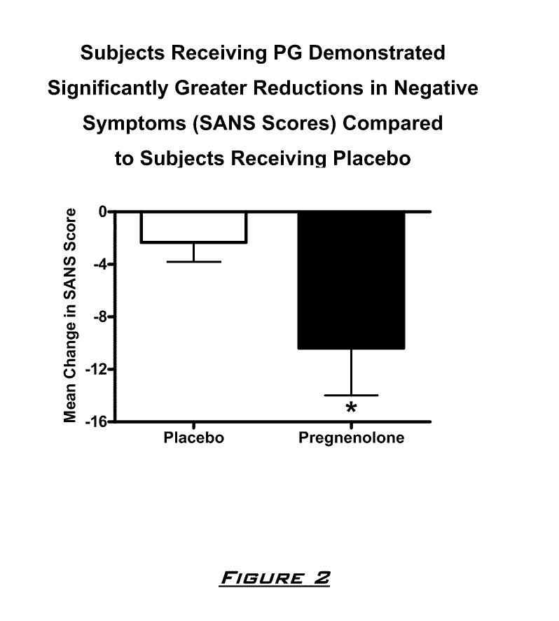

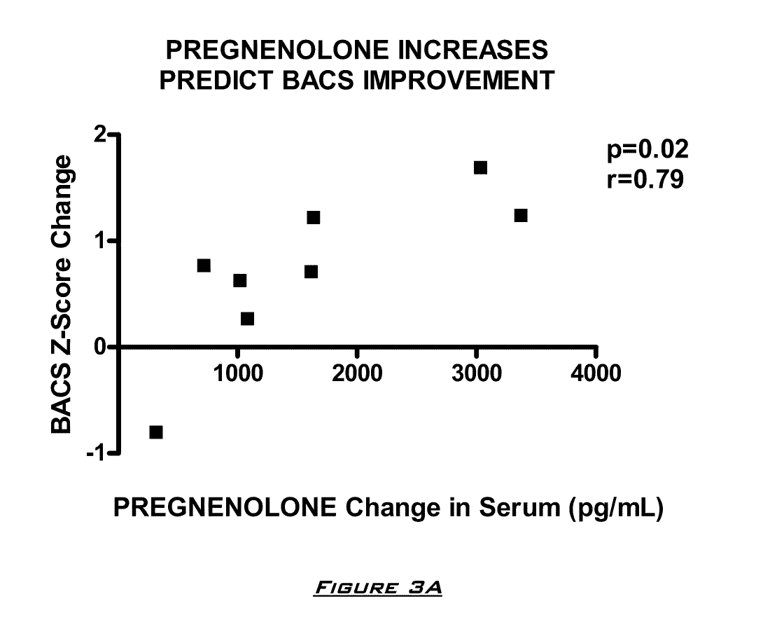

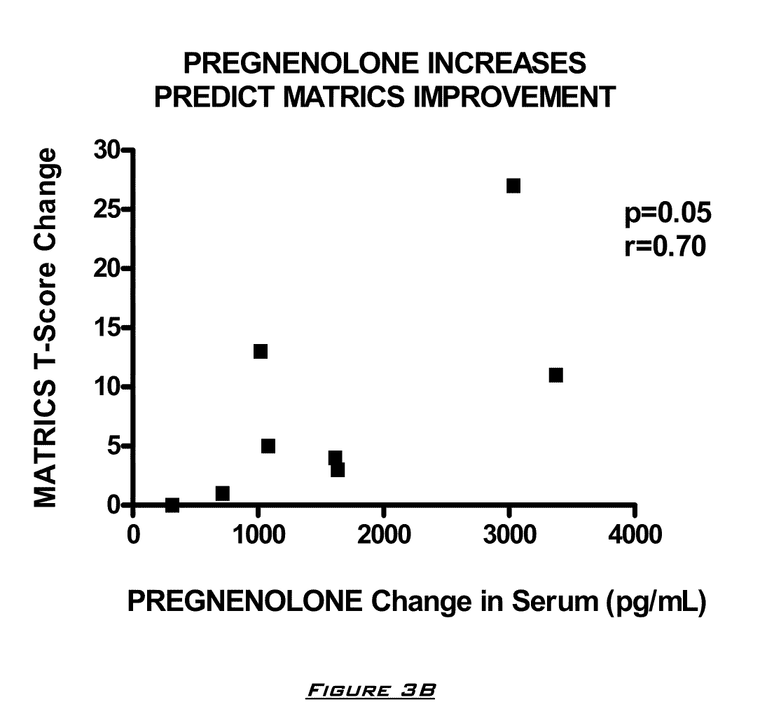

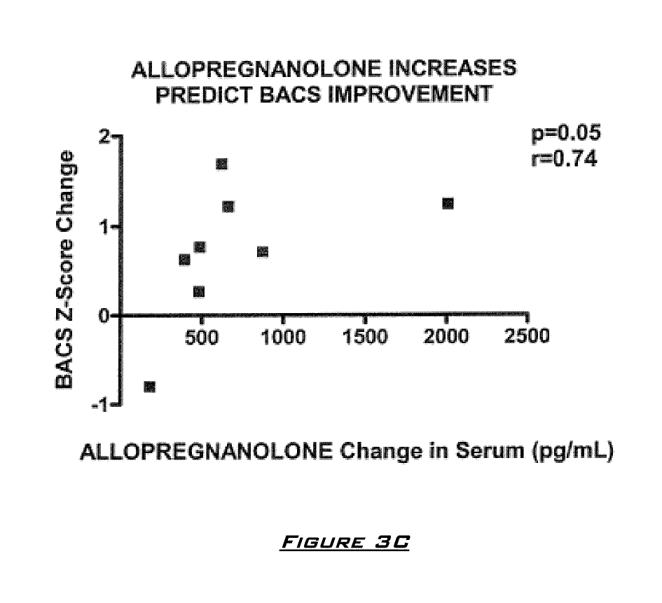

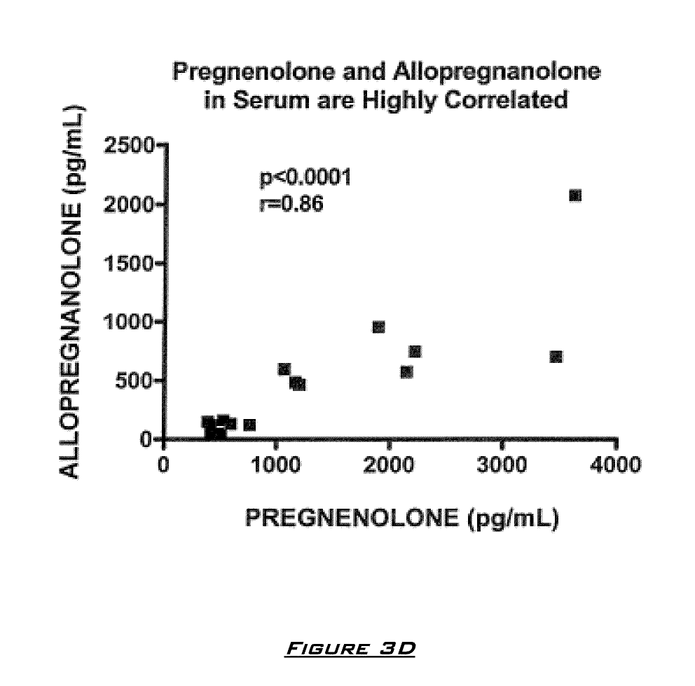

Of 21 subjects randomized, 18 completed at least four weeks of treatment (n=9 per group). Subjects receiving PG demonstrated significantly greater improvements in SANS scores (mean change 10.38) compared to subjects receiving placebo (mean change 2.33; p=0.05; see FIG. 2). Increases in serum PG levels were positively correlated with cognitive improvement in the PG group, predicting both BACS (r=0.79, n=9, p=0.02; see FIG. 3A) and MATRICS (r=0.70, n=9, p=0.05; see FIG. 3B) composite scores at eight weeks. The mean BACS z-score increase for the PG group was 0.60 (vs. 0.22 for the placebo group). BACS and MATRICS assessments were correlated (r=0.74, p<0.0001).

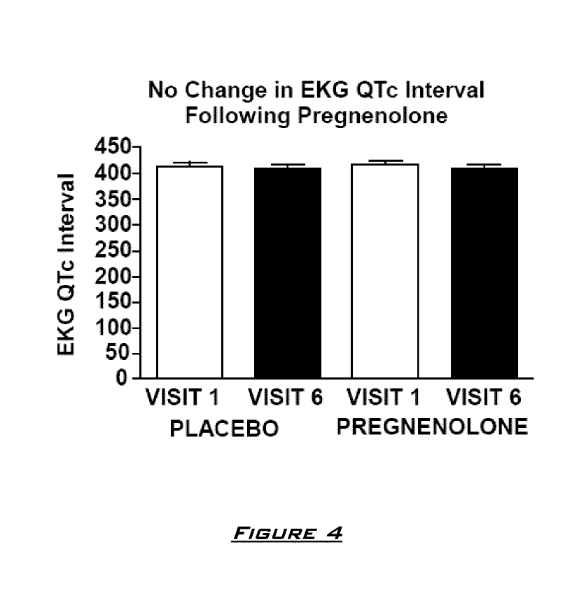

PG was well-tolerated (see Table 1) and did not negatively impact subjects' QTc intervals (see FIG. 4). Additionally, PG administration did not result in a significant change in AIMS, Barnes Akathisia Scale, Simpson-Angus Scale, blood pressure, pulse, or weight. Glucose levels increased slightly (average baseline 117.9 mg/dl vs. average post-treatment 119.0 mg/dl), and cholesterol decreased slightly (average baseline 185.8 mg/dl vs. average post-treatment 164.6 mg/dl).

TABLE 1
Side Effects in Subjects Randomized to PG vs. Placebo

| | PLACEBO | PG |
|---|---|---|
| | (n = 9) | (n = 9) |
| SYMPTOM | N (%) | N (%) |
| Disorientation (to date, address, | 2 (22%)^{a} | 2 (22%)^{a} |
| mayor, or MD name) | | |
| Decreased Interest in Sex | 2 (22%)^{a} | 1 (11%)^{a} |
| Impaired Sexual Performance | 2 (22%)^{a} | 0 |
| Hypertension | 1 (11%)^{b} | 1 (11%)^{b} |
| Excitation/Agitation | 1 (11%)^{a} | 0 |
| Dry mouth | 1 (11%)^{a} | 1 (11%)^{a} |
| Malaise | 1 (11%)^{a} | 0 |
| Blurred Vision | 1 (11%)^{a} | 0 |
| Restlessness | 0 | 2 (22%)^{a} |
| Muscle Pain/Stiffness | 0 | 1 (11%)^{a} |
| Cold Extremities | 0 | 1 (11%)^{a} |
| Tremor | 0 | 0 |
| Headache | 0 | 0 |
| Insomnia | 0 | 0 |
| Drowsiness | 0 | 0 |
| Rigidity | 0 | 0 |
| Akathisia | 0 | 0 |
| Diarrhea | 0 | 0 |
| Nasal Congestion | 0 | 0 |
| Sweating | 0 | 0 |
| Joint Pain/Stiffness | 0 | 0 |
| Peripheral Edema | 0 | 0 |
| All Other Side Effects | 0 | 0 |

Values are change from baseline pre-randomization (Hillside AE Form).
Key to superscripts:
^{a}mild in all subjects;
^{b}mild (<145/90).

Serum ALLO levels were also determined. Increases in serum ALLO levels were positively correlated with cognitive improvement in the PG group, predicting BACS (r=0.74, n=8, p=0.05; see FIG. 3C) composite scores at eight weeks.

And finally, PG and ALLO levels in serum were found to be highly correlated (see FIG. 3D) in these subjects, suggesting that PG is metabolized to ALLO in subjects that have received exogenous PG.

### Materials and Methods for Example 2

Postmortem Tissue: Postmortem tissue was generously donated by the Stanley Foundation Neuropathology Consortium. Frozen parietal cortex and posterior cingulate tissue were analyzed for neuroactive steroids by negative ion chemical ionization gas chromatography/mass spectrometry (GC/MS) preceded by high performance liquid chromatography (HPLC). Levels of the neuroactive steroids PG, DHEA, and allopregnanolone (ALLO) were determined in parietal cortex and posterior cingulate from 59-60 subjects (15 with schizophrenia, 15 with bipolar disorder, 14-15 with depression, and 15 non-psychiatric control subjects). Posterior cingulate tissue was unavailable for one subject with depression, and therefore 14 specimens were analyzed in this group. Subjects were group matched for age, sex, ethnicity, brain pH, and postmortem interval. Statistical analyses were performed by Mann-Whitney U statistic in subjects with schizophrenia who died by suicide compared to subjects with schizophrenia who died of other causes.

Since prior reports demonstrated similar neuroactive steroid alterations in subjects with schizophrenia and bipolar disorder (Marx et al., 2006b), statistical analyses were also performed for this combined group.

Neuroactive Steroid (NS) Analyses: Gas Chromatoqraphy/Mass Spectrometry (GC/MS), preceded by high performance liquid chromatography (HPLC): Neuroactive steroid analyses in frozen parietal cortex and posterior cingulate were performed as previously described (Dong et al., 2001; Uzunova et al., 1998) with minor modifications as set forth in Marx et al., 2006b. All glassware was silanized prior to the experiment.

Brain tissue and standards were homogenized in distilled water containing tritiated NS (New England Nuclear/PerkinElmer Life And Analytical Sciences, Inc., Waltham, Mass.) to detect the HPLC fractions of interest. In addition, deuterated PG and deuterated ALLO were added as the internal standards. NS were extracted using ethyl acetate and concentrated to dryness prior to HPLC. Each steroid was collected based upon the retention time of its radioactive analogue. The HPLC fractions containing PG, DHEA, and ALLO were evaporated to dryness and derivatized utilizing heptafluorobutyric acid anhydride (HFBA) in ethyl acetate. Derivatized standards and samples were injected onto an Agilent 5973 GC/MS (Agilent Technologies, Inc., Santa Clara, Calif.) in the negative ion chemical ionization (NICI) mode utilizing methane as the reaction gas and helium as the carrier gas.

In addition to the retention time of each steroid, the structural identification of each NS assayed was provided by its unique mass fragmentation pattern. Mass spectrometer single ion monitoring (SIM) mode was used to focus on the most abundant ion fragment for each steroid derivative. For NS quantification, the standard curve for the steroid of interest was prepared by combining varying known quantities of the steroid (Steraloids, Inc., Newport, Rhode Island) ranging from 2 to 3000 pg with a constant amount of the respective deuterated internal standard. Only peaks with a signal to noise ratio greater than or equal to 5:1 were integrated. The limit of NS detection with this method was 10 pg for PG and 2 pg for ALLO and DHEA.

### Example 2

### PG Alterations in Parietal Cortex are Associated with Death by Suicide in Subjects with Schizophrenia and Bipolar Disorder

Schizophrenia is associated with a very high lifetime risk of suicide and suicidal behaviors (Palmer et al., 2005). It has been reported that PG levels are elevated similarly in subjects with schizophrenia and bipolar disorder in both parietal cortex and posterior cingulate compared to control subjects (Marx et al., 2006b), a finding that might reflect an adaptive and/or compensatory response. It was therefore hypothesized that PG levels would be reduced in parietal cortex and posterior cingulate in subjects with schizophrenia who died by suicide compared to subjects with schizophrenia who died by other causes.

PG, DHEA, and ALLO levels were determined by gas chromatography/mass spectrometry preceded by high performance liquid chromatography purification and analyzed non-parametrically using the Mann-Whitney U test statistic. The results are summarized in Table 2.

TABLE 2
Neuroactive steroid Levels (median) in
Parietal Cortex and Posterior Cingulate

| | | | | | | | |
|---|---|---|---|---|---|---|---|
| Disorder Grouping | n | PG | p* | DHEA | p* | ALLO | p* |

Median Neuroactive Steroid Levels in Parietal Cortex (ng/g)

| | | | | | | | |
|---|---|---|---|---|---|---|---|
| Schizophrenia | | | | | | | |
| Suicide | 4 | 19.01 | | 7.46 | | 6.62 | |
| No Suicide | 11 | 41.86 | 0.04 | 16.15 | 0.47 | 7.16 | 0.84 |
| Schizophrenia and | | | | | | | |
| Bipolar Disorder | | | | | | | |
| Combined | | | | | | | |
| Suicide | 13 | 23.38 | | 10.19 | | 6.51 | |
| No Suicide | 17 | 52.57 | 0.02 | 17.55 | 0.45 | 7.95 | 0.32 |

Median Neuroactive Steroid Levels in Posterior Cingulate (ng/g)

| | | | | | | | |
|---|---|---|---|---|---|---|---|
| Schizophrenia | | | | | | | |
| Suicide | 4 | 16.47 | | 9.84 | | 6.55 | |
| No Suicide | 11 | 25.06 | 0.21 | 16.81 | 0.22 | 7.44 | 0.95 |
| Schizophrenia and | | | | | | | |
| Bipolar Disorder | | | | | | | |
| Combined | | | | | | | |
| Suicide | 13 | 18.63 | | 12.14 | | 6.98 | |
| No Suicide | 17 | 34.41 | 0.06 | 16.81 | 0.17 | 8.46 | 0.93 |

*Mann-Whitney U test statistic

As shown in Table 2, the median .PG levels in parietal cortex were significantly lower in subjects with schizophrenia who committed suicide (19.01 ng/g; n=4) compared to subjects with schizophrenia who died of other causes (41.86 ng/g; n=11; p=0.04). Median PG levels in posterior cingulate were not significantly different in subjects with schizophrenia who died by suicide (p=0.21). When subjects with schizophrenia and bipolar disorder were combined, median PG levels were significantly lower in parietal cortex in subjects who died by suicide (23.38 ng/g; n=13) compared to subjects who died of other causes (52.57 ng/g; n=17; p=0.02), and also tended to be lower in posterior cingulate in subjects who committed suicide in this combined group (p=0.06).

PG levels are significantly reduced in parietal cortex in subjects with schizophrenia who died by suicide compared to subjects with schizophrenia who died of other causes. PG levels are also reduced in parietal cortex (significantly) and posterior cingulate (trend) in subjects who died by suicide compared to those who died of other causes when subjects with schizophrenia and bipolar disorder are combined. PG levels in subjects with schizophrenia are higher in both parietal cortex and posterior cingulate compared to control subjects.

Since PG levels in parietal cortex within the schizophrenia group are significantly higher in subjects who did not die of suicide, it is possible that PG elevations in schizophrenia represent an adaptive or compensatory response. A report that decreased CSF PG levels are associated with depressive symptoms potentially supports this possibility (George et al., 1994).

PG is markedly increased following clozapine administration in rodents (Marx et al., 2006b). It is therefore possible that clozapine-induced PG elevations contribute to its superior clinical efficacy and therapeutic actions on suicidal behaviors.

PG also demonstrates actions on learning and memory in rodents (Flood et al., 1992) and impacts the neuronal cytoskeleton (Hsu et al., 2006; Fontaine-Lenoir et al., 2006). PG sulfate enhances acetylcholine release (Vallee et al., 1997; Darnaudery et al., 1998, 2002). It is possible that this neuroactive steroid plays a role in the pathophysiology of suicide in schizophrenia and bipolar disorder. PG thus represents a logical target for pharmacological intervention in schizophrenia and bipolar disorder.

### Example 3

### Neuroactive Steroid Levels in Parietal Cortex and Posterior Cingulate of Subjects with and without Psychiatric Diagnoses

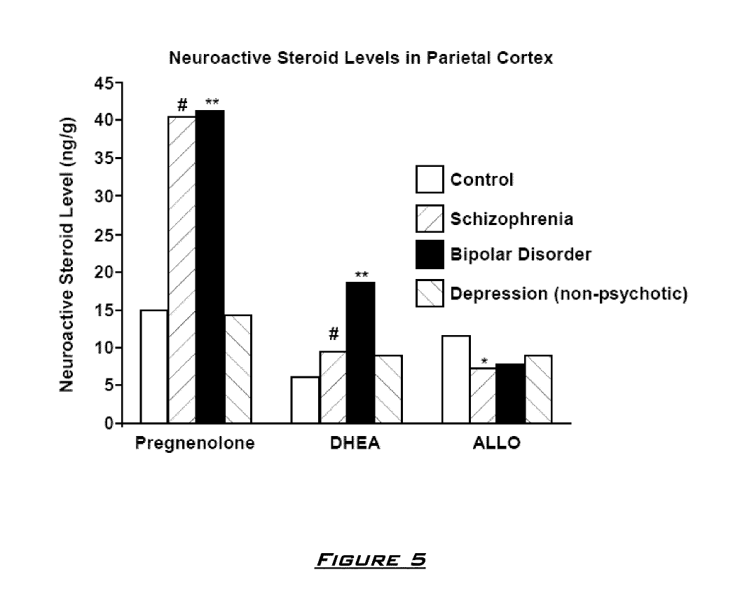

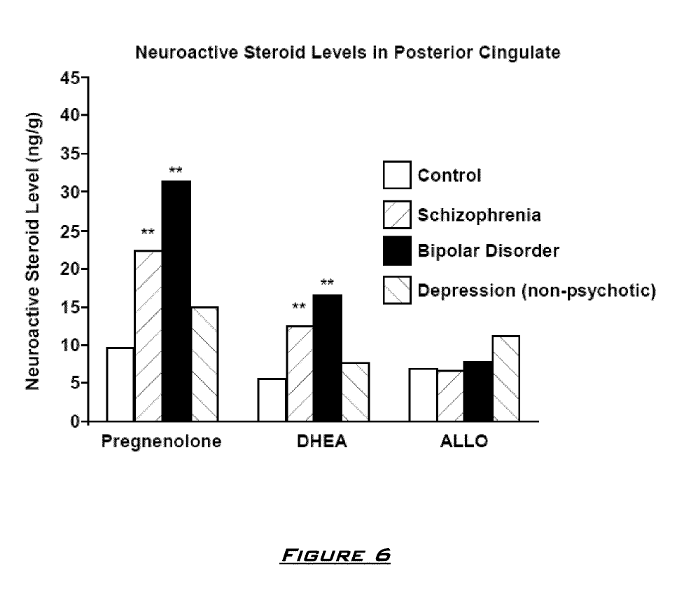

Median neuroactive steroid levels in parietal cortex and in the posterior cingulate in control subjects without a psychiatric diagnosis and in subjects with schizophrenia, bipolar disorder, and depression (non-psychotic) were determined using the methods disclosed in EXAMPLE 2. The results are summarized in FIGS. 5 and 6.

PG levels (log transformed) were significantly higher in parietal cortex tissue from subjects with bipolar disorder compared to control subjects (ANOVA p=0.0046; df 3,56; F =4.844; post-hoc Dunnett **p<0.01 for the bipolar disorder group, n=15). PG levels also tended to be higher in the schizophrenia group, but this finding was reduced to a trend in this brain region (post-hoc Dunnett #p=0.06, n=15).

DHEA levels (log transformed) were significantly higher in parietal cortex tissue in subjects with bipolar disorder compared to control subjects (ANOVA p=0.0087; df 3,56; F =4.272; post-hoc Dunnett **p<0.01 for the bipolar disorder group, n=15). DHEA levels also tended to be higher in the schizophrenia group, but this finding was reduced to a trend in this brain region (post-hoc Dunnett #p=0.06, n=15).

ALLO levels (log transformed) tended to be lower in parietal cortex tissue from subjects with schizophrenia compared to control subjects (ANOVA p=0.0911; df 3,56; F=2.263; post-hoc Dunnett *p=0.04 for the schizophrenia group, n=15).

### Example 4

### Neuroactive Steroid Levels in Temporal Cortex of Alzheimer's Disease Subjects

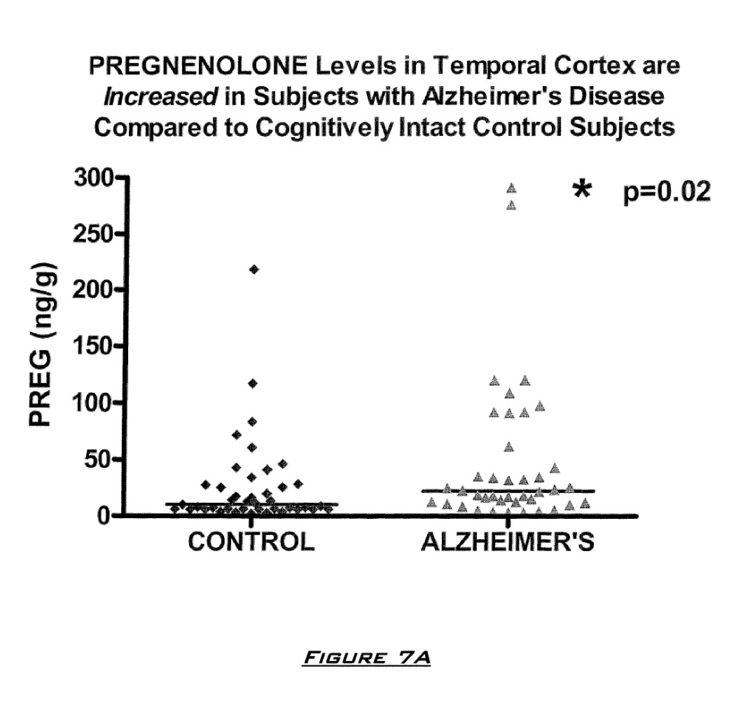

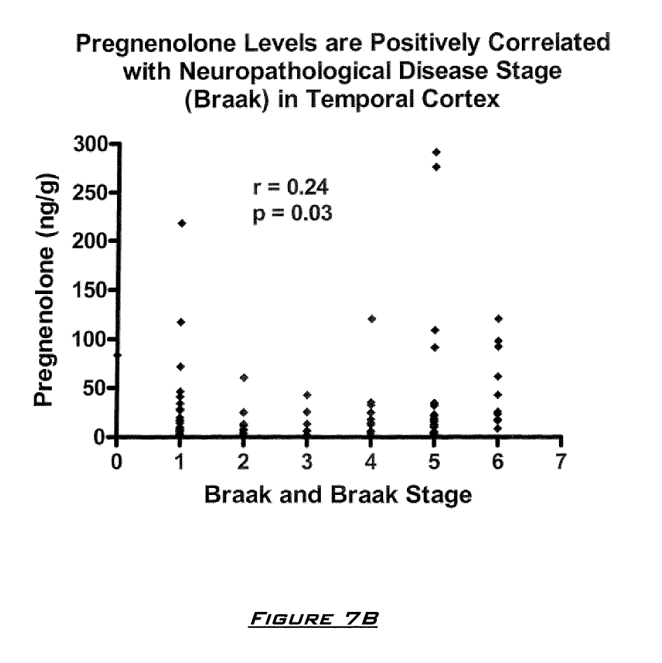

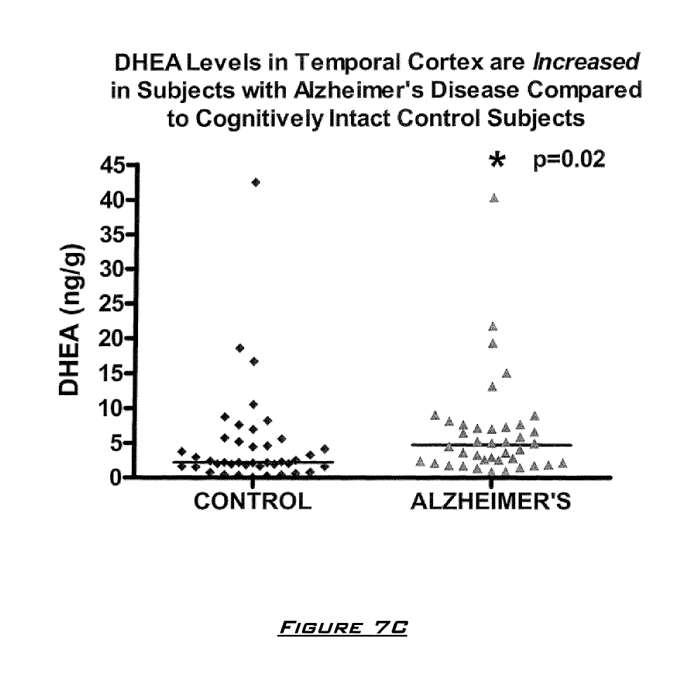

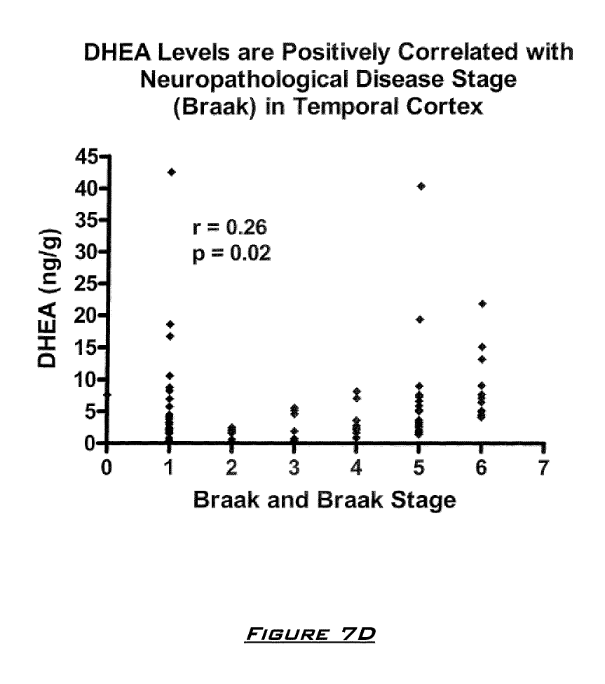

PG, DHEA, and ALLO levels were also examined in the temporal cortex of Alzheimer's disease subjects and compared to levels in control subjects. The results are summarized in FIGS. 7A-7F.

PG levels in the temporal cortex of subjects with Alzheimer's disease were significantly increased compared to levels in temporal cortex of cognitively intact subjects (p=0.02), and were positively correlated with neuropathological disease stage (Braak method; Braak & Braak, 1991; r=024; p=0.03). DHEA levels in the temporal cortex of subjects with Alzheimer's disease were also significantly increased compared to levels in temporal cortex of cognitively intact subjects (p=0.02), and were also positively correlated with neuropathological disease stage (Braak method; Braak & Braak, 1991; r=0.26; p=0.03).

On the contrary, ALLO levels in the temporal cortex of subjects with Alzheimer's disease were significantly decreased compared to levels in temporal cortex of cognitively intact subjects (p=0.0002), and were inversely correlated with neuropathological disease stage (Braak method; Braak & Braak, 1991; r=−0.38; p=0.0004).

### Example 5

### ALLO Levels in Temporal Cortex Are Associated with APOE 4 Allele Status

APOE is the major cholesterol transporter in the brain, and a number of other genes involved in cholesterol metabolism appear to be relevant to Alzheimer's disease. Since cholesterol is a precursor to pregnenolone and other neuroactive steroids, alterations in cholesterol metabolism in AD might have implications for neuroactive steroid regulation.

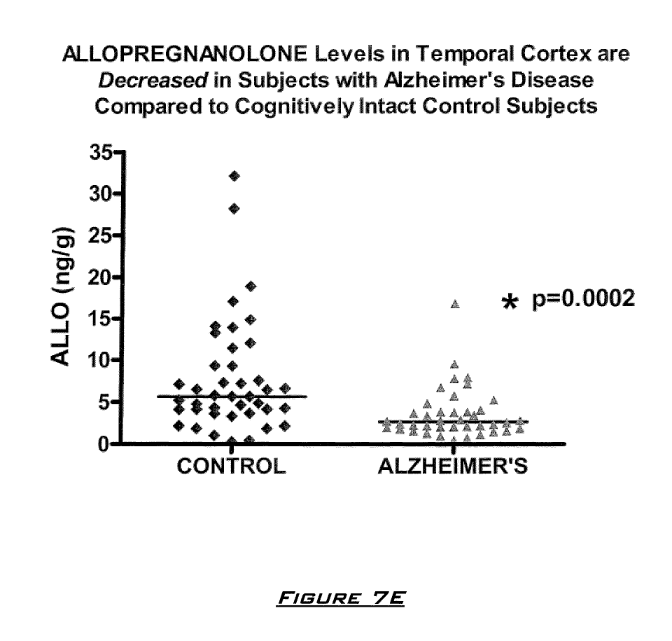

APOE status for all subjects contributing brain tissue specimens in the Duke Alzheimer's Disease Research Center brain collection was determined postmortem. ALLO levels in temporal cortex were significantly decreased (p=0.04) in subjects carrying an APOE 4 allele (see FIG. 8), suggesting that subjects that are heterozygous or homozygous for an APOE 4 allele might be predisposed to having reduced temporal cortex ALLO levels. Interestingly, the APO 4 locus on human chromosome 19 has been linked to a late-onset form of Alzheimer's disease (Saunders et al., 1993; Strittmatter et al., 1993).

### Example 6

### Neuroactive Steroid Levels in Prefrontal Cortex in Alzheimer's Disease Subjects

Neuroactive steroid (NS) levels were also examined in prefrontal cortex (PFC) of subjects with Alzheimer's disease and compared to NS levels in prefrontal cortex of cognitively intact control subjects as reported in Marx et al., 2006c. Briefly, frozen right-hemisphere PFC tissue samples from 14 male subjects with AD and 15 cognitively intact male control subjects were analyzed for NS (ALLO, PG, and DHEA) by the highly sensitive and specific gas chromatography/mass spectrometry (GC/MS) method preceded by HPLC purification described hereinabove. The primary outcomes (NS levels in AD vs. cognitively intact control subjects) were analyzed non-parametrically by Mann Whitney Utest statistic. Correlational analyses (NS levels vs. Braak and Braak neuropathological disease stage) were also assessed non-parametrically and Spearman correlation coefficients were determined. Both AD subjects and control subjects were included in the correlational analyses, because cognitively intact control subjects can meet neuropathological criteria for early Braak stages (potentially reflecting the earliest stages of AD or predisposition to developing AD in the absence of detectable clinical symptomatology); p values less than or equal to 0.05 were considered to be statistically significant.

ALLO levels in PFC were significantly reduced in subjects with AD compared with cognitively intact control subjects (median ALLO levels 2.50 ng/g vs. 5.59 ng/g, respectively; Mann-Whitney Utest statistic p=0.02). ALLO levels were inversely correlated with neuropathological disease stage (Braak method; Spearman r=−0.49; p=0.007).

DHEA levels, on the other hand, were significantly higher in AD compared with cognitively intact control subjects (median DHEA levels 2.61 ng/g vs. 1.12 ng/g, respectively; Mann-Whitney U test statistic p=0.01). The DHEA levels tended to be positively correlated with Braak stage (Spearman r=0.32; p=0.09).

Although PG levels tended to be higher in the AD group, this result did not achieve statistical significance (median PG levels 20.50 ng/g vs. 8.61 ng/g, respectively; Mann-Whitney U test statistic p=0.07). PG levels were not significantly correlated with Braak stage (Spearman r=0.28; p=0.14).

### Example 7

### Neuroactive Steroid Levels in Cerebrospinal Fluid

In an effort to determine if neuroactive steroid (NS) levels in brain correlated with neuroactive steroid levels in cerebrospinal fluid (CSF), the levels of PG and DHEA were also determined in CSF isolated from Alzheimer's disease (AD) subjects. CSF was available from 41 out of the 81 subjects that were tested for PG and DHEA levels in the temporal cortex, which included 16 cognitively intact subjects (controls) and 25 subjects with AD. The same GC/MS preceded by HPLC strategy discussed hereinabove was employed for determining NS levels in CSF.

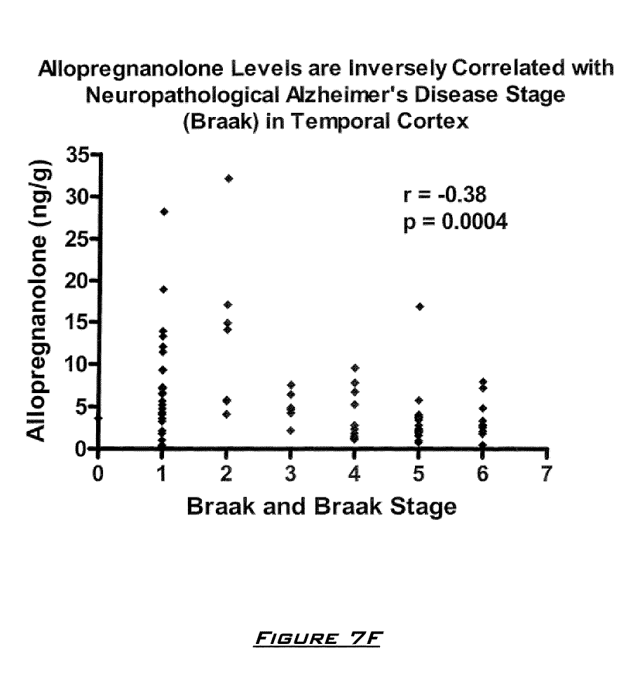

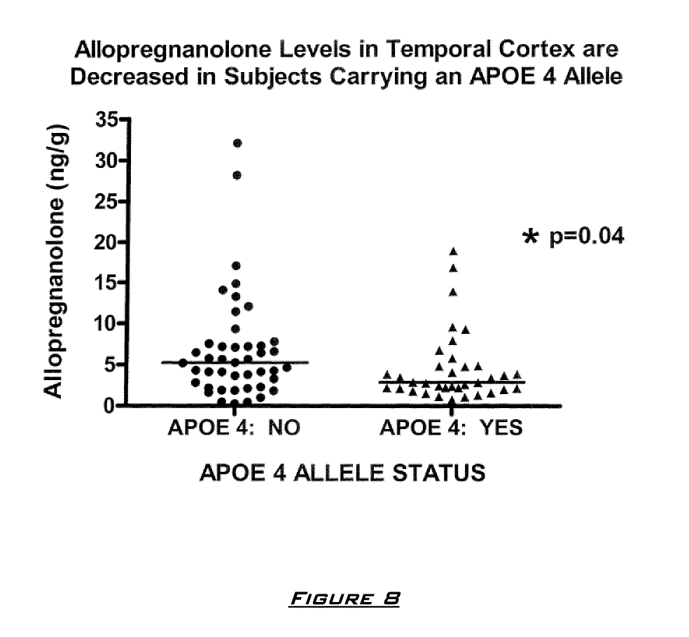

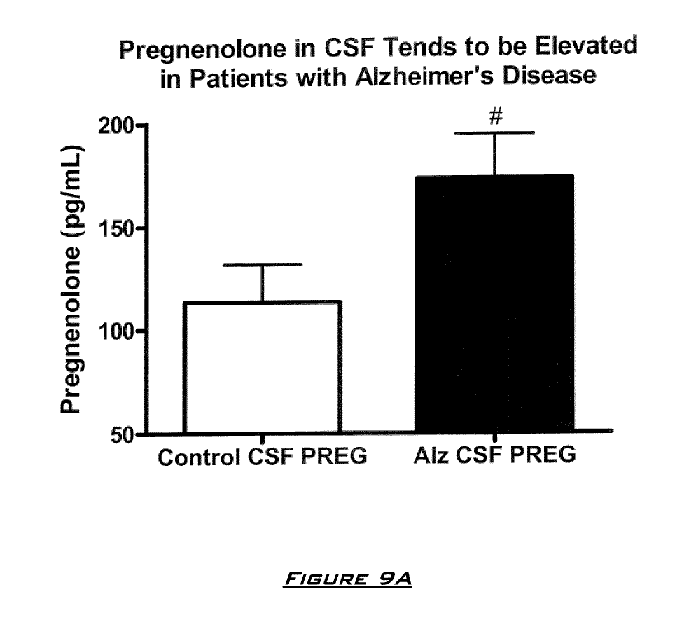

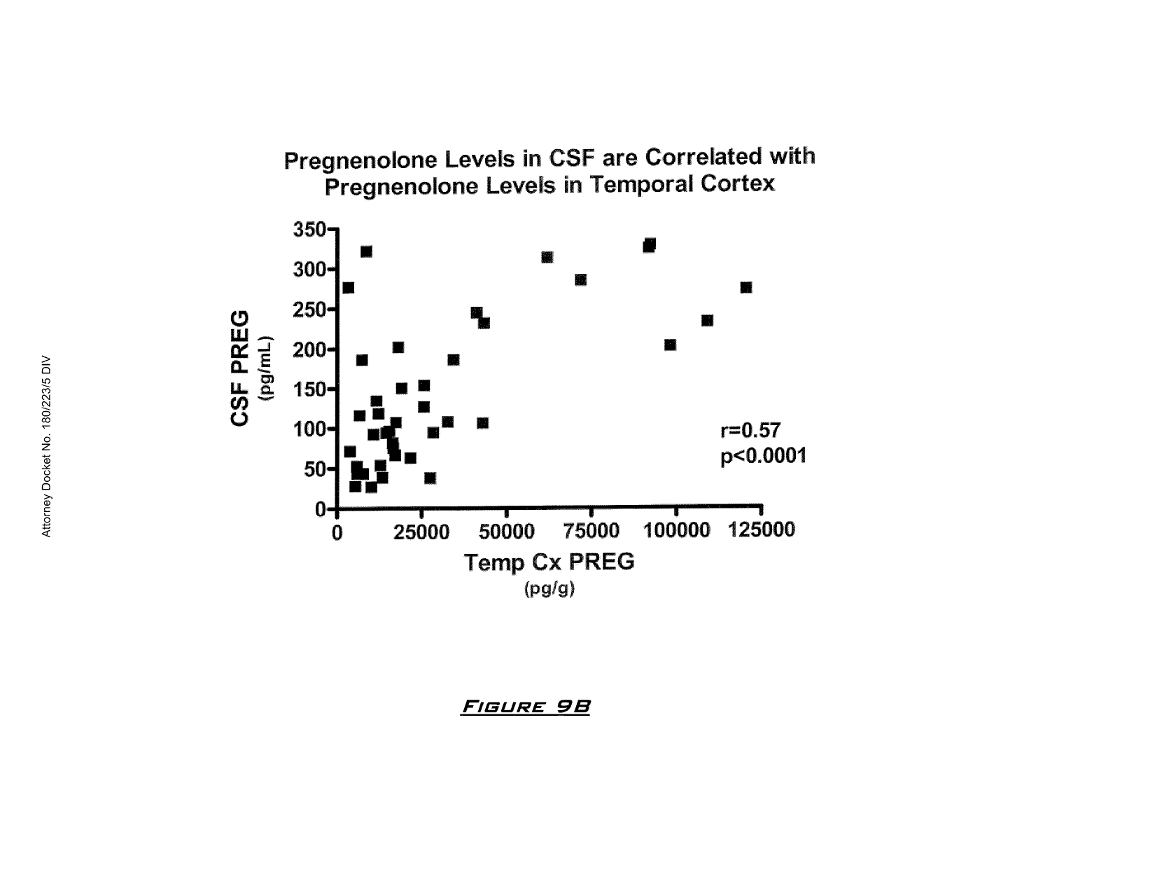

The results for PG are presented in FIGS. 9A-9C. PG levels, which were elevated in temporal cortex of subjects with AD, tended also to be elevated in the CSF of subjects with AD (p=0.10). CSF and temporal cortex PG levels were positively correlated (r=0.57; p<0.0001). PG levels, which in temporal cortex were positively correlated with Braak stage, also tended in CSF to be positively correlated with Braak stage (r=0.30; p=0.06).

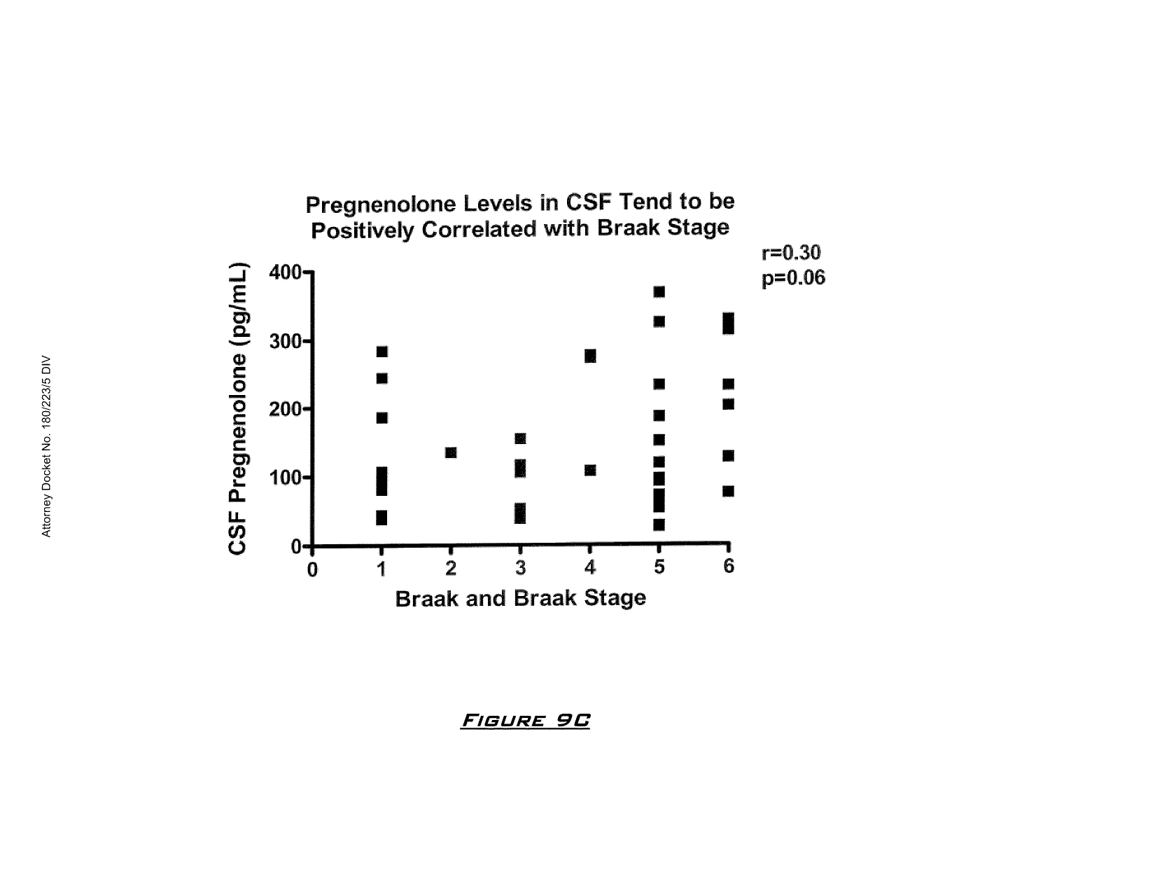

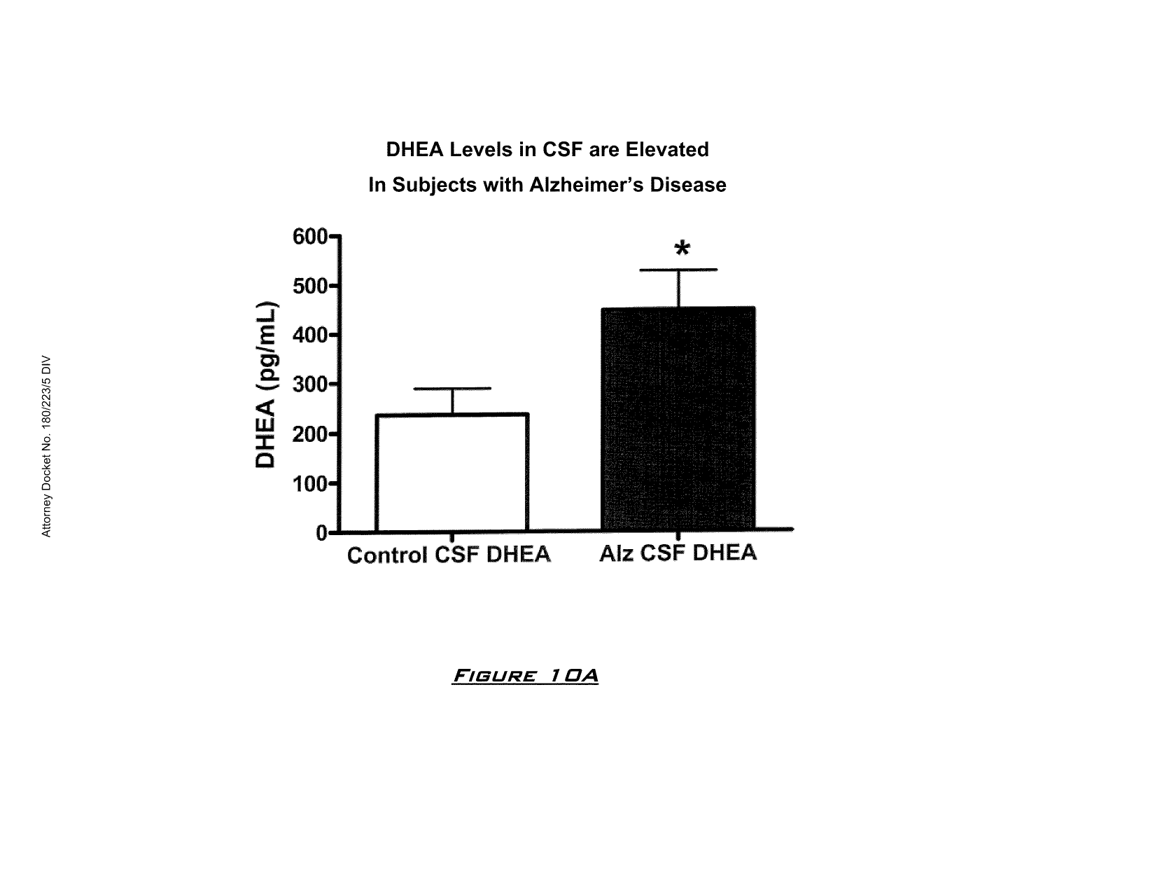

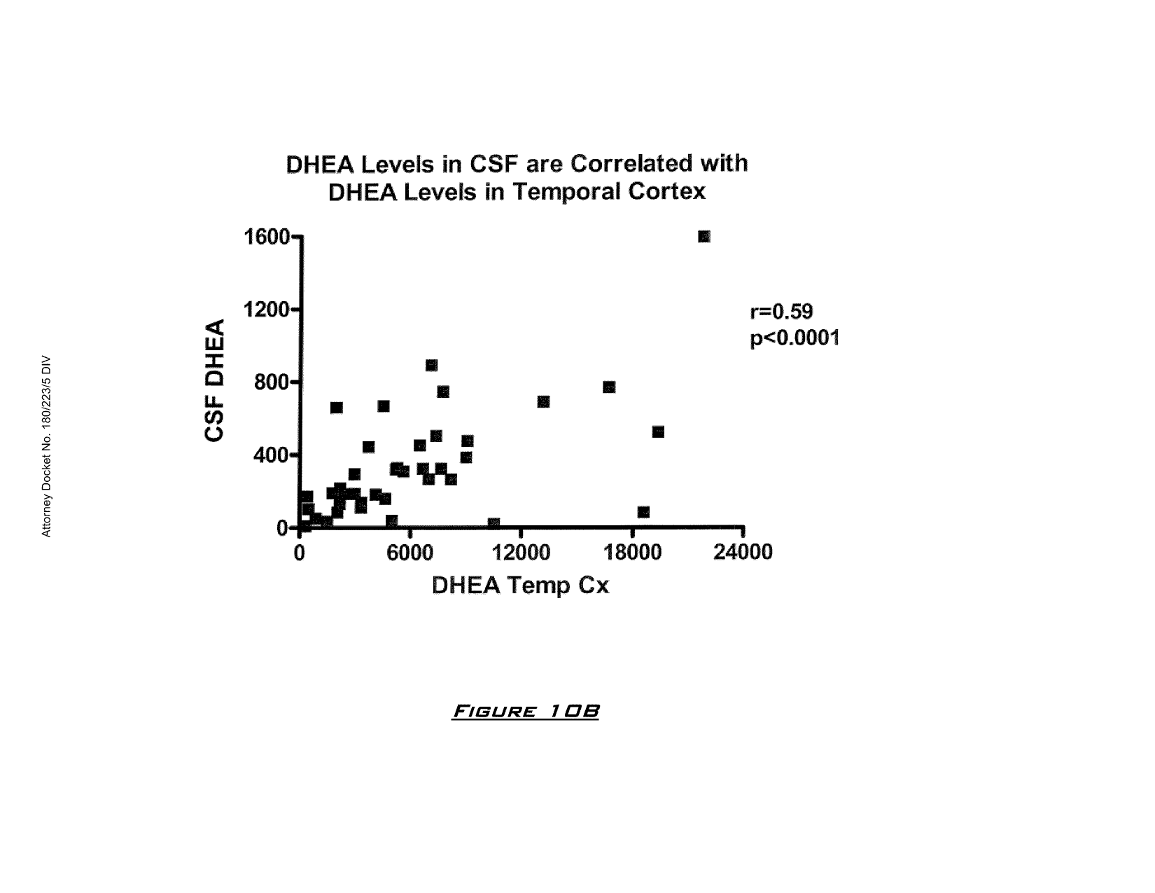

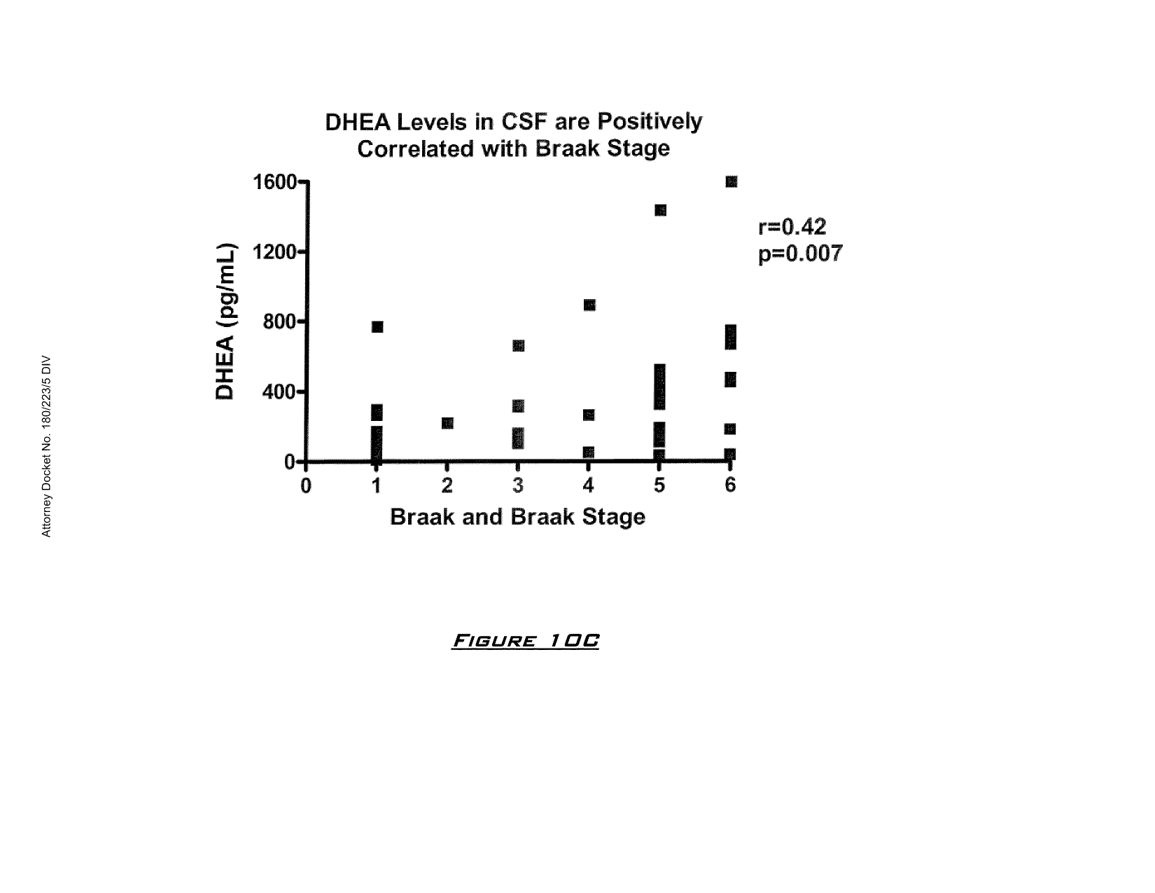

DHEA levels were also assayed in CSF, and the results are summarized in FIGS. 10A-10C. DHEA levels, which were elevated in temporal cortex, were also significantly elevated in subjects with AD (p=0.032). CSF and temporal cortex DHEA levels were also positively correlated (r=0.59; p<0.0001). Also similar to the case in temporal cortex, CSF DHEA levels were positively correlated with Braak stage (r=0.42; p=0.007).

### Example 8

### Neuroactive Steroids in Depression

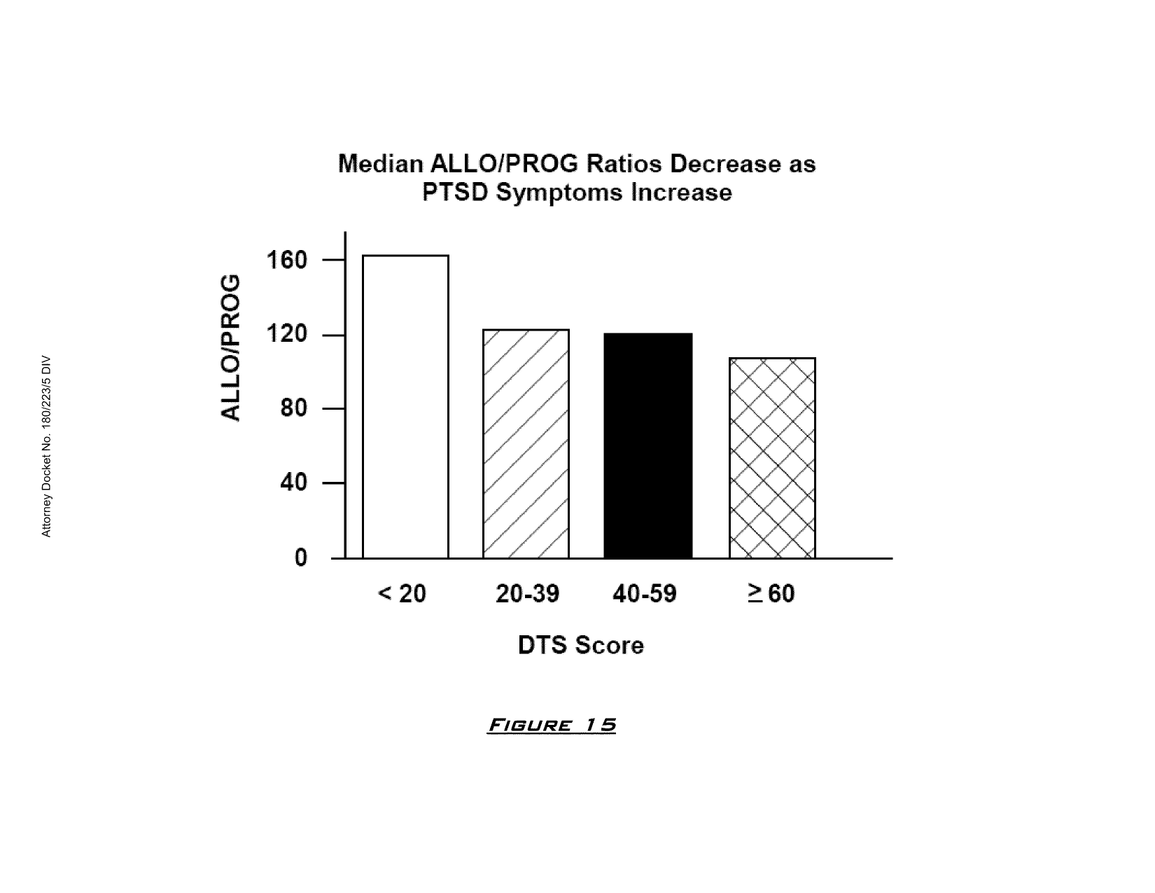

Fluoxetine elevates brain ALLO levels, and PG levels. In addition, it has been determined that ALLO predicts depressive symptoms as measured by the Beck Depression Inventory and the SCL-90 depression component in newly returning veterans who served in Iraq or Afghanistan (see EXAMPLE 13 and FIG. 16).

### Example 9

### Neuroactive Steroids in Post Traumatic Stress Disorder (PTSD)

Adult civilian outsubjects meeting Diagnostic and Statistical Manual of Mental Disorders, Fourth Edition (DSM-IV) criteria for chronic PTSD were enrolled in an open label sertraline (ZOLOFT®) trial, flexible dosing 50-200 mg per day, n=10, mean age 38.3 years. Several PTSD rating scales were utilized at baseline and following 15 weeks of sertraline treatment, including the Post-traumatic Diagnostic Scale (PDS), Short PTSD Rating Interview (SPRINT), Davidson Trauma Scale (DTS), and Structured Interview for PTSD (SIP), as well as the Self-Rated Improvement Scale (SRS) and Beck Depression Inventory (BDI). A resilience scale was also administered (Connor-Davidson Resilience Scale, CD-RISC).Serum was obtained at baseline prior to beginning sertraline and following 15 weeks of treatment, and frozen at −80° C. Linear regression analyses were performed and Pearson correlation coefficients were determined to investigate potential associations between symptom rating scales (PDS, SPRINT, DTS, SIP, SRS, CD-RISC) and neuroactive steroid levels.

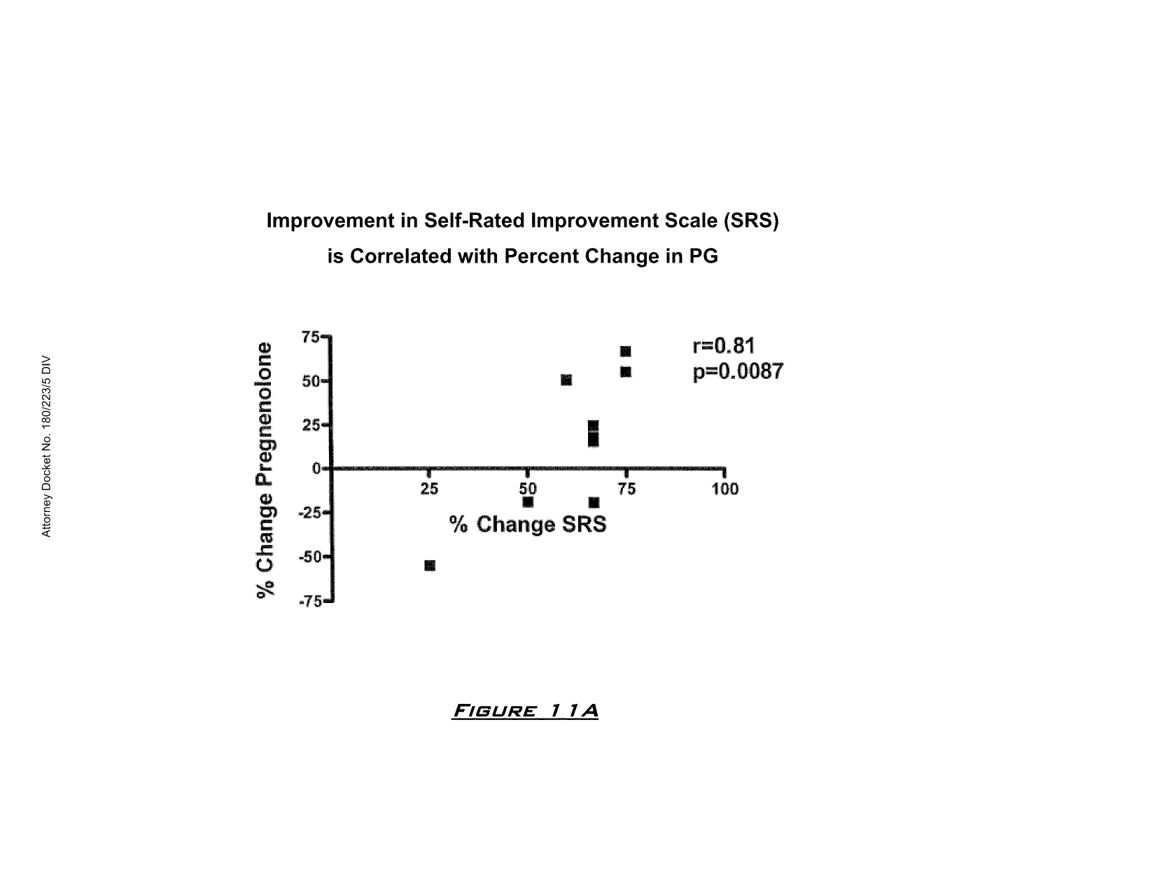

Changes in PG levels were positively correlated with improvements in the SRS and DTS scales (r=0.81; p=0.0087; and r=0.69; p=0.038, respectively) following treatment with sertraline (see FIGS. 11A and 11B). It is therefore possible that elevations in PG following treatment with this selective serotonin reuptake inhibitor (SSRI) contribute to its therapeutic efficacy, and these preliminary data support the possibility that PG augmentation can be helpful in the treatment of persistently symptomatic subjects with PTSD

Summarily, these preliminary findings regarding PG, DHEA, and ANDRO in subjects with PTSD following treatment with sertraline (ZOLOFT®, an SSRI) suggested that neuroactive steroids are linked to PTSD symptoms. Changes in PG were positively correlated with symptom improvement in subjects with PTSD following sertraline, suggesting that PG augmentation could be efficacious for PTSD core symptoms in addition to cognitive symptoms. Additionally, both DHEA and ANDRO were positively correlated with PTSD symptoms following sertraline administration. These data suggested that PG augmentation can also effectively reduce core symptoms of PTSD, and potentially ameliorate cognitive symptoms common to PTSD.

### Example 10

### Additional Studies on Neuroactive Steroids in PTSD

Based on the observation that PG increases in subjects with PTSD following sertraline predicted improvement in symptoms, a randomized controlled trial utilizing PG in subjects with PTSD targeting cognitive symptoms is performed to determine the following:
(1) if adding PG to ongoing SSRI treatment reduces cognitive symptoms in subjects with PTSD and/or if augmenting SSRIs with PG improves PTSD symptoms, depressive symptoms, and overall functioning and
(2) PG and PG metabolite levels to identify specific neuroactive steroid alterations that can contribute to clinical effects (including potential increases in DHEA, ALLO, and other neuroactive steroids) using mass spectrometry techniques and to characterize protein and small molecule changes.

With respect to the first goal, a First Hypothesis to be tested is that adding PG to ongoing SSRI treatment reduces cognitive symptoms as measured by improved scores on the BAC-A (the primary outcome measure for this hypothesis), as well as the CPT and Trail Making Tests A+B (secondary outcome measures for this hypothesis) in subjects with PTSD. A Second Hypothesis to be tested is that adding PG to ongoing SSRI treatment will improve PTSD symptoms as measured by the CAPS (the primary outcome measure for this hypothesis), as well as PTSD symptoms as measured by the PCL, depressive symptoms as measured by the BDI-II, and overall functioning as measured by the Connor-Davidson Resilience Scale and the Heinrich-Carpenter Quality of Life questionnaire (secondary outcome measures for this hypothesis).

### Experimental Design and Methods

Subjects. Thirty veterans between the ages of 18-65 are enrolled. Both male and female subjects and all ethnic groups are eligible to participate in this study. All subjects have a DSM-IV diagnosis of PTSD. Subjects are recruited from the Durham VA Medical Center (Durham, N.C.).

Relevant Prior Literature and Group Size. An n=15 per group is chosen for this augmentation study to SSRI treatment-as-usual (30 subjects total; 15 subjects randomized to PG, 15 subjects randomized to placebo) based on a careful review of the existing literature and the power analysis provided below.

A prior placebo-controlled double-blind study utilizing olanzapine as an augmentation agent to SSRI treatment-as-usual in subjects with PTSD determined that olanzapine augmentation was associated with a statistically significantly greater reduction than placebo in specific measures of traumatic stress, depressive, and sleep disorder symptoms (Stein et al., 2002). The total number of subjects randomized in this study was 19 (n=9-10 per group). Despite small group sizes of 9-10 per group, significant findings were detected (Stein et al., 2002).

For the current PG augmentation to SSRI-as-usual study, 15 subjects per group, or 50% more subjects than the preceding study, are enrolled. Along similar lines, a neuroactive steroid augmentation study also demonstrated significant results in a placebo-controlled double-blind investigation utilizing DHEA in subjects with schizophrenia with a sample size of 12-15 per group (Strous et al., 2003). Two important studies have therefore used a similar placebo-controlled double-blind augmentation design enrolling 15 or fewer subjects per group to the one proposed in this investigation.

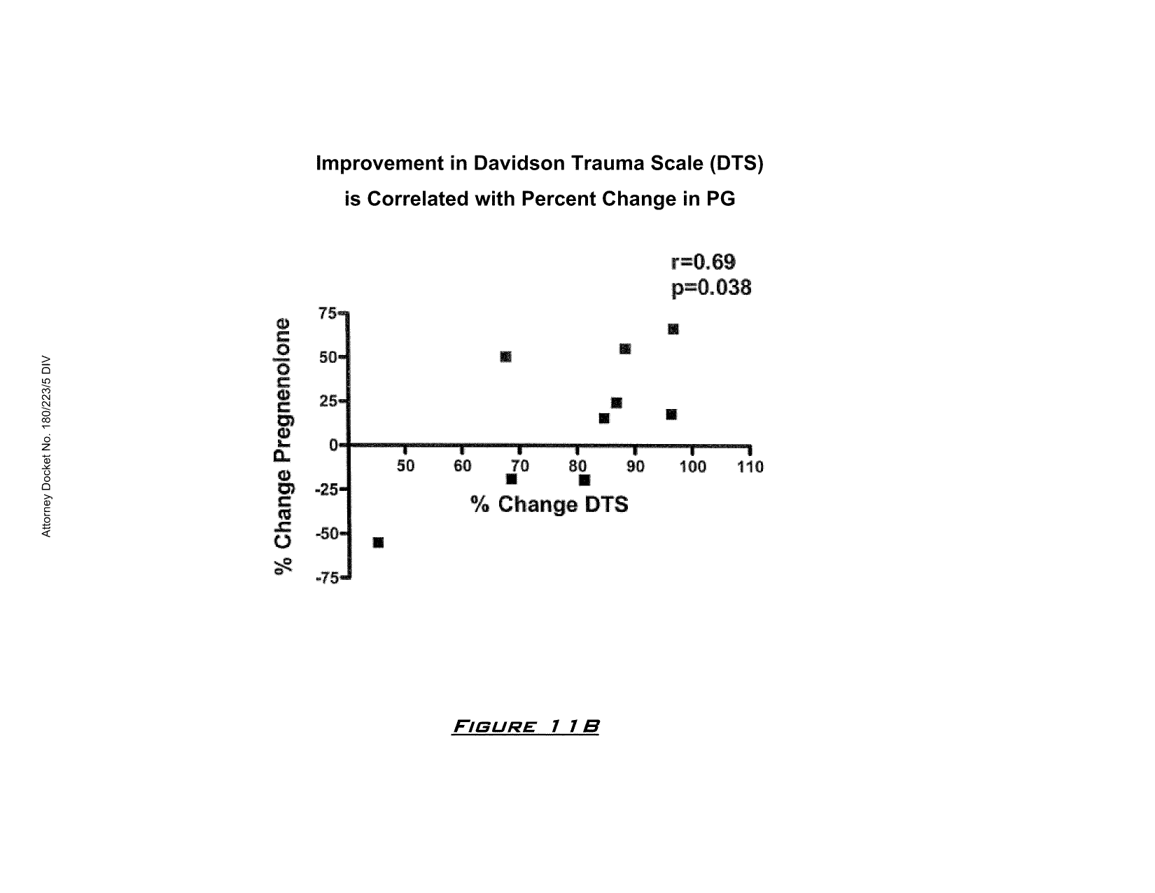

Power Analysis. The following power analysis assumes a randomized controlled parallel group design. In this study, 30 participants are randomized to either a treatment (PG) or placebo group with equal probability. The two study arms are concurrent, (i.e., the active and control treatments occur in the same time period). The primary outcomes for the study (i.e., BAC-A and CAPS) are treated as continuous measures. According to the method of David Schoenfeld (MGH Biostatistics, Massachusetts General Hospital, Boston, Massachusetts), FIG. 12 illustrates the statistical power associated with various detectable differences between study groups. The difference is expressed as a multiple of the standard deviation of the outcome measure. For example, in the above case, if the standard deviation is 10, then this study has 80% power to detect a group difference of 12. Thus, the absolute detectable difference at 80% power is a function of the standard deviation of the outcome variable of interest, given the above assumptions.

From the above, a total of 30 subjects enter this two treatment parallel-design study. The probability is 80 percent that the study detects a treatment difference at a two sided 5.000 percent significance level, if the true difference between the treatments is 1.07 times the standard deviation.

Power for the First Hypothesis: The detectable difference between the intervention and control group at 80% power for BAC-A (estimated mean from literature =1.49) depends on the unknown standard deviation as depicted in Table 3.

TABLE 3
Power Analysis for the First Hypothesis

| Standard Deviation | Absolute Detectable | Relative Detectable |
|---|---|---|
| for BAC-A | Difference | Difference |
| 0.20 | 0.21 | 14% |
| 0.30 | 0.32 | 21% |
| 0.40 | 0.43 | 29% |
| 0.50 | 0.54 | 36% |
| 0.60 | 0.64 | 43% |

Thus, an 80% power to detect a 21% difference between intervention and control groups is present if the standard deviation for BAC-A is 0.30.

Power for Second Hypothesis: The detectable difference between the intervention and control group at 80% power for CAPS (estimated mean from literature =75) depends on the unknown standard error as depicted in Table 4.

TABLE 4
Power Analysis for the Second Hypothesis

| Standard Deviation | Absolute Detectable | Relative Detectable |
|---|---|---|
| for CAPS | Difference | Difference |
| 5 | 5 | 7% |
| 10 | 11 | 15% |
| 15 | 16 | 21% |
| 20 | 21 | 28% |
| 25 | 27 | 36% |

Thus, an 80% power to detect a 21% difference between intervention and control groups is present if the standard deviation for CAPS is 15.

Recruitment. Study subjects who are receiving outsubject care are recruited from the Durham VA Medical Center. A subject who is judged likely meet all of the inclusion criteria and none of the exclusion criteria meets with the research nurse and a research physician to discuss the research protocol, and to determine if the subject is capable of providing informed consent. A member of the research team then meets with the subject to answer any questions and to obtain informed consent. Subjects who are eligible for the study and give their written informed consent proceed to a 1 hour screening visit. It is estimated that subsequent visits (every 2 weeks for the duration of the study) will each require about 2-4 hours of subject time.

Risks/Benefit Assessment. Subjects are not tapered from their current stable PTSD treatment regimen; PG is only “added on” to treatment-as-usual. Notification has recently been received from the FDA permitting this study to proceed: the assigned IND number for this study is 73,099. PG has been well-tolerated at the doses proposed in this study. Previously reported uncommon adverse reactions include headache, rash, insomnia, and stomach upset, and one report of palpitations in the existing literature. A baseline, 6-week, and 10-week EKG is performed to closely monitor subjects and perform a Chem 7 at each visit to confirm the absence of rhythm irregularities or electrolyte disturbance. Blood draws at each visit are minimal risk. Possible side effects of drawing blood include bruising, bleeding, or pain at the injection site, and (rarely) fainting and infection.

Pregnenolone. Douglas Laboratories (Pittsburgh, Pa.) has supplied PG and matching placebo. They have guaranteed PG purity. Active capsules are 50 mg; matched placebo is manufactured by the same company.

All pills are supplied to subjects in two-week, twice-a-day bottles. Subjects are told that they might get placebo, that the dose of medication might vary over time and change every two weeks, and that they might not receive any active medication.

Study Design, Methods, and Procedures.

This study tests the efficacy of augmenting current SSRI treatment with the neuroactive steroid PG on both cognitive and core PTSD symptoms in subjects with PTSD. The proposed design is a randomized, placebo-controlled, double-blind, trial of adjunctive treatment of a stable outsubject SSRI regimen (no change in SSRI dose for ≦4 weeks). The total study duration is 10 weeks. Following a 2-week placebo-only lead-in period, 30 subjects are randomly assigned to one of two groups. Of these subjects, 15 subjects receive PG and 15 subjects receive placebo for 8 weeks. PG levels and PG metabolite levels (including DHEA and the GABAergic neuroactive steroid ALLO) are determined to investigate if increases in PG or other neuroactive steroid metabolites correlate with therapeutic efficacy.

Proteins and small molecules are also investigated. Subjects are screened to determine if they meet inclusion criteria. Screening includes a psychiatric assessment, physical examination, EKG, baseline lab tests, and urinalysis to assess general physical health. A urine toxicology screen is also performed. Several laboratory studies are performed at weeks 0, 2, 4, 6, 8, and 10 as set forth in the Schedule of Events in Table 5.

TABLE 5
Schedule of Events

| | PG Augmentation Study | | | | | | | | | | |
|---|---|---|---|---|---|---|---|---|---|---|---|
| | Visit 1: | Phone | Visit 2: | Phone | | Phone | | Phone | | Phone | Visit 6: |
| | Screening | check-in | Baseline | check-in | Visit 3 | check-in | Visit 4 | check-in | Visit 5 | check-in | Final |
| | Week | Week | Week | Week | Week | Week | Week | Week | Week | Week | Week |
| Procedures | 0 | 1 | 2 | 3 | 4 | 5 | 6 | 7 | 8 | 9 | 10 |
| Informed | X | | | | | | | | | | |
| Consent | | | | | | | | | | | |
| Inclusion/ | X | | | | | | | | | | |
| exclusion | | | | | | | | | | | |
| Demographics | X | | | | | | | | | | |
| Med. & | X | | | | | | | | | | |
| Psych. Hist. | | | | | | | | | | | |
| Psych Diag. | X | | | | | | | | | | |
| (MINI) | | | | | | | | | | | |
| Vital Signs | X | | X | | X | | X | | X | | X |
| Physical Exam | X | | | | | | | | | | X |
| Preg. Test | X | | | | | | X | | | | X |
| (females) | | | | | | | | | | | |
| EKG | X | | | | | | X | | | | |
| Chem 7 | X | | X | | X | | X | | X | | X |
| CBC | X | | X | | X | | X | | X | | X |
| GI Panel | X | | X | | X | | X | | X | | X |
| Lipid Panel | X | | | | | | X | | | | |
| TSH | X | | | | | | | | | | |
| Prolactin | X | | | | | | | | | | |
| PG, other NS, | X | | X | | X | | X | | X | | X |
| and Protein | | | | | | | | | | | |
| Urine drug | X | | | | | | X | | | | X |
| screen | | | | | | | | | | | |
| Urinalysis | X | | | | | | | | | | X |
| BAC-A | | | X | | | | X | | | | X |
| CPT | | | X | | | | X | | | | X |
| Trail Making | | | X | | | | X | | | | X |
| A + B | | | | | | | | | | | |
| WRAT | X | | | | | | | | | | X |
| CAPS | | | X | | | | X | | | | X |
| Connor-Davidson | | | X | | | | X | | | | X |
| Resilience Scale | | | | | | | | | | | |
| PCL | X | | X | | X | | X | | X | | X |
| BDI-II | | | X | | | | X | | | | X |
| Heinrich-Carpenter | | | X | | | | X | | | | X |
| Qual. of Life | | | | | | | | | | | |
| CGI | | | X | | X | | X | | X | | X |
| Hillside AE | | | X | X | X | X | X | X | X | X | X |
| Scale | | | | | | | | | | | |
| Concomitant | Placebo | Placebo | PG or | PG or | PG or | PG or | PG or | PG or | PG or | PG or | |
| Medications | | | Placebo | Placebo | Placebo | Placebo | Placebo | Placebo | Placebo | Placebo | |
| PG/Placebo Dose* | 0 | 0 | 50 | 50 | 150 | 150 | 250 | 250 | 250 | 250 | |
| Total daily | 0 | 0 | 100 | 100 | 300 | 300 | 500 | 500 | 500 | 500 | |
| dose (mg) | | | | | | | | | | | |

*Dose is in milligrams, administered peroral (PO), twice a day (BID)

Blood is drawn at baseline and weekly thereafter to monitor PG and other neuroactive steroid metabolite levels. Thirty subjects are randomly chosen to receive either PG or placebo (n=15 per group) and evaluated weekly for 10 weeks (Screening Visit +Weeks 1-10). Subjects come into the lab for the screening visit and every other week thereafter to complete the evaluations listed in the schedule of events. Between lab visits, subjects are contacted by telephone to complete the Hillside Adverse. Event Scale. Those subjects randomized to PG receive this neuroactive steroid on the following schedule:
Placebo lead-in phase 0 mg in two doses (0 mg, PO, BID) for 2 weeks; then
PG: 100 mg in divided doses (50 mg, PO, BID) for 2 weeks; then
PG: 300 mg in divided doses (150 mg, PO, BID) for 2 weeks; then
PG: 500 mg in divided doses (250 mg, PO, BID) for 4 weeks.

These doses were chosen after carefully reviewing prior dosing strategies in the existing literature, which were tolerated without significant side effects and produced maximal efficacy. Although recent human studies are very few, a single dose of PG 175 mg approximately doubled serum PG levels over the course of 5-8 hours (Roberts, 1995; Morley et al., 1997). Since PG levels decrease by approximately 60% with age (Roberts, 1995), the dosing strategy of 500 mg in the last 4 weeks of the protocol is anticipated to produce PG levels that either achieve or approximately double those observed in young adulthood. Possible adverse side effects are evaluated each week and at week 10 or at study termination (if a subject is withdrawn or chooses to leave the study).

Inclusion Criteria.
1. 18-65 years of age, any ethnic group, either sex;
2. DSM-IV diagnosis of PTSD by MINI;
3. No change in SSRI for ≦4 weeks;
4. No anticipated need to alter any psychotropic medications for the 10-week duration of the study; and
5. Ability to fully participate in the informed consent process, or have a legal guardian able to participate in the informed consent process.

Exclusion Criteria.

Exclusion Criteria
1. Unstable medical or neurological illness, including seizures, CVA, prostate or breast cancer (since PG supplementation could theoretically increase downstream steroid metabolites);
2. Use of oral contraceptives or other hormonal supplementation such as estrogen (although early studies suggested no effects on menstrual cycle, alterations in downstream metabolites of PG such as progesterone or estradiol could theoretically impact efficacy of oral contraceptives and estrogen replacement);
3. Significant suicidal or homicidal ideation;
4. Concomitant medications for medical conditions are addressed on a case-by-case base to determine if exclusionary;
5. Current DSM-IV diagnosis of bipolar disorder, schizophrenia, or other psychotic disorder, or cognitive disorder due to a general medical condition; history of substance dependence within the last 3 months;
6. Female subjects who are pregnant or breast-feeding;
7. Known allergy to study medication; and
8. Drugs with a narrow therapeutic index (e.g., thioridazine, mesoridazine, ziprasidone, clozapine, etc.) are excluded; subjects taking these agents are not eligible for this study.

### Scale Descriptions

Medical and Psychiatric History. Subjects are asked to list all medical and psychiatric conditions they have been diagnosed with and the approximate time since the diagnosis.

Psychiatric Diagnosis (MINI). The MINI was designed as a brief structured interview for the major Axis I psychiatric disorders in DSM-IV and ICD-10. Validation and reliability studies have been done comparing the MINI to the MINI-P for DSM-III-R and the CIDI (a structured interview developed by the World Health Organization for lay interviewers for ICD-10). The results of these studies show that the MINI has acceptably high validation and reliability scores, but can be administered in a much shorter period of time (mean 18.7±11.6 minutes, median 15 minutes) than the above referenced instruments. It can be used by clinicians, after a brief training session. Lay interviewers require more extensive training.

BAC-A. The primary neurocognitive assessment is the BAC-A. This battery is brief (about 50 minutes) and is devised for easy administration and scoring by non-psychologists. The BAC-A is specifically designed to measure treatment-related improvements and includes alternate forms. The BAC-A comprises brief assessments of executive functions, verbal fluency, attention, verbal memory, working memory, and motor speed. The BAC-A is very reliable, and a composite score can be generated that is sensitive to the cognitive deficits of affective disorders and anxiety disorders specifically. The BAC-A comprises the following assessments:

List Learning (Verbal Memory): Subjects view 15 words, then are asked to recall as many as possible. This is repeated five times. Measure: Number of words recalled per trial. Time: 7 minutes.

Digit Sequencing Task (Working Memory): Subjects are presented with clusters of numbers of increasing length. The experimenter asks the subject to list the numbers in order from lowest to highest. Measure: The number of correct responses. Time: 3 minutes.

Token Motor Task (Motor Speed): Subjects are given 100 plastic tokens and are asked to place them into a container as quickly as possible for 60 seconds. Measure: The number of tokens placed into the container. Time: 3 minutes.

Category Instances (Semantic Fluency): Subjects are asked to name as many words as possible within a given category within 60 seconds: Supermarket items.

Controlled Oral Word Association Test (Letter Fluency): In two separate trials, subjects are given 60 seconds to generate as many words as possible that begin with a given letter: “F” and “S.” Measure: Number of words generated per trial. Time: 5 minutes.

Tower Test (Executive Functions): Subjects look at two pictures simultaneously. Each picture shows three different colored balls arranged on 3 pegs, with the balls in a unique arrangement in each picture. The subject gives a total number of times the balls in one picture would have to be moved to make the arrangement of balls identical to that of the other, opposing picture. Measure: Number of correct responses. Time: 7 minutes.

Symbol Coding (Attention & Motor Speed): As rapidly as possible, subjects write numbers 1-9 as matches to symbols on a response sheet. Measure: Number of correct responses. Time: 3 minutes.

The BAC-A has all of the BACS tests plus two affectively-related tests: one that asks subjects to remember non-affective words (fruits and vegetables) and that asks subjects to remember affective words (e.g., lonely, killer). These tests and administration times are described below.

Verbal Memory and Learning.

Verbal Memory (7 minutes). Subjects are presented with 15 words and then asked to recall as many as possible. This procedure is repeated 5 times. Measures: verbal recall (number of words).

Affective Control.

Affective Interference Test (7 minutes). Subjects are presented with 20 affective and non-affective words and asked to recall as many as possible. This procedure is repeated 3 times and is followed by 2 cued-recall trials. Measures: verbal recall (number of affective and non-affective words).

Affective Interference Test-Delayed Recognition Task (2 minutes). 15-20 minutes after the Affective Interference Test subjects are asked whether certain affective and non-affective words were included in the previous word list. Measures: number of correct affective, correct non-affective, affective false-alarms, and non-affective false alarms.

Emotion Inhibition Test [Stroop] (4 minutes). Subjects are presented with sheets of paper with four columns of words (neutral or affective) or symbols in colored or black ink. They are asked to either read the words or name the colors of the ink going down the columns. They will get 30 seconds for each page. Measures: number of items correctly named in 30 seconds for each page. Key outcome measure is subjects' ability to name colors of affective words accounting for the ability to read words and name colors in the control conditions.

Working Memory.

Digit Sequencing Task (5 minutes). Subjects are presented auditorily with clusters of numbers (e.g., 936) of increasing length. They are asked to tell the experimenter the numbers in order, from lowest to highest. Measures: number of correct responses.

Continuous Performance Test (CPT). The CPT is a widely used measure of sustained attention, which is a preferred tool for assessing various mental functions. The CPT is a vigilance task requiring the monitoring of rapid information processing and the detection of briefly presented target stimuli. A higher processing load version of the CPT has been proven useful for measuring visual information processing and attentive capacity. Subject responses were recorded automatically on a diskette using the CPT machine (Sunrise Systems V2.20, Pembroke, Massachusetts). Numbers between 0 and 9 were randomly presented. The target stimulus was the number 9 preceded by the number 1. Each subject undertook two CPT sections: the unmasked task and masked task. During the unmasked session subjects responded to the target stimulus by pressing a button.

According to signal detection theory, the fundamental task of this test was to discriminate between the signal (target) and noise (non-target). This computerized test takes about 10 minutes to administer.

Trail Making A+B. The test is administered in two parts, A and B. The subject must first draw lines to connect consecutively numbered circles on one sheet (Part A) and then connect the same number of consecutively numbered and lettered circles on another worksheet by alternating between the two sequences, (Part B). Time: 10 minutes.

Wide Range Achievement Test (WRAT). Purpose: Designed to measure reading recognition, spelling, and arithmetic computation. Score: 3 scores: Spelling, Arithmetic, and Reading subtests (Reading is the only subtest administered to subjects herein; about 5 minute duration at screening only). This subtest requires subjects to recognize and name letters and pronounce words out of context. Time: 5 minutes for the Reading subtest. Authors: Joseph F. Jastak and Sarah Jastak. Publisher: Jastak Associates, Inc., Wilmington, Del.

Clinician Administered PTSD Scale (CAPS). The CAPS is a structured interview for assessing core and associated symptoms of PTSD. It assesses the frequency and intensity of each symptom using standard prompt questions and explicit, behaviorally-anchored rating scales. The CAPS yields both continuous and dichotomous scores for current and lifetime PTSD symptoms. Intended for use by experienced clinicians, it also can be administered by appropriately trained paraprofessionals. Data from a large-scale psychometric study of the CAPS have provided impressive evidence of its reliability and validity as a PTSD interview.

Connor-Davidson Resilience Scale. Made up of 25 items, each rated on a 5-point scale (0-4), with higher scores reflecting greater resilience. Resilience is a measure of stress coping ability and an important index in vulnerability to anxiety, depression, and stress reactions. Self-administered; Time: 5 minutes.

PTSD Check List-Stressor-Specific Version (PCL-S). This is a self-administered rating scale assessing PTSD symptoms. Time: 10 minutes.

BDI-II. A widely used instrument for detecting depression, and clinically very sensitive. 21 items to assess the intensity of depression in clinical and normal subjects (range 0-63). Each item is a list of four statements arranged in increasing severity about a particular symptom of depression. Self-administered; Time: 10 minutes.

Heinrich-Carpenter Quality of Life. Scale used to rate a number of measures associated with quality of life issues. Time: 10 minutes.

CGI (Clinical Global Impression scale). This is a commonly used, 3-item psychiatric scale to assess the clinician's overall general clinical assessment of improvement on a scale from 1-7. Clinicians rate the following three items: the Severity of Illness; Global Improvement; and Efficacy Index. Item 1 is rated on a 7-point scale (1 =normal to 7=extremely ill); item 2 on a seven point scale (1 =very much improved to 7=much worse); and item 3 on a four point scale (1 =none to 4 =outweighs therapeutic). Clinician time: 5 minutes.

Hillside Adverse Events (AE) Scale. The Hillside AE Scale is a form designed to rate five categories of symptoms on “Intensity” and “Relationship” to study. The categories include the following: behavioral, neurological cardiovascular, autonomic, and other. The five point “intensity” scale ranges from 0=not applicable, 1=not present to 4=severe. The five point “relationship” scale ranges from 1=none to 5=definite. This scale is administered weekly (at both scheduled Study Visits 1-6 and telephone check-in contact). In this assessment, subjects are asked a series of questions regarding possible side effects, to rate their severity (0-5) and the likelihood that the side effects are related to the study medication (0-4). All answers regarding side effects that are scored as a 2, 3, or 4 are reviewed and clinically addressed by the study physicians on the “intensity” scale. Time: 10 minutes.

Planned Analysis. The proposed pilot study is a randomized, placebo-controlled, double-blind, adjunctive 8-week treatment of a stable outsubject SSRI regimen (treatment-as-usual), preceded by a 2-week placebo-only lead-in period (total study duration 10 weeks); 30 subjects are randomized to either placebo (n=15) or PG augmentation (n=15). Before data analysis, major demographic variables, treatment, and clinical outcomes in the subjects that completed the neurocognitive and psychiatric rating scale testing compared to those who did not are examined. All neurocognitive and psychiatric variables are examined in terms of their distribution properties to determine whether baseline differences in neurocognitive or psychiatric rating scale scores existed between treatment groups. Analysis of treatment effects uses the mixed-models approach to repeated-measures analysis of variance (SAS Institute, Cary, N.C.) with baseline and endpoint scores as dependent variables, time as a within-subject repeated measure, and treatment group (PG or placebo) as a between-subjects fixed factor.

The primary efficacy measures in this study are subjects' scores on the BAC-A (the primary outcome measure for cognitive symptoms) and the CAPS (the primary outcome measure for PTSD symptoms). The primary analyses for this study are based on an intention-to-treat principle that includes in the analyses all randomized subjects. Secondary outcome measures for cognitive symptoms are the CPT and the Trails A and B. Other secondary outcome measures include PTSD symptoms as measured by the PCL, depressive symptoms as measured by the BDI-II, and overall functioning as measured by the Connor-Davidson Resilience Scale and the Heinrich-Carpenter Quality of Life questionnaire. For the BAC-A and CAPS primary outcome measures, the alpha is set at 0.05. For the secondary outcome measures, the critical alpha level is set at 0.01 for this pilot investigation.

### Study Outline

Subjects participating in the study go through the following steps:

1. Referred subjects meet with a member of the research team to discuss the study and the risks and benefits of participating. In addition, subjects are screened for exclusion and inclusion criteria (see above). If subjects are interested, the informed consent document is discussed with them. Subjects have the option of taking the informed consent with them and discussing the matter with family, friends, or clinicians. The subject's clinician is consulted about the subject's capacity to consent for the study and the appropriateness of the subject's enrollment in the study.

2. Once the informed consent is signed and accepted (an approximately 30 minute process), the subject proceeds to the initial screening procedures listed above in the Schedule of Events (Table 5). A psychiatric diagnosis (MINI: 15-minutes) is given by an investigator to confirm the subject's primary diagnosis.

3. Whether subjects are in good health is determined. A research physician examines subjects before starting the study. The physical exam, vital signs, medical and psychiatric history last about 25 minutes. Subjects have a venous puncture for laboratory tests listed in the Schedule of Events (Table 5). Since there is one report of palpitations in the scientific literature following PG administration, subjects receive an EKG at Visit 1. Subjects receive follow-up EKGs at Visit 4 and Visit 6 to make certain there are no changes in EKG tracings. Subjects with significant abnormal physical exam, blood tests, or EKG are excluded from the study and referred to their primary physician. Subjects then participate in cognitive testing (WRAT reading subtest) to determine cognitive functioning at intake.

4. Once subjects have successfully completed the screening process, they begin a two-week placebo-only lead-in period. On the days that subjects are given the study medication, a set of vital signs is taken including heart rate, blood pressure, respiratory rate, temperature, and weight. With the study medication, the subjects are told that they could be getting placebo (a sugar pill) or active compound (PG). The subjects are told that they might receive a different dose every two weeks. They are asked to take the medication twice a day; once in the morning and once at night. The initial dose in the first two weeks is placebo (2-week placebo lead-in phase).

5. After a week of placebo, a member of the research team contacts the subject to ask about compliance and to answer any questions. An investigator continues to contact subjects by phone every two weeks to ask about compliance and possible adverse events. These telephone check-in contacts are staggered with subject visits every two weeks as set forth in Table 5.

6. Subjects return every two weeks. Vital signs are repeated at each visit. A member of the research team asks subjects about side effects and adverse reactions. A venous puncture for blood is done to determine the serum level of PG and PG metabolites at each visit. Subjects are asked to return capsules of PG or placebo to determine compliance. The subject is then given a new supply of capsules (either PG or placebo, depending upon random assignment) every two weeks. At screening (Visit 1), and the final visit (Visit 6), subjects receive a TSH, prolactin, lipid panel, EKG, urine drug screen, and urinalysis; female subjects also receive a pregnancy test at screening (Visit 1), Visit 4, and at the final visit (Visit 6). A Chem 7, GI panel, and CBC are performed at each study visit (Visits 1 through 6). At Visit 4 (Week 6, or halfway through the placebo vs. PG phase), subjects receive an interim EKG, urine drug screen, and lipid panel.

7. At each visit, additional blood is collected (three extra red-top tubes) to be used to determine PG levels and PG metabolite levels. Serum is also used to characterize proteins and small molecules. At the end of these analyses, any remaining serum is destroyed.

8. At the end of week ten, the study medication stops. Subjects return for symptom measures, vital signs, and assessment of side effects. In addition, the physical exam is repeated.

9. The BAC-A (primary outcome measure; all others secondary outcome measures), BDI-II, the Heinrich-Carpenter Quality of Life scale, are performed at Visits 2, 4, and 6. The CGI is performed at Visits 2-6.

10. Drugs with a narrow therapeutic index (e.g., thioridazine, mesoridazine, ziprasidone, clozapine, etc.) are excluded; subjects taking these agents are not eligible for this study.

11. Adverse events are ascertained using the Hillside Adverse Events Form (copy attached). This scale is administered weekly (at both scheduled Study Visits 2-6 and telephone check-in contact) and requires 10 minutes to administer. In this assessment, subjects are asked a series of questions regarding possible side effects, to rate their severity (0-5), and the likelihood that the side effects are related to the study medication (0-4). All answers regarding side effects that are scored as a 2, 3, or 4 are reviewed and clinically addressed by the study physicians (1=not present, 2=mild, 3=moderate, 4=severe).

### Example 11

### Neuroactive Steroids Analysis in Veterans Returning from Iraq and Afghanistan

Neuroactive steroids (NS) modulate the stress response, increase following SSRIs, and can play a role in depression and PTSD. Whether NS are related to psychiatric symptoms in Operation Enduring Freedom (OEF) and Operation Iraqi Freedom (OIF) veterans was investigated.

NS serum levels in 90 male OEF/OIF veterans were determined by gas chromatography/mass spectrometry or radioimmunoassay. Psychiatric assessments included the Beck Depression Inventory (BDI-II), Davidson Trauma Scale (DTS), and Symptom Checklist-90-R(SCL-90-R). Canonical correlation analysis to determine if a linear relationship exists between predictor variables (NS, smoking, alcohol use, age, history of traumatic brain injury) and response variables (BDI-II, DTS, SCL-90-R) was statistically significant. Stepwise regression analysis was subsequently conducted.

ALLO levels were inversely associated with BDI-I1 scores (p=0.046) and PG levels were inversely associated with the SCL-90-R Global Severity Index (GSI; p=0.0491) in stepwise regression analysis. ALLO levels were inversely associated with SCL-90-R depression (p=0.018) and anxiety (p=0.048) subscales. DHEA was inversely associated with DTS re-experiencing symptoms (p=0.028). TBI was positively associated with DTS avoidance/numbing symptoms (p=0.042) and DTS total (trend p=0.070). Smoking was positively associated with the BDI-II, DTS total, and SCL-90-R GSI (p≦0.010).

Thus, it appears that NS were related to psychiatric symptoms in OEF/OIF veterans in this pilot investigation. ALLO findings were potentially consistent with antidepressant and anxiolytic actions of this NS. PG and DHEA thus represent candidate modulators of psychiatric symptoms. TB1 might be relevant to PTSD symptom severity. Smoking was associated with psychiatric symptoms, highlighting the importance of controlling for this variable.

### Example 12

### Neuroactive Steroids and Self-Reported Pain

To date, more than 1.4 million people have served during OEF/OIF. Deployments involve high levels of combat stress and many soldiers serve multiple tours of duty. Greater than one fourth of these returning veterans receive mental health or psychosocial diagnoses (Seal et al., 2007). An even greater proportion (nearly half) of these returning veterans report continued pain (Gironda et al., 2006). As a higher percentage of wounded soldiers survive than ever before (Gawande, 2004; Hoge et al., 2004; Hoge et al., 2006), reduction and alleviation of pain in this cohort is an acute need. Amelioration of these chronic pain symptoms via supplementation or modulation of endogenous compounds may represent a promising treatment strategy, and NS are potential candidates for this indication.

Several investigations report the analgesic effects of ALLO and other neuroactive steroids (NS) in animal models, but few data are currently available investigating the potential analgesic properties of NS in clinical populations.

The NS ALLO positively modulates inhibitory γ-aminobutyric acid type A (GABA_{A}) receptors and demonstrates pronounced analgesic and anxiolytic effects in animal models, yet studies examining the relationship between pain and ALLO in humans are very limited.

Based upon the analgesic actions observed for multiple NS in animals, levels of endogenous ALLO and other NS were correlated with pain perception in humans. The objective was to investigate whether there were any relationships between neuroactive steroid levels and four measures of self-reported pain in this OEF/OIF veteran population.

Neuroactive steroid serum levels in 90 male Operation Enduring Freedom/Operation Iraqi Freedom (OEF/OIF) veterans (mean age 39.3 years; 10.34 s.d.) were determined by gas chromatography/mass spectrometry or radioimmunoassay. Stepwise linear regression analyses were employed to determine relationships between self-reported pain measures and NS levels, controlling for age, current smoking status (24% smokers; 76% non-smokers), alcohol use (Alcohol Use Disorders Identification Test (AUDIT) score 6.2±6.64), and history of traumatic brain injury (13% positive with loss of consciousness by self-report; 87% negative). SAS default mode p<0.15 was used for terms entering into stepwise regression models in this exploratory analysis (SAS Institute Inc., Cary, N.C.; 1989).

Subjects were asked to rate their present pain levels in four categories from the Symptom Checklist-90-R: chest pain, low back pain, muscle soreness, and headache items. Each pain scale included five possible responses: no pain at all (level 0), a little pain (level 1), moderate pain (level 2), quite a bit of pain (level 3), and extreme pain (level 4).

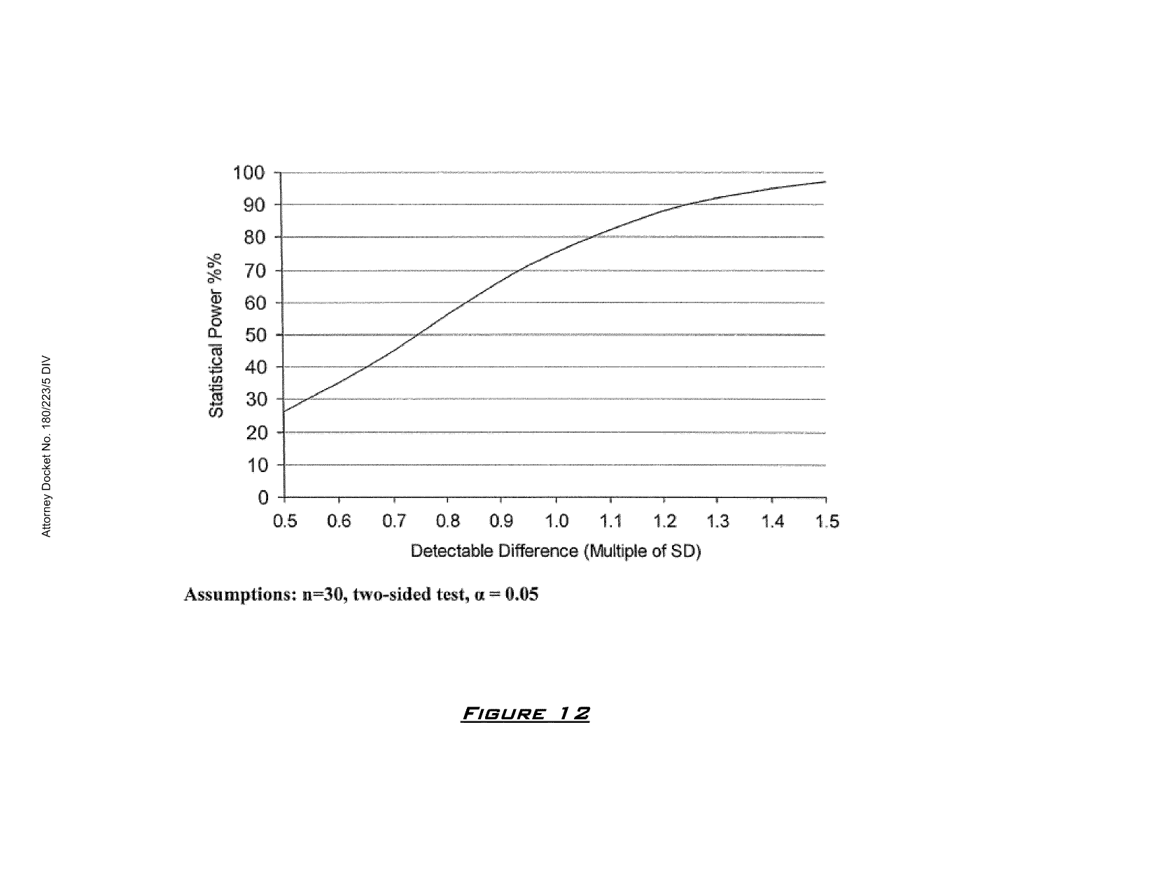

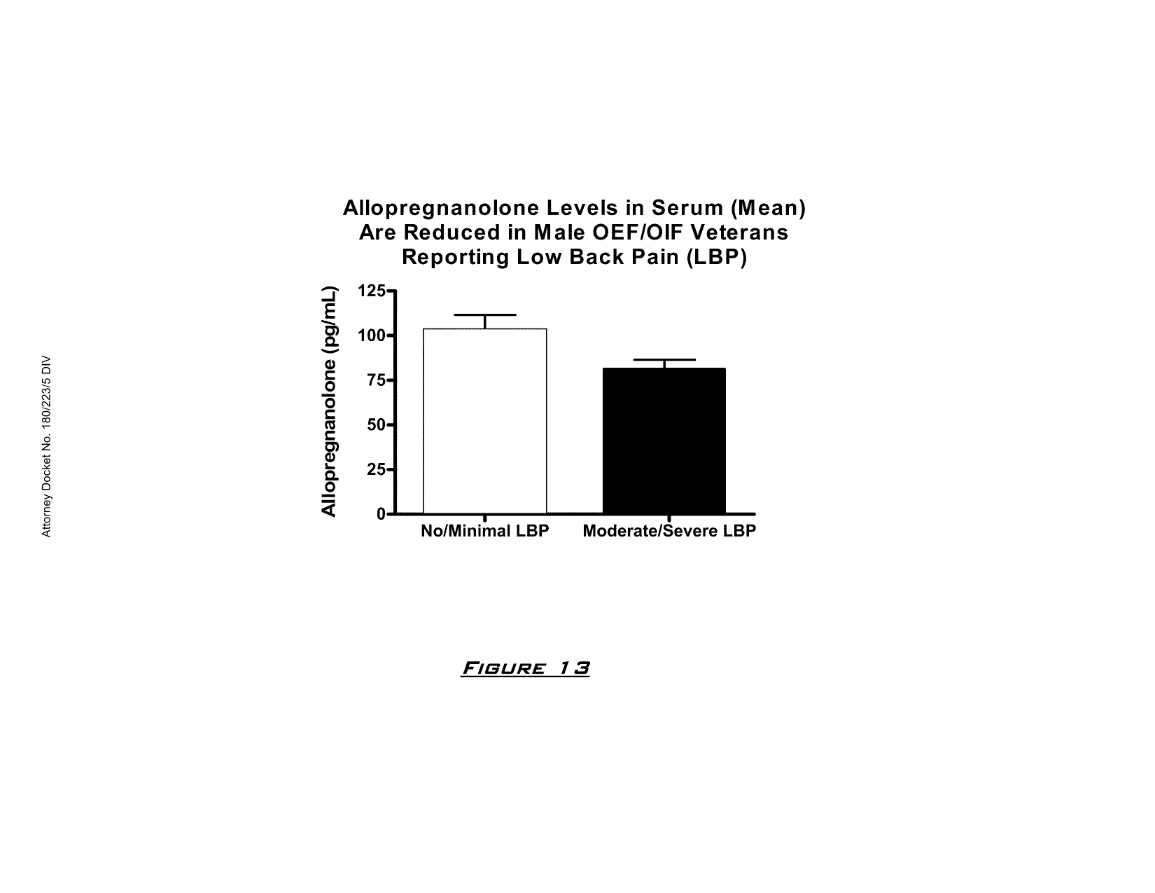

For data compilation and statistical comparison, responses of no/minimal pain (levels 0/1) were grouped, and responses of moderate to severe pain (levels 2/3/4) were grouped. The data are summarized in Table 6 and in FIGS. 13 and 14.

TABLE 6
Stepwise Regression Models

| Psychiatric | | | | | |
|---|---|---|---|---|---|
| Rating | | β | | | |
| Scale (Location | β | Coeff. p | Model p | | Summary of Key |
| of Pain) | Coeff. | value | value | R² | Findings |
| SCL-1 | | | 0.0734 | 0.04 | |
| (Headache) | | | | | |
| Alcohol Use | 0.0306 | 0.0734 | | | |
| SCL-12 (Chest) | | | <0.0028 | 0.16 | |
| ALLO | −0.0054 | 0.0134 | | | inverse association |
| | | | | | (p = 0.0134) |
| PG | −0.0003 | 0.1109 | | | |
| Progesterone | 0.0024 | 0.0012 | | | positive association |
| | | | | | (p = 0.0012) |
| SCL-27 | | | 0.0435 | 0.05 | |
| (Lower Back) | | | | | |
| ALLO | −0.0064 | 0.0435 | | | inverse association |
| | | | | | (p = 0.0435) |
| SCL-42 | | | 0.0021 | 0.17 | |
| (Muscle) | | | | | |
| DHEA | −0.0630 | 0.0238 | | | inverse association |
| | | | | | (p = 0.0238) |
| Progesterone | 0.8624 | 0.0987 | | | positive trend |
| | | | | | (p = 0.0987) |
| TBI | 1.2284 | 0.0017 | | | positive association |
| | | | | | (p = 0.0017) |

ALLO levels were higher in subjects reporting a 0 or 1 for chest pain severity (no/little pain) on the SCL-90 than in those reporting a 2, 3, or 4 for chest pain severity (moderate, severe, or extreme pain; mean levels 97.4±6.2 standard error of the mean (SEM) pg/ml vs. 67.9±6.4 SEM pg/ml, respectively; p=0.013, n=66 for no/little pain group and n=16 for moderate-to-severe pain group). ALLO levels were higher in subjects reporting a 0 or 1 for low back pain severity (no/little pain) than in those reporting a 2, 3, or 4 for chest pain severity (moderate to severe pain; mean levels 101.7±8.7 SEM pg/ml vs. 82.1±5.8 SEM pg/ml, respectively; p=0.044, n=42 for no/little pain group and n=40 for moderate-to severe pain group). DHEA levels were higher in subjects reporting a 0 or 1 for muscle soreness severity (no/little pain) than in those reporting a 2, 3, or 4 for chest pain severity (moderate to severe pain; mean levels 10.7±0.0 SEM ng/ml vs. 8.8±0.8 SEM ng/ml, respectively; p=0.024, n=54 for no/little pain group and n=28 for moderate-to-severe pain group).

ALLO levels in serum were inversely associated with chest pain (p=0.013) and low back pain (p=0.044). DHEA levels were inversely associated with muscle soreness (p=0.024). Traumatic brain injury was positively associated with muscle soreness (p=0.002).

ALLO findings were potentially consistent with the antinociceptive actions of this neuroactive steroid. DHEA and TBI might also be relevant to self-reported pain symptoms in OEF/OIF veterans. Neuroactive steroids thus represent possible therapeutic targets for pain and stress disorders.

DHEAS levels were also found to be positively associated with chest pain, suggesting that sulfonation may potentially impact the analgesic properties of NS.

Age, smoking, and alcohol use were not correlated with any reported pain measures.

### Example 13

### Neuroactive Steroids and Psychiatric Symptoms in OEF/OIF Veterans

Converging preclinical and clinical evidence suggest that neuroactive steroids (NS) play a role in depression and PTSD. The GABAergic NS ALLO demonstrates neuroprotective actions, modulates the stress response, enhances neurogenesis, and increases following selective serotonin reuptake inhibitors and certain antipsychotics. Reductions in ALLO have also been correlated with depression (Uzunov et al., 1998) and PTSD symptoms (Rasmusson et al., 2006). Thus, whether serum NS profiles were associated with depression and PTSD symptoms in veterans who served during Operation Enduring Freedom (OEF) and Operation Iraqi Freedom (OIF) was investigated.

NS serum profiles were determined by gas chromatography/mass spectrometry or radioimmunoassay in the same 90 male OEF/OIF male veterans discussed hereinabove in Example 12. Statistical methods employed included a canonical correlation to determine if there are statistically significant relationships between NS and psychiatric symptoms (p=0.018); stepwise linear regression analyses were performed to determine relationships between NS and psychiatric symptoms, controlling for smoking status, alcohol use, age, and h/o TBI; non-parametric analyses (Mann-Whitney U test statistic) were performed to compare the ratio of ALLO to its progesterone precursor, and the ratio of DHEA to its sulfated derivative DHEAS in depression and PTSD.

Various psychiatric rating scales were employed. As Primary Outcome Measures, Beck Depression Inventory-II (BDI-II) Total, Symptom Check List-90-R(SCL-90-R), Global Severity Index (GSI), and Davidson Trauma Scale (DTS) Total were employed. As Secondary Outcome Measures, DTS B (re-experiencing), C (avoidance/numbing), and D (hyperarousal) subscales, and SCL-90-R symptom constructs: Anxiety, Depression, Paranoid Ideation, and Psychoticism were employed. The results are summarized in Tables 7-9.

TABLE 7
Stepwise Regression Models - Primary Outcome Measures

| Psychiatric | | β | | | |
|---|---|---|---|---|---|
| Rating | β | Coeff. p | Model p | | Summary of |
| Scale | Coeff. | value | value | R² | Key Findings |
| BDI-II | | | 0.0002 | 0.25 | |
| ALLO | −0.0417 | 0.0459 | | | inverse association |
| | | | | | (p = 0.0459) |
| DHEAS | 0.0165 | 0.0277 | | | positive association |
| | | | | | (p = 0.0277) |
| Progesterone | 5.4832 | 0.1419 | | | |
| Smoking Status | 7.1449 | 0.0017 | | | positive association |
| | | | | | (p = 0.0017) |
| DTS Total | | | <0.0001 | 0.26 | |
| Progesterone | 29.7947 | 0.0094 | | | positive association |
| | | | | | (p = 0.0094) |
| TBI | 18.2148 | 0.0696 | | | positive trend |
| | | | | | (p = 0.0696) |
| Smoking Status | 0.0696 | 0.0001 | | | positive association |
| | | | | | (p = 0.0001) |
| SCL - Global | | | 0.0004 | 0.25 | |
| Severity Index | | | | | |
| ALLO | −0.0029 | 0.0543 | | | inverse trend |
| | | | | | (p = 0.0543) |
| PG | −0.0004 | 0.0494 | | | inverse association |
| | | | | | (p = 0.0494) |
| DHEAS | 0.0008 | 0.1319 | | | |
| Progesterone | 0.9847 | 0.0034 | | | positive association |
| | | | | | (p = 0.0034) |
| Smoking Status | 0.4091 | 0.0099 | | | positive association |
| | | | | | (p = 0.0099) |

TABLE 8
Stepwise Regression Models - Secondary Outcome Measures

| Psychiatric | | β | | | |
|---|---|---|---|---|---|
| Rating | β | Coeff. p | Model p | | Summary of Key |
| Scale | Coeff. | value | value | R² | Findings |
| DTS B | | | <0.0001 | 0.28 | |
| Subscale (re- | | | | | |
| experiencing) | | | | | |
| DHEA | −0.4871 | 0.0275 | | | inverse association |
| | | | | | (p = 0.0275) |
| Progesterone | 15.7911 | 0.0002 | | | positive association |
| | | | | | (p = 0.0002) |
| TBI | 5.1572 | 0.0876 | | | positive trend |
| | | | | | (p = 0.0876) |
| Smoking Status | 7.4691 | 0.0025 | | | positive association |
| | | | | | (p = 0.0025) |
| DTS C | | | <0.0001 | 0.26 | |
| Subscale | | | | | |
| (avoidance/ | | | | | |
| numbing) | | | | | |
| Progesterone | 12.4001 | 0.0173 | | | positive association |
| | | | | | (p = 0.0173) |
| TBI | 14.8139 | 0.0415 | | | positive association |
| | | | | | (p = 0.0415) |
| Smoking | 0.0173 | <0.0001 | | | positive association |
| | | | | | (p < 0.0001) |
| DTS D | | | 0.0025 | 0.14 | |
| Subscale | | | | | |
| (hyperarousal) | | | | | |
| DHEAS | 0.0137 | 0.0850 | | | positive trend |
| | | | | | (p = 0.0850) |
| Smoking Status | 8.7534 | 0.0027 | | | positive association |
| | | | | | (p = 0.0027) |
| SCL - Anxiety | | | 0.0004 | 0.25 | |
| ALLO | −0.0034 | 0.0477 | | | inverse association |
| | | | | | (p = 0.0477) |
| DHEA | −0.0417 | 0.0178 | | | inverse association |
| | | | | | (p = 0.0178) |
| DHEAS | 0.0011 | 0.0788 | | | positive association |
| | | | | | (p = 0.0788) |
| Progesterone | 0.7471 | 0.0266 | | | positive association |
| | | | | | (p = 0.0266) |
| SCL - | | | 0.0008 | 0.22 | |
| Depression | | | | | |
| ALLO | −0.0038 | 0.0232 | | | inverse association |
| | | | | | (p = 0.0232) |
| PG | −0.0003 | 0.1329 | | | |
| Progesterone | 1.2638 | 0.0006 | | | positive association |
| | | | | | (p = 0.0006) |
| Smoking Status | 0.3546 | 0.0506 | | | positive trend |
| | | | | | (p = 0.0506) |
| SCL - Paranoid | | | <0.0001 | 0.36 | |
| Ideation | | | | | |
| PG | −0.0005 | 0.0335 | | | inverse association |
| | | | | | (p = 0.0335) |
| DHEAS | 0.0009 | 0.1245 | | | |
| Progesterone | 1.0746 | 0.0082 | | | positive association |
| | | | | | (p = 0.0082) |
| Alcohol Use | 0.0218 | 0.1355 | | | |
| Smoking Status | 0.6318 | 0.0036 | | | positive association |
| | | | | | (p = 0.0036) |
| SCL - | | | 0.0004 | 0.25 | |
| Psychoticism | | | | | |
| ALLO | −0.0020 | 0.1261 | | | |
| PG | −0.0003 | 0.0524 | | | inverse trend |
| | | | | | (p = 0.0524) |
| DHEAS | 0.0011 | 0.0187 | | | positive association |
| | | | | | (p = 0.0187) |
| Progesterone | 0.7189 | 0.0171 | | | positive association |
| | | | | | (p = 0.0171) |
| Smoking Status | 0.3705 | 0.0098 | | | positive association |
| | | | | | (p = 0.0098) |

TABLE 9
Neuroactive Steroid Levels

| | | Mann-Whitney |
|---|---|---|
| Psychiatric Rating Scale | NS Ratio | Test p Value |
| DTS (<10 vs. ≧40) | ALLO/PROG | 0.0449* |
| | DHEA/DHEAS | 0.0704 |
| BDI-II (<10 vs. ≧20) | ALLO/PROG | 0.0089* |
| | DHEA/DHEAS | 0.0316* |

*p ≦ 0.05;
**p ≦ 0.01 (two tailed)

ALLO levels were inversely associated with BDI-II scores (p=0.046) and PG levels are inversely associated with the SCL-90-R Global Severity Index (GSI; p=0.049) in stepwise regression analysis. ALLO levels were inversely associated with SCL-90-R depression (p=0.018) and anxiety (p=0.048) subscales. DHEA was inversely associated with DTS re-experiencing symptoms (p=0.028). Smoking was positively associated with the BDI-II, DTS total, and SCL-90-R GSI (p≦0.010). ALLO/Progesterone and DHEA/DHEAS ratios were reduced in veterans with depression (p=0.0089 and p=0.0449, respectively) or PTSD (p=0.0316 and p=0.0704, respectively). These findings are summarized in Table 10.

TABLE 10
Summary of Findings

| Neuroactive | |
|---|---|
| Steroid | Summary of Findings |
| ALLO | Lower levels associated with: |
| | ↑ Depressive Symptoms (BDI-II, SCL-Depression) |
| | ↑ Symptoms of Anxiety (SCL-Anxiety) |
| PG | Lower levels associated with: |
| | ↑ Global severity of symptoms (SCL-GSI) |
| | ↑ Paranoid Ideation (SCL-Paranoia) |
| DHEA | Lower levels associated with: |
| | ↑ Symptoms of PTSD (DTS-B) |
| | ↑ Symptoms of Anxiety (SCL-Anxiety) |
| DHEAS | Higher levels associated with: |
| | ↑ Depressive symptoms (BDI-II) |
| | ↑ Psychoticism (SCL-Psychoticism) |
| Progesterone | Higher levels associated with: |
| | ↑ Symptoms of PTSD (DTS-Total, DTS-B, DTS-C) |
| | ↑ Global severity of symptoms (SCL-GSI) |
| | ↑ Depressive symptoms (SCL-Depression) |
| | ↑ Symptoms of Anxiety (SCL-Anxiety) |
| | ↑ Paranoid Ideation, Psychoticism (SCL-P, SCL-PSY) |

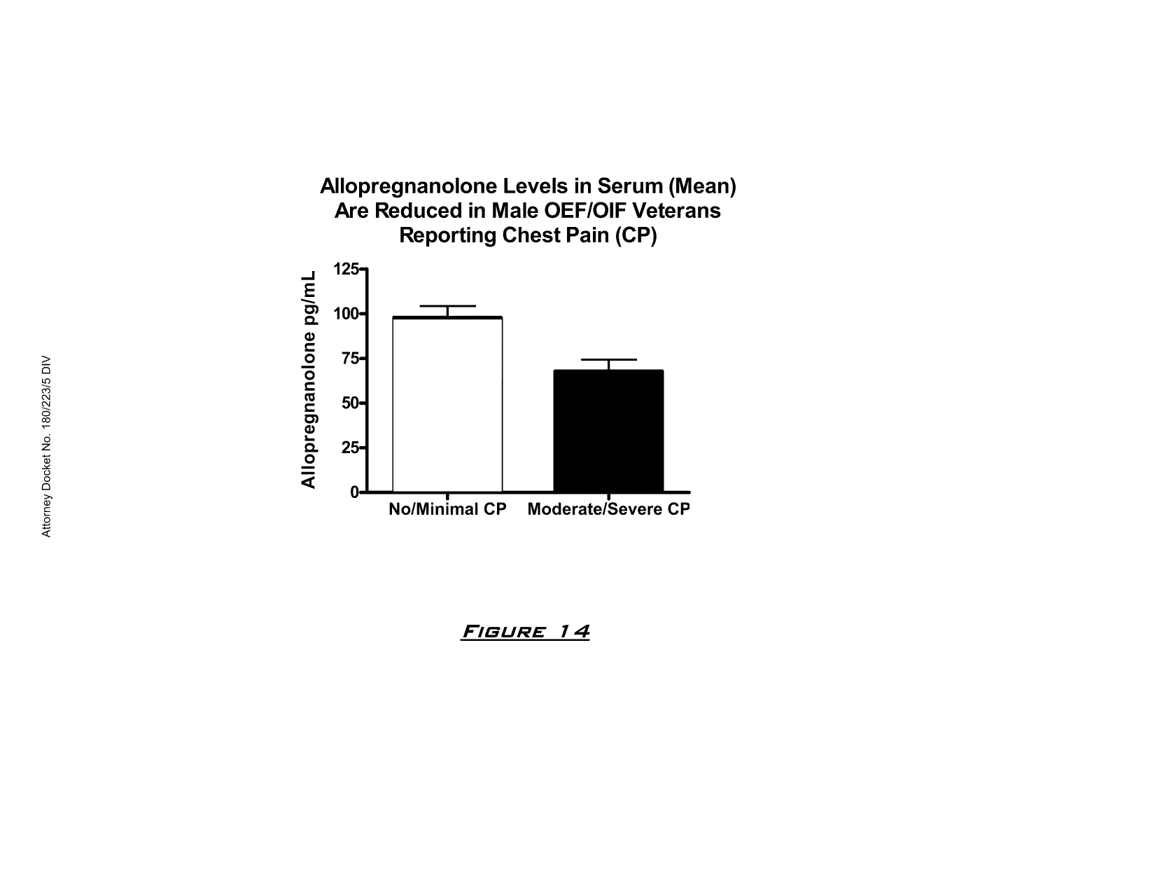

Additionally, median ALLO/PG ratios were found to be decreased as PTSD symptoms increased (increasing DTS Score; see FIG. 15) and also as depression symptoms increased (BDI-II Scale; see FIG. 16).

Neuroactive steroids were related to psychiatric symptoms in OEF/OIF veterans. ALLO findings were consistent with its antidepressant and anxiolytic actions. Smoking was associated with psychiatric symptoms. ALLO/Progesterone and DHEA/DHEAS ratios were reduced in veterans with depression or PTSD, highlighting the importance of examining NS metabolic pathways. TBI was associated with more severe PTSD symptomatology, and interestingly, smoking was associated with psychiatric symptoms, underlying the necessity of controlling for this variable.

### Materials and Methods for Examples 14 and 15

Lithium and Valproate Administration. Male Wistar Kyoto rats (purchased from Harlan Sprague Dawley Inc., Indianapolis, Ind.) were treated chronically with lithium or valproate for four weeks at doses achieving therapeutic and physiologically relevant levels (0.74±0.31 mEq/I for lithium, measured by atomic absorption; 41.6±3.2 μg/ml for valproate, measured by immunoassay), and compared to saline vehicle administration for four weeks (n=9 per condition). Animals were fed with control rodent chow or chow containing a low dose of lithium (1.2 g Li₂CO₃/kg) or valproate (10 g/kg) for one week to allow the animals to slowly acclimate to the drug, and were then fed with chow containing a full dose of lithium (2.4 g Li₂CO₃/kg) or valproate (20 g/kg) for three weeks. On the last day of the treatment, animals were sacrificed, and trunk blood was collected to measure lithium and valproate levels in the serum. Brain tissues were dissected on ice and frozen in dry ice immediately. Frontal cortex was defined as the cortical projection area of the mediodorsal thalamic nucleus. Animal use procedures were in accordance with the Guide for the Care and Use of Laboratory Animals of the National Institutes of Health.

Bcl-2 Knockout (KO) Mice. Founder mice were purchased from The Jackson Laboratory (Bar Harbor, Me.). Adult Bcl-2 KO mice were crossbred with wild type outbred mice to generate heterozygote mice. Bcl-2 heterozygote siblings were then interbred to generate homozygotes (−/−), heterozygotes (−/+), and wild type (+/+) littermates. The genotypes of the offspring were determined by PCR analysis. Mice tail biopsy genomic DNA was isolated using DNeasy Tissue kit (Qiagen, Valencia, Calif.). The Neo primers had the following sequences: 5′-CTTGGGTGGAGAGGCTATTC-3′ (SEQ ID NO: 1) and 5′-AGGTGAGATGACAGGAGATC-3′ (SEQ ID NO: 2), and yielded a 280 basepair (bp) fragment. The BcI-2 primers had the following sequences: 5′-CTTIGTGGAACTGTACGGCCCCAGCATGCG-3′ (SEQ ID NO: 3) and 5′-ACAGCCTGCAGCTTTGTTTCATGGTACATC-3′ (SEQ ID NO: 4), and yielded a 215 by fragment. Wild type animals would have only the Bcl-2 fragment, heterozygote animals would have both fragments, and Bcl^{-/-} animals would have only the Neo fragment.

The heterozygous offspring appeared entirely normal and were fertile. Bcl-2^{-/+} mice and their wild type littermates were employed because most of the null (−/−) offspring died prior to weaning.

8-week old mice (weight about 20-30 grams) were sacrificed by cervical dislocation without anesthetization, and brain tissues were dissected on ice and frozen in dry ice immediately. Western blots performed and reported previously (Einat et al., 2005) showed lower Bcl-2 protein levels in heterozygotes than in wild type littermates. Animal use procedures were in accordance with the Guide for the Care and Use of Laboratory Animals of the NIH. Strain Name: B6129S2-Bcl^{2tm1Sjk}/J; Jackson Labs Stock Number: 002265; Wild type: B6129SF2/J; Jackson Labs Stock Number: 101045.

NS Analyses: Gas Chromatography/Mass Spectrometry (GC/MS), preceded by High Performance Liquid Chromatography (HPLC). NS analyses in rat and mouse frontal cortex were performed as described in Marx et al., 2006a; Marx et al., 2006b; and Marx et al., 2006c. Rodent brain tissue was homogenized in 5 volumes of distilled water containing about 2200 cpm (4400 dpm/injection) of tritiated NS (Perkin Elmer, Waltham, Massachusetts) to detect the HPLC fraction containing the NS of interest. Deuterated ALLO (D4-ALLO, 400 pg) and deuterated PG (D4-PG, 400 pg) were used as internal standards. Supernatants were extracted three times with three volumes of ethyl acetate and dried under nitrogen prior to HPLC purification. Samples were derivatized utilizing heptafluorobutyric acid anhydride, injected onto an Agilent 5973 Mass Spectrometer (MS) coupled to an Agilent 6890N Gas Chromatograph (GC; Agilent Technologies, Inc., Santa Clara, Calif.), and analyzed in the negative ion chemical ionization mode (NICI) utilizing methane as the reaction gas and helium as the carrier gas. Mass spectrometer single ion monitoring (SIM) mode was utilized to focus on the most abundant ion fragment for each steroid derivative. Only peaks with a signal-to-noise ratio greater than or equal to 5:1 were integrated. The limit of NS detection with this method was 2 pg for ALLO and 10 pg for PG. Mean intra-assay coefficients of variation were 3.9% for ALLO and 3.0% for PG.

Statistical Analysis. For rat experiments utilizing chronic lithium and valproate administration, data were analyzed by ANOVA with post-hoc Dunnett tests. Data from Bcl-2 knockout mouse experiments were analyzed by unpaired t-tests.

### Example 14

### Neuroactive Steroids in Chronic Lithium Treatment

Many neuroactive steroids (NS) demonstrate neurotrophic and neuroprotective actions, including protection against apoptosis via Bcl-2 protein. NS are altered in postmortem brain tissue from subjects with bipolar disorder, and several agents with efficacy in mania elevate NS in rodents. It was thus hypothesized that lithium and valproate might elevate NS, and compensatory NS increases might occur in Bcl-2 knockout mice.

To that end, NS levels (ALLO and PG) were determined in frontal cortex by negative ion chemical ionization gas chromatography/mass spectrometry in male Wistar Kyoto rats treated chronically with lithium, valproate, or vehicle. NS were also investigated in heterozygous Bcl-2 knockout mice.

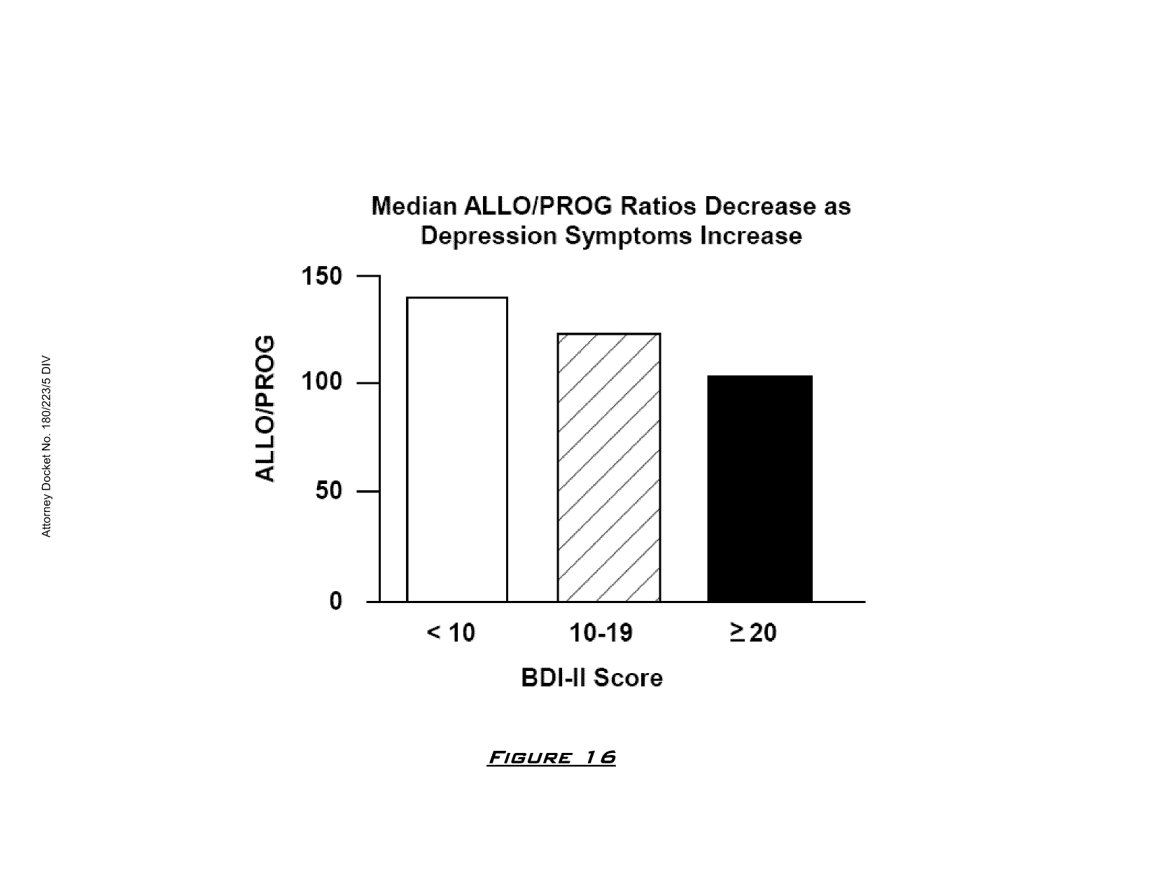

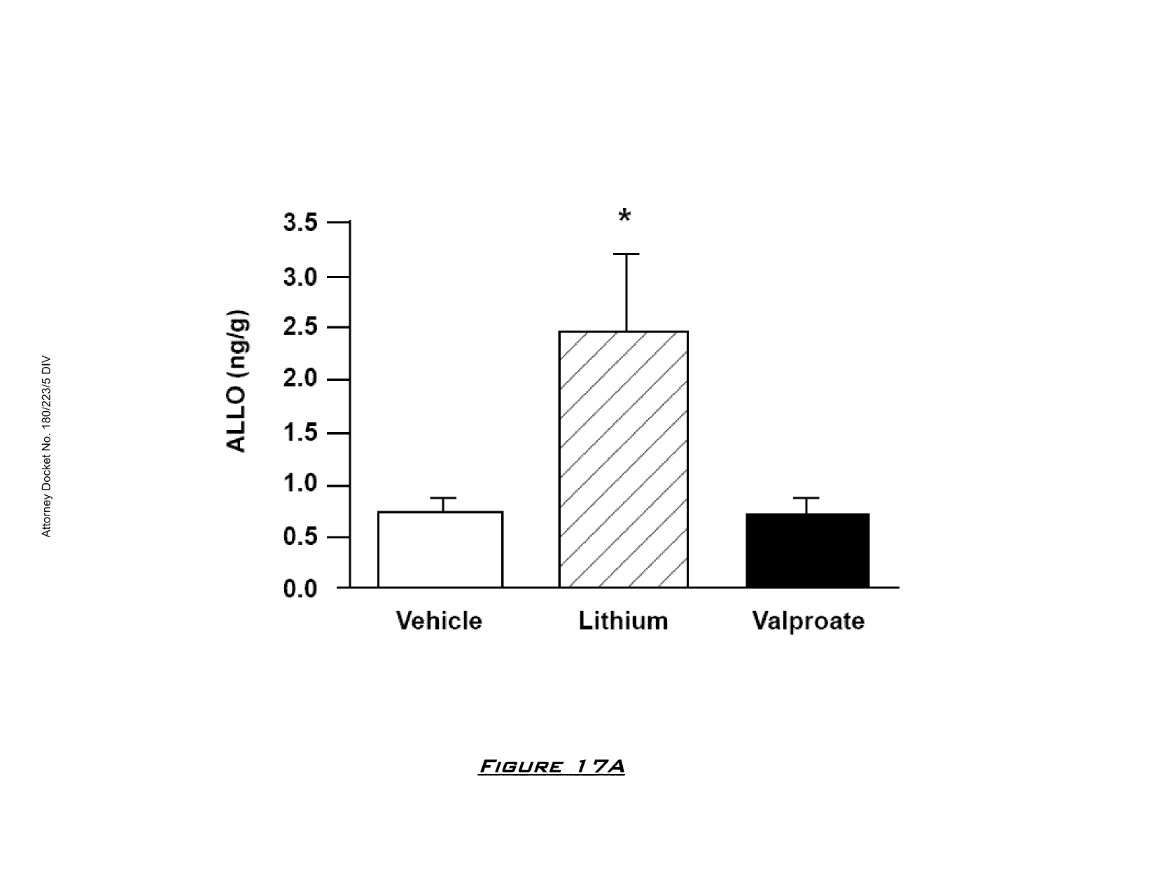

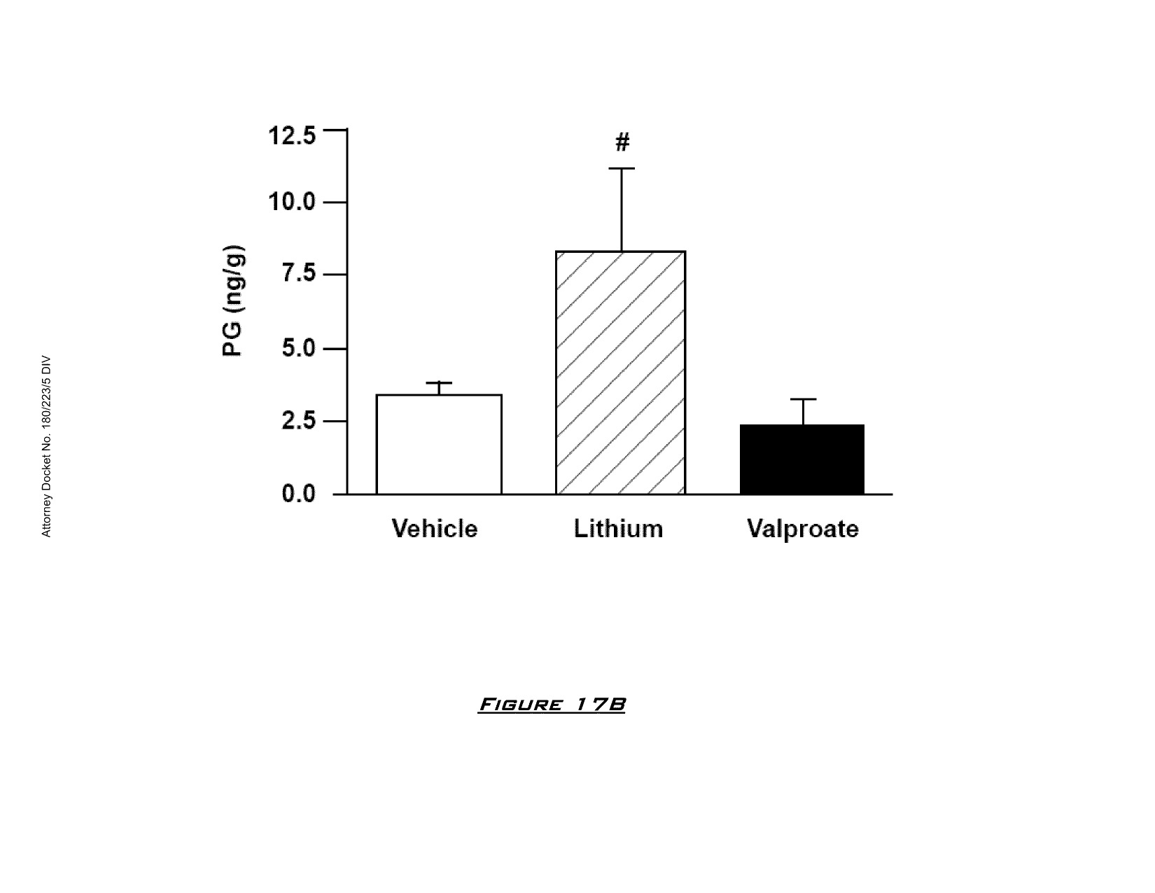

ALLO levels were significantly increased in rat frontal cortex following chronic lithium administration compared to vehicle (see FIG. 17A; mean levels 2.42 ng/g±0.75 SEM vs. 0.71 ng/g±0.11 SEM, respectively; ANOVA p=0.017, F=4.85, df 2,24, post-hoc Dunnett p<0.05, n=9 per condition). PG levels also tended to be higher in rat frontal cortex following chronic lithium administration compared to vehicle (see FIG. 17B; mean levels 8.19 ng/g±2.95 SEM vs. 3.37 ng/g±0.50 SEM, respectively; ANOVA p=0.069, F=3.00, df 2,24, post-hoc Dunnett p=0.09, n=9 per condition). In contrast, chronic valproate administration alters neither ALLO nor PG levels (see FIGS. 17A and 17B).

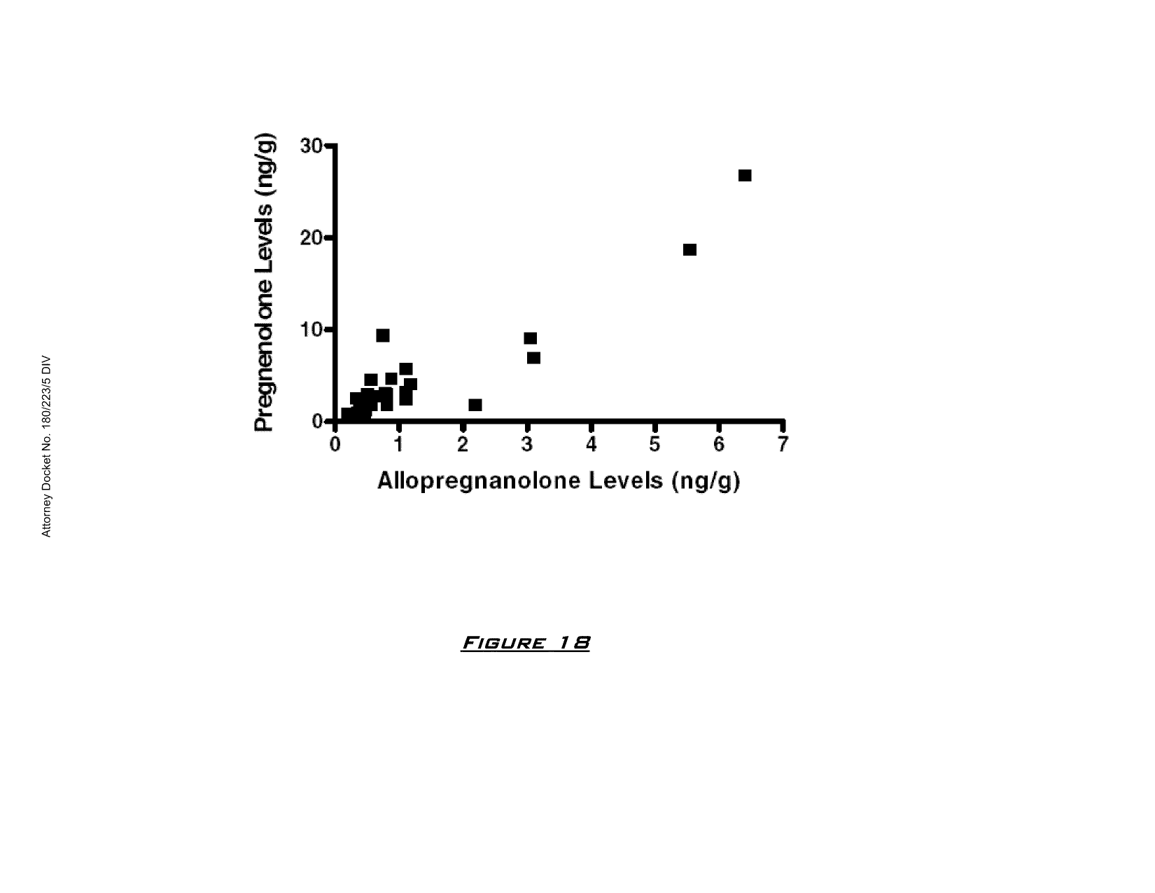

Since PG can be metabolized to ALLO, the correlation between the levels of these two NS were examined in rat frontal cortex. It was determined that ALLO levels were positively correlated with PG levels (see FIG. 18; Pearson correlation coefficient r=0.92, p<0.0001, n=27 XY pairs), with high degrees of correlation throughout this set of subjects, at low, medium, and high levels of these neuroactive steroids.

### Example 15

### Neuroactive Steroids in Bcl-2 Knockout Mice

Based on previous reports indicating that ALLO protects against apoptosis (Charalampopoulos et al., 2004; Xilouri & Papazafiri, 2006), it was hypothesized that compensatory upregulation of ALLO levels might occur in Bcl-2 knockout mice compared to wild type mice. However, no differences in frontal cortex ALLO levels between these two groups were observed (3.96 ng/g±0.33 SEM in Bcl-2 knockout mice vs. 4.02 ng/g±0.31 SEM in wild type control mice, unpaired t-test p>0.05, n=10 per condition).

PG levels, in contrast, were significantly higher in heterozygous Bcl-2 knockout mice compared to wild type mice (mean levels 4.03 ng/g±0.78 SEM vs. 1.65 ng/g±0.23 SEM, respectively, unpaired t-test p=0.009, n=10 per condition). In these mice, PG and ALLO levels were not significantly correlated.

### Example 16

### Neuroactive Steroids in Traumatic Brain Injury (TBI)

Rodent data suggest that ALLO is elevated following alcohol administration and is relevant to its behavioral effects. Also, PG can be metabolized to progesterone. In accordance with the presently disclosed subject matter progesterone metabolism to ALLO is a mechanistic component of neuroprotective effects in traumatic brain injury (TBI). Based on data disclosed herein, PG administration constitutes a precursor loading strategy resulting in elevated ALLO levels, and increases in ALLO levels can be clinically therapeutic in traumatic brain injury.

### Example 17

### Neuroactive Steroids in Alcohol Use Disorders

In a rodent model, the PG metabolite ALLO is elevated following alcohol administration and is relevant to its behavioral effects. Since the presently disclosed data suggest that PG administration results in 5-fold increases in downstream ALLO metabolite formation, PG and/or ALLO administration constitutes a potential target in the treatment of alcohol use disorders.

### Materials and Methods for Examples 18-21

Subjects. The experiments disclosed in EXAMPLE 18 were approved by the Duke University Medical Center Institutional Review Board, and written informed consent was obtained from all subjects prior to their participation. The subjects included 28 male smokers between the ages of 18 and 65 recruited from the community by newspaper and radio advertisements and by word-of-mouth for a smoking cessation protocol. All participants smoked at least 15 cigarettes per day of a brand having an FTC-rated nicotine yield of at least 0.5 mg. Subjects with serious medical conditions by history or physical exam were excluded (see Table 11 for subject characteristics). Subjects with a psychiatric disorder other than nicotine dependence (DSM-IV criteria) or who reported current smokeless tobacco use were also excluded.

TABLE 11
Subject Characteristics of Male Smokers

| Baseline Characteristics | Mean | n | SD |
|---|---|---|---|
| Age | 41.4 | 28 | 14.1 |
| Number of cigarettes per day | 22.4 | 27 | 6.5 |
| FTND score | 5.0 | 25 | 2.0 |
| Age when smoking started | 19.2 | 26 | 5.0 |
| Nicotine levels (ng/ml) in saliva | 911.9 | 23 | 843.7 |
| Cotinine levels (ng/ml) in saliva | 282.8 | 22 | 144.6 |

All subjects underwent a screening session and received multiple standardized questionnaires, including surveys of smoking habits and four measures of craving severity. Measures included the Fagerstrom Test for Nicotine Dependence (FTND; Fagerstrom, 1978), the addiction subscale of the Ikard Smoking Motivation Questionnaire (ISMQ; Ikard et al., 1969), the craving item on the Reasons to Smoke (RTS) questionnaire (1-7 scale), and the negative affect and craving subscales of the Shiffman-Jarvik Withdrawal Questionnaire (Shiffman & Jarvik, 1976). All samples of saliva for determination of cotinine and nicotine levels and of blood for steroid analyses were obtained at the screening interview prior to randomization to specific smoking cessation treatment arms; the majority of blood and saliva samples were obtained between 10:00 A.M. and 2:00 P.M. (23 subjects). Sample procurement times were unavailable for five subjects.

Radioimmunoassays. Serum DHEAS, ANDRO, free testosterone, progesterone, and estradiol levels were determined by commercially available kits according to manufacturer directions (Diagnostic Systems Laboratories, Los Angeles, Calif.).

Gas Chromatography/Mass Spectrometry Preceded by HPLC. GC/MS preceded by HPLC was performed essentially as set forth hereinabove. Serum (1.0 ml) was homogenized in five volumes of distilled water containing 4,000 dpm of tritiated neuroactive steroid (Perkin Elmer) to detect the HPLC fraction containing the neuroactive steroid of interest, as well as a constant amount of deuterated ALLO and deuterated PG as the internal standards. Supernatants were extracted three times with three volumes of ethyl acetate. HPLC purification was performed on a 1100 Series Agilent HPLC equipped with a Packard 500TR Flow Scintillation Analyzer for radiopeak detection. Each steroid was collected into a separate fraction based upon the retention time of its radioactive analogue, utilizing hexane, tetrahydrofuran, and ethanol as the mobile phase and a Phenomenex LiChrosorb DIOL (5 μm particle size) 250 4.6 mm column. The standards and samples were then derivatized utilizing heptafluorobutyric acid anhydride (HFBA) and injected onto an Agilent 5973 mass spectrometer (MS) coupled to an Agilent 6890 N gas chromatograph (GC) equipped with an Agilent HP-5MS 30 m 0.250 mm 0.25 μm capillary column. They were analyzed in the negative ion chemical ionization mode (N ICI) utilizing methane as the reaction gas and helium as the carrier gas. The derivatized steroids of interest subjected to NICI yield negative ions in a mass range between m/z 100 and 700. In addition to the GC retention time characteristic of each steroid, the structural identification of each neuroactive steroid assayed was provided by its unique mass fragmentation pattern. Mass spectrometer single ion monitoring (SIM) mode was utilized to focus on the most abundant ion fragment for each HFBA steroid derivative (ALLO 474.4 and 494.3; PG 492.3 and 472.4). For neuroactive steroid quantification, the standard curve for the steroid of interest was prepared by combining varied known quantities of steroid (Steraloids) ranging from 2 to 3,000 pg with a constant amount of the respective deuterated internal standard. Identical to the samples, the standard curve was extracted three times in ethyl acetate prior to HPLC purification and GC/MS injection; standard curve r²=0.99 for each neuroactive steroid. The area under the peak of each known quantity of neuroactive steroid was divided by the area under the peak of the internal standard. This ratio was plotted on the y-axis against known quantities of each steroid to generate the standard curve. Only peaks with a signal to noise ratio greater or equal to 5:1 were integrated. The limit of neuroactive steroid detection was 2 pg for ALLO and 10 pg for PG.

GC/MS: salivary nicotine and cotinine determination. Nicotine and cotinine determinations were performed as previously described (Jacob et al., 1981; Rose et al., 2003). It has been demonstrated that salivary cotinine levels are highly correlated with serum cotinine levels (Jarvis et al., 2003).

Statistical analyses determining potential correlations between steroid levels and nicotine dependence severity measures, negative affect, and salivary nicotine and cotinine levels. Because DHEAS levels were negatively correlated with age as expected (r=−0.612, p=0.0005), partial correlations controlling for age were performed with raw DHEAS values to determine potential associations between DHEAS and the above variables (nicotine dependence severity measures, negative affect rating scale, and salivary nicotine and cotinine levels), SAS Version 8. ANDRO (r=−0.590, p=0.0012) and free testosterone (r=−571, p=0.0015) levels were also inversely related with age, and therefore partial correlations controlling for age were also performed for these steroid analyses. ALLO, PG, progesterone, and estradiol levels were not correlated with age in this cohort (p>0.05 for each steroid) and therefore analyses were not adjusted for age and Pearson correlation coefficients were determined in the analyses of these steroids (Prism 4.03).

Based on serum availability, ALLO, PG, DHEAS, progesterone, free testosterone, and estradiol serum levels were determined in all 28 subjects, and serum ANDRO levels were determined in 27 subjects (ANDRO level was unavailable for one subject secondary to inadequate serum volume). Missing data included a small number of subjects with unavailable FTND (n=3), ISMQ (n=1), RTS questionnaire (n=1), or Shiffman-Jarvik Withdrawal Questionnaire (n=3) scores. Five subjects did not provide saliva samples for nicotine and cotinine analyses. One outlying cotinine level was 3.72 SD above the mean and was omitted, as per statistical consultation. These missing data points were addressed via case-wise deletion.

Log transformation was considered for some variables when appropriate. In this small sample set, however, it was difficult to establish the presence or absence of normal distributions with certainty. Other than the study dataset, there was little information available to suggest that study variables assumed non-normal distributions or to justify particular transformations. It was therefore assumed that these variables were likely distributed normally (as is the case with many direct measures of biological analytes) and untransformed neuroactive steroid levels were reported in concordance with prior investigations (Uzunova et al., 1998; Strous et al., 2003; Schmidt et al., 2005).

Since multiple hypotheses were tested in the same dataset, a more conservative significance threshold was adopted, p≦0.01. Analyses yielding p values≦0.01 were designated as statistically significant, analyses yielding p values≦0.05 (bu t>0.01) were designated as marginally significant, and p values≦0.10 were described as possible trends.

Statistical analyses, steroid correlation matrix. Pearson correlation coefficients were determined for all steroid levels. These are presented in the steroid correlation matrix (see Table 14). The p values≦0.01 were designated as statistically significant, as described above.

### Example 18

### Neuroactive Steroids and Male Smokers

### Subject Characteristics

Subject characteristics, and mean salivary nicotine and cotinine levels (±standard deviation) are presented in Table 11 above.

### Example 19

### Serum Neuroactive Steroid Levels in Male Smokers

Mean serum steroid levels (±standard deviation) in this cohort of male smokers (n=27-28) are presented in Table 12.

TABLE 12
Serum Steroid Levels in Male Smokers

| Steroid | Mean | n | SD |
|---|---|---|---|
| DHEAS (μg/dl) | 195.0 | 28 | 117.2 |
| ALLO (pg/ml) | 203.2 | 28 | 133.4 |
| PG (pg/ml) | 639.4 | 28 | 402.2 |
| Progesterone (ng/ml) | 0.81 | 28 | 0.3 |
| ANDRO (ng/ml) | 3.6 | 27 | 1.2 |
| Free testosterone (pg/ml) | 10.9 | 28 | 3.6 |
| Estradiol (pg/ml) | 27.8 | 28 | 8.7 |

### Example 20

Neuroactive Steroids and Negative Affect, Nicotine Dependence Severity

### Measures, and Salivary Nicotine and Cotinine Levels

DHEAS levels were inversely correlated with a number of measures, and these DHEAS results are summarized in Table 13.

TABLE 13
Correlations Between Serum DHEAS Levels, and Negative
Affect and Nicotine Dependence Severity Measures
(with Partial Correlations Controlling for Age)

| Ratings Measure | Mean | p | n |
|---|---|---|---|
| Negative affect (Shiffman-Jarvik Withdrawal | −0.60 | 0.002** | 25 |
| Questionnaire subscale) | | | |
| Reasons to Smoke questionnaire craving | −0.43 | 0.030* | 27 |
| item | | | |
| Fagerstrom Test for Nicotine Dependence | −0.38 | 0.067 | 25 |
| Ikard Smoking Motivation questionnaire | −0.38 | 0.059 | 27 |
| addiction subscale | | | |
| Craving subscale (Shiffman-Jarvik | −0.33 | 0.120 | 25 |
| Withdrawal questionnaire) | | | |

*p ≦ 0.05 marginally significant;
**p ≦ 0.01 statistically significant.

The DHEAS levels were inversely correlated with the negative affect subscale of the Shiffman-Jarvik Withdrawal Questionnaire (r=−0.60, p=0.002, n=25; statistically significant) and the craving item of the Reasons to Smoke (RTS) Questionnaire (r=−0.43, p=0.03, n=27; marginally significant), with partial correlations controlling for age. Possible trends included the following: DHEAS levels tended to be negatively correlated with the Fagerstrom Test for Nicotine Dependence (FTND) scores (r=−0.38, p=0.067, n=25) and the Ikard Smoking Motivation Questionnaire (ISMQ, Ikard et al., 1969) addiction subscale (r=−0.38, p=0.059, n=27), with partial correlations controlling for age. No other steroids were correlated with measures of negative affect or nicotine dependence severity.

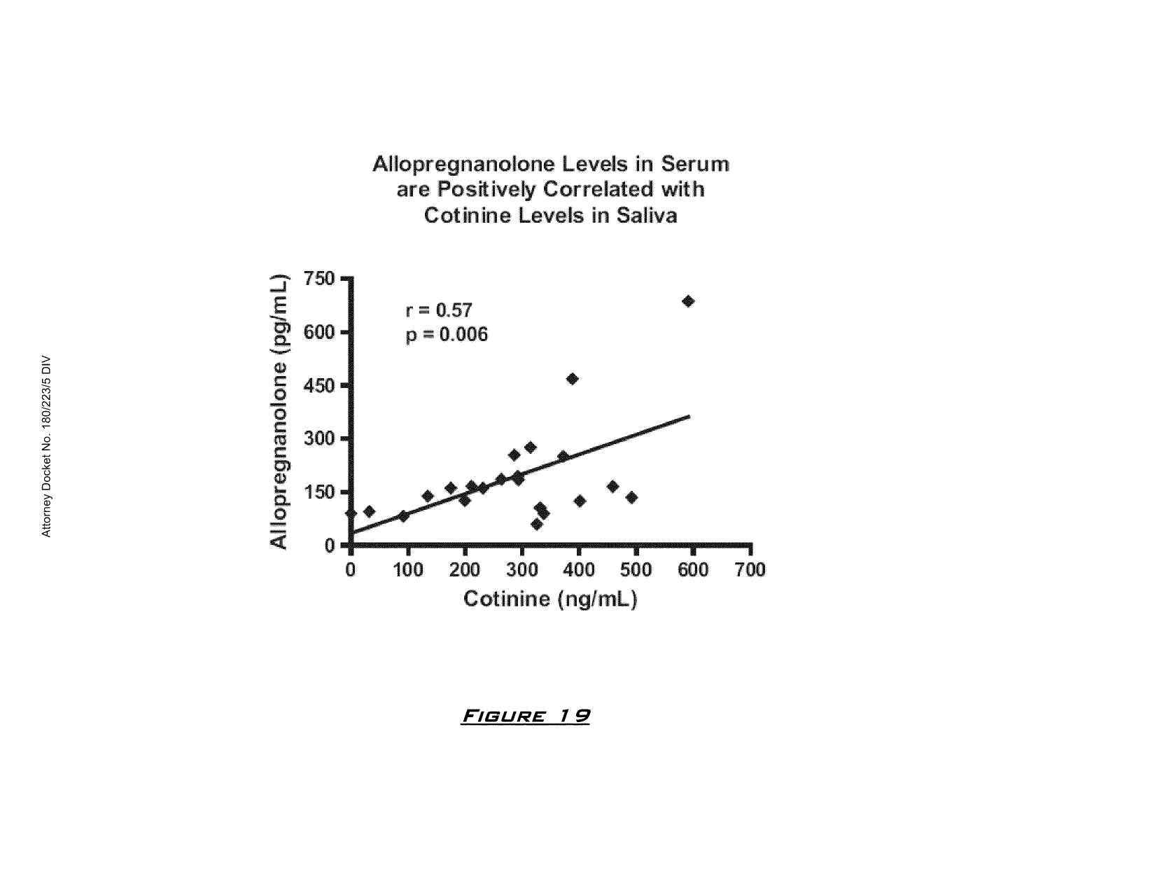

ALLO levels were positively correlated with salivary cotinine levels (r=0.57, p=0.006, n=22; statistically significant, see FIG. 19). Log transformation was considered and results were substantially unchanged (r=0.54, p=0.010, n=22; statistically significant).

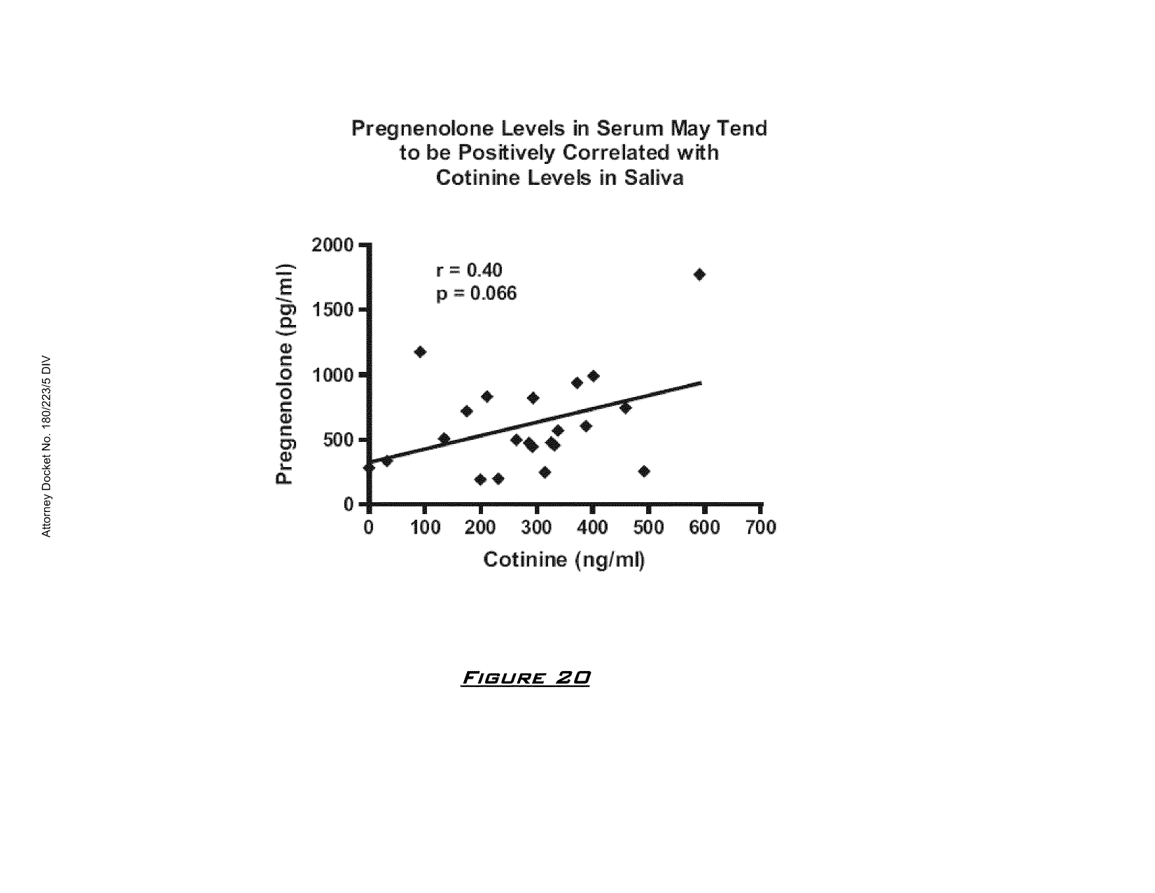

PG levels also tended to be positively correlated with cotinine levels (r=0.40, p=0.066, n=22; possible trend, see FIG. 20). Since one outlying data point might be highly influential in this analysis (potentially driving a possible trend), the analysis was repeated with the outlier removed and the p value was determined to indeed exceed 0.10 following this computation. No other steroids were correlated with cotinine levels. There were no significant or marginally significant correlations between any steroids tested and salivary nicotine levels.

### Example 21

### Steroid Correlation Matrix

Pearson correlation coefficients were determined among the steroids tested, and a steroid correlation matrix is presented in Table 14.

PG is a potential precursor to many steroids, and in this cohort of male smokers, serum pregnenolone levels were positively correlated with all steroids tested. Statistically significant Pearson correlation coefficients (p≦0.01) were demonstrated for the association between PG and the following steroids: ALLO (r=0.50, p=0.006, n=28), DHEAS (r=0.56, p=0.002, n=28), progesterone (r=0.87, p<0.001, n=28), and ANDRO (r=0.71, p<0.001, n=27). Marginally significant Pearson correlation coefficients (p≦0.05) were demonstrated for the association between PG and free testosterone (r=0.47, p=0.011, n=28) and the association between PG and estradiol (r=0.41, p=0.031, n=28). DHEAS levels were positively and significantly correlated with progesterone (r=0.51, p=0.006, n=28), ANDRO (r=0.70, p<0.001, n=27), and free testosterone (r=0.55, p=0.002, n=28). A number of additional statistically significant and marginally significant correlations were observed among other steroids (see Table 14 for details).

TABLE 14
Steroid Correlation Matrix

| | | | | | | Free | |
|---|---|---|---|---|---|---|---|
| | | | | Proges- | | Testos- | |
| | | ALLO | PG | terone | ANDRO | terone | Estradiol |
| DHEAS | r | 0.217 | 0.556** | 0.506** | 0.699** | 0.553** | 0.218 |
| | p | 0.268 | 0.002 | 0.006 | <0.001 | 0.002 | 0.266 |
| | n | 28 | 28 | 28 | 27 | 28 | 28 |
| ALLO | r | | 0.504** | 0.550** | 0.313 | 0.301 | 0.393* |
| | p | | 0.006 | 0.002 | 0.112 | 0.120 | 0.038 |
| | n | | 28 | 28 | 27 | 28 | 28 |
| PG | r | | | 0.872** | 0.707** | 0.474** | 0.407* |
| | p | | | <0.001 | <0.001 | 0.011 | 0.031 |
| | n | | | 28 | 27 | 28 | 28 |
| Progesterone | r | | | | 0.676** | 0.615** | 0.608** |
| | p | | | | <0.001 | <0.001 | 0.001 |
| | n | | | | 27 | 28 | 28 |
| Androstenedione | r | | | | | 0.770** | 0.312 |
| | p | | | | | <0.001 | 0.113 |
| | n | | | | | 27 | 27 |
| Free | r | | | | | | 0.394* |
| Testosterone | p | | | | | | 0.038 |
| | n | | | | | | 28 |

r: Pearson correlation coefficient;
p: p value;
n: number of subjects
*p ≦ 0.05 marginally significant;
**p ≦ 0.01 statistically significant

### Example 22

### Neuroactive Steroids in Tobacco Cessation

Neuroactive steroid DHEAS is inversely associated with negative affect and craving measures in male smokers. Treatment with DHEA has been associated with the alleviation of depressive symptoms (Schmidt et al., 2005; Strous et al., 2003; Wolkowitz et al., 1999). Negative affect frequently accompanies smoking. DHEA is a pharmacological target for smoking cessation. DHEA can thus potentially decrease craving for cigarettes and also attenuate negative affect during withdrawal, potentially reducing relapse likelihood via two distinct mechanisms.

In accordance with the presently disclosed subject matter, it has also been demonstrated that ALLO (a PG metabolite) is correlated with salivary cotinine levels, consistent with general hypothalamic-pituitary-adrenal axis (HPA axis) activation observed in smokers. PG also tended to be correlated with salivary cotinine levels. Reductions in both ALLO (Uzunov et al., 1998) and PG (George et al., 1994) have been associated with depressive symptoms. In accordance with the presently disclosed subject matter it is submitted that PG impacts negative affect in smokers, potentially via metabolism to ALLO. DHEA and PG therefore represent potential agents that can have utility in smoking cessation.

Elaborating, a pilot study is provided herein, which involves administering one-time DHEA (400 mg), PG (400 mg) or placebo to healthy male smokers following overnight abstinence from cigarettes. This intervention with these neuroactive steroids attenuates craving measures following overnight smoking cessation. Thus, neuroactive steroids can be candidate therapeutic molecules for smoking cessation.

### Discussion of the Examples

Neuroactive Steroids and Schizophrenia. The proof-of-concept randomized controlled trial utilizing PG in subjects with schizophrenia described in EXAMPLE 1 suggested that this neurosteroid could have therapeutic potential in this disorder, particularly for cognitive symptoms. Specifically, serum PG increases following augmentation with this neurosteroid are strongly correlated with two state-of-the-art cognitive assessment batteries (BACS and MATRICS). Subjects with the greatest increases in PG levels demonstrated the greatest improvements in cognitive symptoms, as assessed by BACS and MATRICS (r=0.79, p=0.02 and r=0.70, p=0.05, respectively).

The disclosed data thus support a role for PG in the treatment of schizophrenia. For example, it has been determined that the “gold standard” antipsychotic clozapine, a drug with proven superior efficacy in the treatment of refractory schizophrenia, markedly increases the neurosteroid PG in rat hippocampus, cerebral, cortex, and serum (Marx et al., 2006). These elevations might contribute to the superior therapeutic action of clozapine in this subject population. In addition, clozapine has recently been approved by the FDA for the treatment of suicidal behaviors, and it is possible that clozapine-induced elevations in PG can be relevant to these clinical actions and thus represents a new target for intervention.

Supporting this possibility, the data disclosed herein also demonstrate that PG was significantly reduced in parietal cortex in subjects with schizophrenia who died by suicide compared to subjects with schizophrenia who died of other causes. PG therefore represents a promising pharmacological target in subjects with schizophrenia and/or bipolar disorder for a number of symptom domains including cognitive impairment and suicidality.

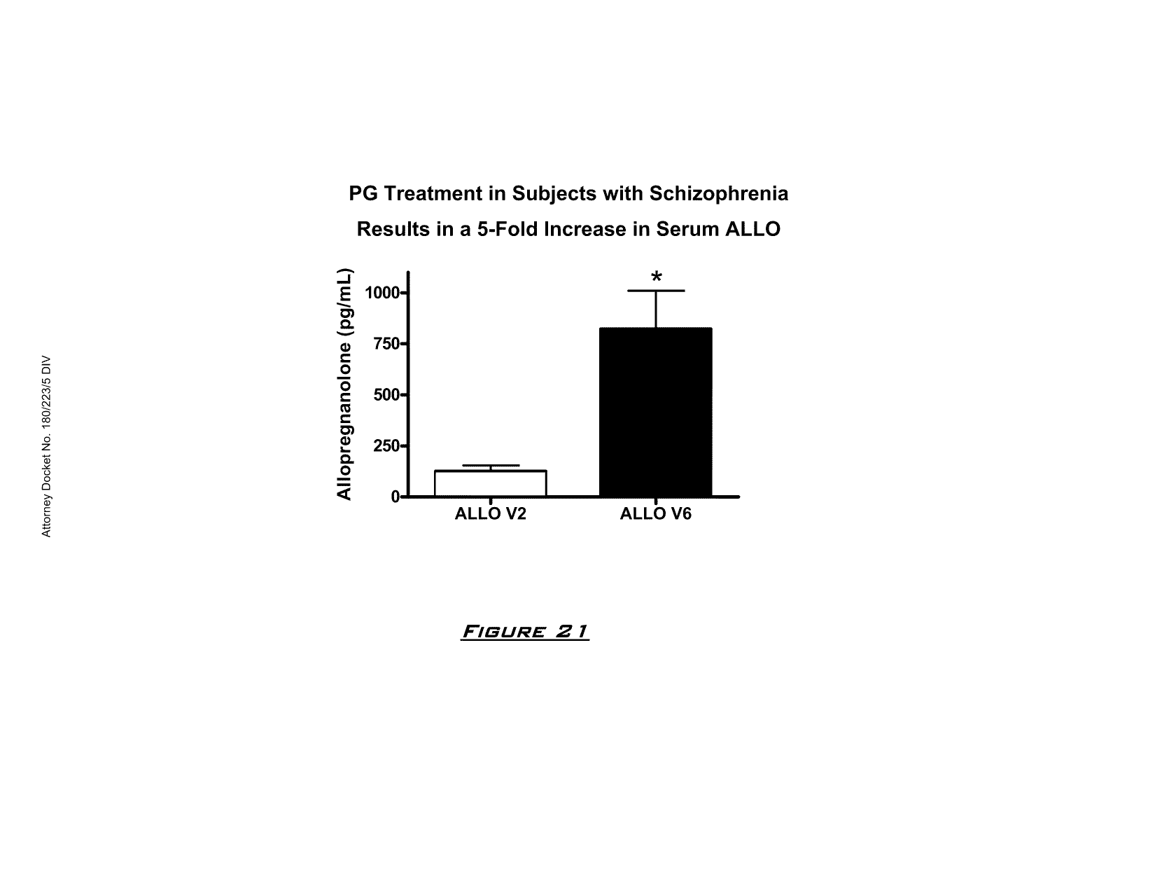

Furthermore, PG can be metabolized to the GABAergic neurosteroid ALLO, which is also elevated following clozapine administration in rodent models and might contribute to its unique therapeutic effects (Marx et al., 2003). The data disclosed herein also demonstrate that PG administration increased serum ALLO levels in schizophrenia subjects over 5-fold (see FIG. 21), and therefore represents a viable precursor loading strategy resulting in clinically therapeutic ALLO elevations. In patients with schizophrenia who were randomized to adjunctive pregnenolone administration, serum allopregnanolone levels as determined by mass spectrometry-based techniques increased over 5-fold following eight weeks of treatment with pregnenolone compared to baseline levels (see FIG. 21).

Neuroactive Steroids and Alzheimer's Disease. Neuroactive steroid levels are also disclosed herein to be altered in subjects with Alzheimer's disease (AD). For example, ALLO is significantly reduced in postmortem prefrontal cortex (PFC) brain tissue; and inversely correlated with neuropathological disease stage. Decreased ALLO levels in Alzheimer's disease could therefore have functional significance. Indeed, restoration of ALLO levels can be clinically efficacious in this disorder, and ALLO restoration can be accomplished with a PG precursor loading strategy. The data discussed herein above, demonstrating that PG administration in humans results in over 5-fold increases in ALLO are consistent with this approach.

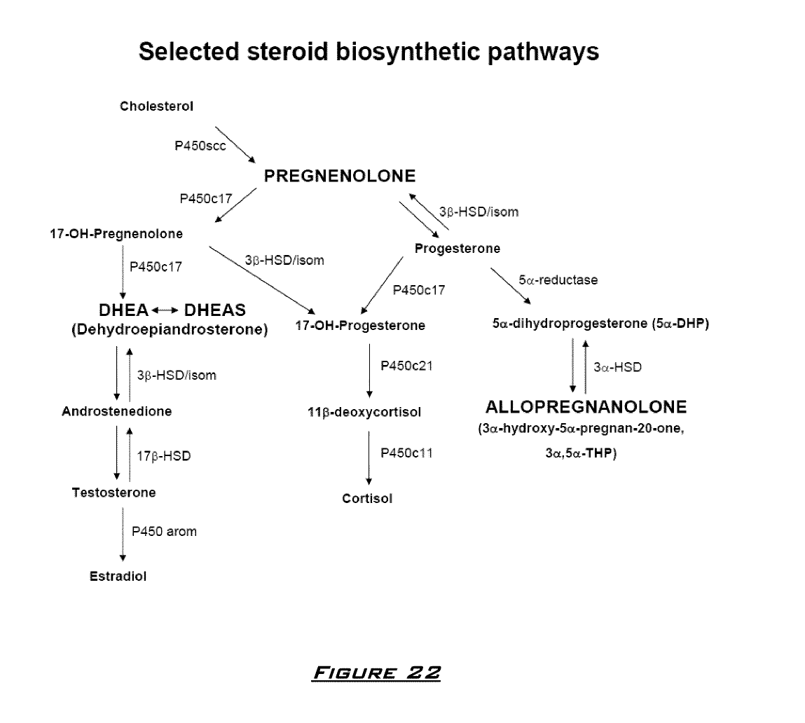

The mechanism(s) leading to reductions in PFC ALLO levels in AD remain to be elucidated. It is possible that neurosteroidogenesis might be disrupted in AD, but this would not necessarily seem to be a generalized effect. For example, DHEA levels in PFC were higher in subjects with AD (in contrast to reduced ALLO levels), suggesting that steroid synthesis capacity might be preserved (and possibly even enhanced) in certain circumstances. In Niemann-Pick type C mice (which demonstrate age-related reductions in brain ALLO levels), the expression and activity of the neurosteroidogenic enzyme 3α-hydroxysteroid dehydrogenase (3α-HSD) decrease markedly with age. It is therefore possible that the expression and activity of the 3α-HSD enzyme, which catalyzes the formation of ALLO from its precursor 5α-dihydroprogesterone (see FIG. 22), might also be reduced in AD.

In addition to reduced ALLO levels in two brain regions in subjects with Alzheimer's disease compared to cognitively intact control subjects, DHEA levels are altered (elevated) in both temporal and prefrontal cortex in subjects with this disorder. PG levels are also increased in temporal cortex (trend in prefrontal cortex). DHEA and PG are also elevated in cerebrospinal fluid in subjects with Alzheimer's disease compared to cognitively intact control subjects (p=0.03 DHEA, trend p=0.10 PG). Furthermore, levels in CSF are strongly correlated with DHEA and PG levels in temporal cortex in the same subject cohort (r=0.59 for DHEA, r=0.57 for PG; p<0.0001 for both analyses). CSF levels therefore appear to reflect central brain levels of these neurosteroids. Since both of these neurosteroids are elevated in temporal cortex in subjects with Alzheimer's disease, these CSF neurosteroids can thus potentially constitute a surrogate biomarker for Alzheimer's disease diagnosis and course.

Neuroactive Steroids and Bipolar Disorder. Neuroactive steroids might also be useful for treating subjects with Bipolar Disorder. Disclosed herein is the observation that chronic lithium administration produced significant increases in ALLO levels (as well as a trend toward increases in PG) in rat frontal cortex, while administration of valproate did not alter NS levels. Additionally, a strong correlation between the concentrations of PG and its metabolite ALLO was demonstrated in frontal cortex. ALLO concentrations were not altered in heterozygous Bcl-2 knockout mice, despite its reported role in protection against apoptosis; PG levels, however, were significantly increased in these animals. In this strain of mice, ALLO and PG levels were not correlated.

While chronic lithium administration, a primary treatment strategy for bipolar disorder, can result in neurotoxicity in subjects with identifiable clinical risk factors (nephrogenic diabetes insipidus, old age, abnormal thyroid function, impaired renal function; Oakley et al., 2001), two week administrations of lithium at clinically relevant doses have been shown to enhance neurogenesis in rat hippocampus, increasing both Bcl-2 levels and the percentage of new cells that display a neuronal phenotype (Chen et al., 2000; Chen et al., 1999).

Since ALLO dose-dependently increases proliferation of rat hippocampal neuroprogenitor cells and human cerebral cortical neural stem cells at physiologically relevant concentrations, and also increases expression of genes that promote progression through the cell cycle (Wang et al., 2005), it was hypothesized that lithium treatment might produce elevations in brain ALLO. Chronic lithium administration more than tripled ALLO levels in rat frontal cortex (see FIG. 17A), raising the possibility that lithium-induced elevations in ALLO might contribute to increased neurogenesis following lithium administration and potentially impact neuroplasticity.

Similar to lithium-induced ALLO increases disclosed herein, previous reports indicate that second generation antipsychotics that demonstrate efficacy in mania such as clozapine and olanzapine also elevate allopregnanolone levels in rodent brain (Barbaccia et al., 2001; Marx et al., 2003). Taken together, these data suggested that ALLO elevations might contribute to the therapeutic efficacy exhibited by lithium. It should be noted, however, that mood stabilizers such as lithium might actually be more versatile in their actions on NS levels, as a previous augmentation study demonstrates that these compounds reverse antidepressant-induced NS increases (Schule et al., 2007). Previous reports also indicate that ALLO has pronounced neuroprotective effects in a mouse model of Niemann-Pick type C disease (Griffin et al., 2004) and a rat model of traumatic brain injury (Djebaili et al., 2005), suggesting a potential role for ALLO induction in the neuroprotective effects of lithium as well (Gray et al., 2003).

While ALLO was not altered in frontal cortex of heterozygous BcI-2 knockout mice compared to ALLO levels in wild type mice, PG levels are significantly higher in these animals. It is possible that PG elevations in heterozygous Bcl-2 knockout mice reflected a compensatory mechanism that results in the normalization of downstream ALLO metabolite levels in this BcI-2 strain. Interestingly, although neuroprotective effects have also been attributed to valproate (Chuang, 2005), increases were observed in neither ALLO nor PG levels in response to chronic valproate administration. The discrepancies in the NS responses to these two mood stabilizers might represent another potential mechanistic difference in the neuroprotective properties of these two compounds (Hennion et al., 2002; Jin et al., 2005; Mora et al., 1999; Mora et al., 2002).

Finally, like the antipsychotic clozapine (Meltzer et al., 2003), lithium decreases suicidality (Baldessarini et al., 2006). Disclosed herein is the discovery that parietal cortex PG levels in patients with schizophrenia who died by suicide were significantly reduced in comparison to patients with schizophrenia who died of other causes (see also Bradford, 2006). In light of this association between reduced brain PG and increased suicidality, it is a logical possibility that increasing NS levels might contribute to the modulation of suicidal behaviors by lithium. Previous reports support this hypothesis, since clozapine elevates both PG (Marx et al., 2006a) and ALLO (Barbaccia et al., 2001; Marx et al., 2003) in rat brain, and prior evidence suggests links between reduced ALLO and depression (Uzunova et al., 2006).

In summary, the data disclosed herein suggested that elevations in ALLO following chronic lithium administration might be relevant to its therapeutic efficacy, as well its impact on neuroplasticity and neuroprotection. NS might also be implicated in Bcl-2 mechanisms relevant to lithium actions.

Neuroactive Steroids and Smoking Cessation. Disclosed herein is a pilot study investigating serum steroid levels in 28 male smokers at baseline. Several associations between neuroactive steroid levels and negative affect, nicotine dependence severity measures, and salivary cotinine levels were identified. Significant interrelationships between a number of steroids in male smokers were determined. These findings thus suggest potential relevance to the neurophysiology of nicotine dependence and the development of new agents for smoking cessation based on neuroactive steroids.

To elaborate, this study revealed that DHEAS levels were inversely correlated with negative affect, adjusting for age, as measured by the negative affect subscale of the Shiffman-Jarvik Withdrawal Questionnaire. This finding remained significant even when a more conservative p value≦0.01 was applied.

The relationships between smoking, negative affect, and stress have received considerable attention in recent years. It has been demonstrated that negative affect and stress are associated with relapse among smokers attempting cessation (Kassel et al., 2003; Sinha, 2005). Links between smoking and depression have also been demonstrated, and this relationship might be bidirectional; specifically, depressive symptoms might predispose an individual to smoking initiation, and smoking might subsequently increase risk for the development of depressive symptoms (Kassel et al., 2003; Quattrocki et al., 2000; Paperwalla et al., 2004). Smokers have higher rates of major depression compared to nonsmokers (Kendler et al., 1993; Glassman et al., 1990; Breslau et al., 1993). Finally, smoking cessation might precipitate depressive symptoms, and subjects with a history of major depression can be particularly vulnerable to a recurrence in depressive symptoms during smoking cessation (Quattrocki et al., 2000; Paperwalla et al., 2004). One prospective study demonstrated that subjects with a history of major depression enrolled in a smoking cessation trial who remained abstinent from smoking had a seven-fold greater risk of depression recurrence compared to subjects with a depression history who continued to smoke (Glassman et al., 2001).

The data presented herein demonstrating that DHEAS levels in male smokers were inversely correlated with negative affect might thus be relevant to the above investigations suggesting numerous links between smoking and depression. Specifically, DHEA administration decreases depressive symptoms in subjects with depression (Schmidt et al., 2005; Wolkowitz et al., 1999) and schizophrenia (Strous et al., 2003). The finding presented herein that higher serum DHEAS levels were associated with lower degrees of negative affect in male smokers is therefore consistent with clinical investigations utilizing DHEA as an intervention for depressive symptoms.

Also disclosed herein is the determination that DHEAS levels were inversely correlated with the craving item on the Reasons to Smoke (RTS) Questionnaire, adjusting for age (p=0.03), a finding that was designated as marginally significant. There were possible trends for an inverse relationship between DHEAS levels and the Fagerstrom Test for Nicotine Dependence (FTND) and the Ikard Smoking Motivation Questionnaire (ISMQ) addiction subscale, adjusting for age (p=0.067 and 0.059, respectively). This appears to be the first report to suggest a potential association between DHEAS levels and nicotine dependence severity measures. Since higher DHEAS levels have been observed in smokers compared to non-smokers, it is possible that an upregulation in this neuroactive steroid might be relevant to tobacco addiction. This is consistent with the possibility of a general upregulation in the HPA axis in smokers, since the administration of Adrenocorticotropic Hormone (ACTH; (Rasmusson et al., 2004; Genazzani et al., 1998; Parker, 1999) and Corticotropin-releasing Factor (CRF; Genazzani et al., 1998; Bernardi et al., 2000) increases DHEA levels. Similarly, ALLO levels also increase after administration of both ACTH (Genazzani et al., 1998) and CRF (Genazzani et al., 1998; Bernardi et al., 2000).

Since initial evidence suggests that decreases in cortisol might be relevant to withdrawal symptoms following smoking cessation (Frederick et al., 1998) and might impact relapse likelihood (al'Absi et al., 2004), possible inverse correlations between serum DHEAS levels and measures of nicotine dependence severity suggest that DHEAS might also be relevant to HPA axis interactions with smoking cessation. Since DHEA levels appear to decrease following smoking cessation (Oncken et al., 2002), changes in DHEA or its sulfated derivative DHEAS might also predict withdrawal symptoms or relapse risk.

Given data demonstrating an inverse relationship between serum DHEAS levels and negative affect and possibly nicotine dependence severity measures in male smokers disclosed herein, DHEA might represent a logical candidate as a potential smoking cessation agent. Administering DHEA results in elevated DHEAS levels (Schmidt et al., 2005; Strous et al., 2003; Genazzani et al., 2003). Given the DHEAS findings disclosed herein, it is therefore possible that increasing DHEAS by administering DHEA could represent a multi-pronged approach to smoking cessation. Specifically, it could potentially decrease craving for cigarettes and also attenuate negative affect during withdrawal, therefore theoretically reducing relapse likelihood via two distinct mechanisms. Given the high prevalence of depression among smokers and high rates of affective symptoms during nicotine cessation, short-term DHEA administration might represent a strategy to decrease the emergence of depressive symptoms during smoking abstinence while also impacting craving.

Data presented herein suggested that serum ALLO levels were positively correlated with salivary cotinine levels in male smokers, suggesting that smokers with the greatest degree of nicotine intake might have upregulated ALLO levels compared to smokers with more modest nicotine consumption. This finding remained significant even when a more conservative p value≦0.01 was applied. A correlation between ALLO levels and salivary nicotine levels was not demonstrated, however, suggesting that ALLO changes might be somewhat more chronic in nature (since cotinine has a longer half-life compared to nicotine), although it remains possible that this initial study might have been underpowered to detect a potential correlation between these two variables. In addition, nicotine saliva levels might demonstrate a greater degree of variability and correlate less strongly with nicotine plasma levels in smokers (Shin et al., 2002), in contrast to strong correlations generally observed between saliva cotinine and plasma cotinine levels (Jarvis et al., 2003).

Since ALLO increases after a number of acute stressors in rodent models (Purdy et al., 1991; Morrow et al., 1995; Barbaccia et al., 1996; Barbaccia et al., 1998; Vallee et al., 2000) and also appears to increase following stress in clinical populations (Girdler et al., 2001), ALLO correlations with cotinine levels might be consistent with a general HPA axis activation that involves other steroids in addition to ALLO, including DHEAS, ANDRO, and cortisol (the latter three steroids are increased in smokers).

Since ALLO has pronounced anxiolytic activity in a number of animal models (Crawley et al., 1986; Wieland et al., 1991; Brot et al., 1997), it is also possible that potential ALLO elevations after smoking could contribute to the anxiolytic-like and stress-reducing effects of smoking frequently described by subjects with nicotine dependence. Nicotine administration at very high doses can be anxiogenic, however, and therefore the precise functional significance of dose-dependent nicotine-induced elevations in ALLO in rodent brain remains to be elucidated (Porcu et al., 2003).

Potentially relevant to negative affect during smoking withdrawal, decreased ALLO levels have been associated with chronic social isolation stress in animal models (Dong et al., 2001; Pinna et al., 2003) and depressive symptoms in humans (Uzunova et al., 1998). Although this possibility remains to be tested, the data disclosed herein suggest that ALLO levels might be decreased with longer-term smoking cessation, and that decreases in this GABAergic neuroactive steroid could thus contribute to negative affect, depressive symptoms, and anxiety symptoms frequently reported during nicotine withdrawal.

A possible trend was also observed correlating PG levels with salivary cotinine levels, again potentially suggesting an upregulation in HPA axis activity with greater nicotine intake. Since PG is a potential precursor for many steroids, including ALLO, DHEAS, ANDRO, and cortisol, it is logical that this precursor molecule might also be upregulated in smokers

Many steroids are disclosed herein to be highly interrelated, suggesting that it might be important to analyze biological samples (e.g., from subjects with one or more conditions of interest) for multiple steroids in order to understand their metabolism profiles more fully. The large number of significant correlations among steroids disclosed herein also implies that higher DHEAS, ANDRO, and cortisol levels observed in smokers compared to nonsmokers in prior investigations might be consistent with a general upregulation in several steroid biosynthetic pathways relevant to HPA axis activity. It is also possible that an abrupt change in these steroid metabolism profiles after smoking cessation might contribute to symptoms of nicotine withdrawal and modulate affective symptomatology.

Neuroactive Steroids and other Conditions. Based on the observations in schizophrenia subjects, subjects with AD, and smokers, other conditions appear to be good candidates for intervention by neuroactive steroid therapies. For example, PG and PG metabolites such as ALLO might be involved in depressive symptomatology. Fluoxetine has been shown to elevate ALLO levels in brain and also elevates PG levels (Marx et al., 2006d). In addition, it has been determined that ALLO predicted depressive symptoms as measured by the Beck Depression Inventory and the SCL-90 depression component.

Additionally, neuroactive steroid therapy might be beneficial in subjects with PTSD. Increasing clinical evidence supports a potential role for neurosteroids in the treatment of PTSD and other disorders in which anxiety symptoms are present. Specifically, a recent clinical.trial determined that augmentation with the neurosteroid DHEA decreased anxiety and depression symptoms in patients with schizophrenia (Strous et al., 2003). Since evidence suggests that DHEA administration can increase PG levels in humans (Roberts, 1995), it is possible that DHEA-induced increases in PG levels might have contributed to the efficacy of DHEA augmentation in the Strous et al. study.

Roberts, 1995 suggested that DHEA increases PG levels by inhibiting the conversion of PG to its metabolite 17α-OH-PG. Conversely, PG administration might increase DHEA levels (Morley et al., 1997), possibly by the following biosynthetic pathway:

PG→17α-OH-PG→DHEA

It is therefore possible that direct administration of PG would be at least as efficacious as

DHEA augmentation for anxiety symptoms.

It has also been demonstrated that DHEA administration in humans (50 mg) increases the GABAergic neurosteroid ALLO three-fold (Stomati et al., 2000). Thus, DHEA-induced ALLO elevations might also have played a role in the efficacy of DHEA augmentation for anxiety symptoms in the trial by Strous et al., which is potentially interesting since ALLO is elevated following the administration of certain antipsychotics (Marx et al., 2000; Marx et al., 2003), and demonstrates pronounced anxiolytic, antidepressant, anticonvulsant, and antidopaminergic effects. The administration of a pregnenolone precursor (e.g., DHEA) might also increase ALLO by the following pathway (in addition to increasing DHEA levels): PG to progesterone to 5α-dihydroprogesterone to ALLO. It can therefore be relevant to measure levels of PG and PG metabolites such as DHEA and ALLO to identify specific steroid alterations that can indicate clinical efficacy in the course of PG augmentation.

A substantial rodent literature also demonstrates that PG (or its sulfated derivative) markedly enhances memory performance (Flood et al., 1992; Flood et al., 1995; Vallee et al., 1997; Akwa et al., 2001; Vallee et al., 2001). In one of these studies, levels of pregnenolone sulfate (PGS) in the hippocampus of aged rats were positively correlated with cognitive performance, and the administration of PGS directly into the hippocampus of rats with poor cognitive performance transiently corrected this deficit (Vallee et al., 1997). Evidence also suggests that oral PG administration in humans rapidly increases both PG and PGS levels in plasma (Roberts, 1995).

Since individuals with PTSD demonstrate deficits in learning, attention, memory, cognition, and impulsivity in addition to core PTSD symptoms, PG augmentation of selective serotonin reuptake inhibitor (SSRI) treatment-as-usual represents a potential pharmacologic intervention for cognitive symptoms in PTSD. Evidence of impairment in the ventromedial, prefrontal cortex and deficits in decision making has been reported in PTSD. Learning, memory, and attention problems associated with PTSD have also been described (Bremner et al., 1993; Buckley et al., 2000). Notably, studies in cohorts with PTSD have reported abnormalities in sustained attention, recall, selective attention, and executive function (Jenkins et al., 1998; Jenkins et al., 2000).

Since converging preclinical evidence strongly suggests that PG treatment enhances cognitive performance, this neurosteroid is to be tested as an agent that can enhance cognition in patients with PTSD. The randomized, placebo-controlled, double-blind, 8 week study set forth in EXAMPLE 10 tests the therapeutic potential of augmenting a stable SSRI regimen with PG in an attempt to reduce the cognitive symptoms in patients with a DSM-IV diagnosis of PTSD. PG levels are monitored and PG metabolite levels (including DHEA and ALLO) are also determined to investigate if increases in PG or other neurosteroid metabolites are correlated with therapeutic efficacy.

Additional possibilities for therapeutic intervention include traumatic brain injury, (TBI), alcohol and drug use disorders, and nicotine cessation. With respect to TBI, based on the potential neuroprotective effects observed for PG, possibly through metabolism to ALLO, PG administration constitutes a potential precursor loading strategy resulting in elevated ALLO levels that might be clinically therapeutic in TBI subjects. Rodent data suggested that ALLO is elevated following alcohol administration and relevant to its behavioral effects (VanDoren et al., 2000). Since the data disclosed herein suggested that PG administration results in a 5-fold increase in downstream ALLO formation, PG administration constitutes a potential target in the treatment of alcohol use disorders.

### REFERENCES

All references listed below, as well as all references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
Akwa et al. (2001) Proc Natl Acad Sci USA 98:14033-14037.
al'Absi et al. (2002) Pharmacol Biochem Behav 72:707-716.
al'Absi et al. (2003) Pharmacol Biochem Behav 74:401-410.
al'Absi et al. (2004) Drug Alcohol Depend 73:267-278.
Anda et al. (1990) J Am Med Assn 264:1541-1545.
Baldessarini et al. (2006) Bipolar Disorders 8:625-639.
Barbaccia et al. (1996) Neuroendocrinology 63:166-172.
Barbaccia et al. (1998) Exp Gerontol 33:697-712.
Barbaccia et al. (2001) Neuropsychopharmacology 25:489-497.
Baron et al. (1995) J Pharmacol Exp Ther 272:151-155.
Barrett-Connor et al. (1986) N Engl J Med 315:1519-1524.
Belelli & Lambert (2005) Nature Reviews Neuroscience 6:565-575.
Berkow et al. (1997) The Merck Manual of Medical Information, Home ed., Merck Research Laboratories, Whitehouse Station, New Jersey.
Bernardi et al. (2000) Eur J Endocrinol 142:466-471.
Bjornerem et al., (2004) J Clin Endocrinol Metab 89:6039-6047.
Bradford (2006) “Pregnenolone alterations in parietal cortex are associated with death by suicide in patients with schizophrenia” American College of Neuropsychopharmacology Annual Meeting, Dec. 3-7, 2006, Hollywood, Florida.
Braak & Braak (1991) Acta Neuropathol (Berl) 82:239-259.
Bremner et al. (1993) Am J Psychiatry 150:1015-1019.
Breslau et al. (1993) Arch Gen Psychiatry 50:31-35.
Brot et al. (1997) Eur J Pharmacol 325:1-7.
Buckley et al. (2000) Clin Psychol Rev 20:1041-1065.
Caggiula et al. (1998) Psychoneuroendocrinology 23:143-159.
Charalampopoulos et al. (2004) Proc Natl Acad Sci USA 101:8209-8214.
Chen et al. (1999) J Neurochem 72:879-882.
Chen et al. (2000) J Neurochem 75:1729-1734
Chuang (2005) Ann NY Acad Sci 1053:195-204.
Compagnone & Mellon (1998) Proc Natl Acad Sci USA 95:4678-4683.
Covey et al. (1997) Am J Psychiatry 154:263-265.
Crawley et al. (1986) Brain Res 398:382-385.
Darnaudery et al., (1998) J Neurochem 71:2018-2022.
Darnaudery et al. (2002) Brain Res 951:237-242.
Debonnel et al. (1996) J Endocrinol 150:S33-S42.
Djebaili et al. (2005) J Neurotrauma 22:106-118.
Dong et al. (2001) Proc Natl Acad Sci USA 98:2849-2854.
Duch et al. (1998) Toxicol Lett 100-101:255-263.
Ebadi (1998) CRC Desk Reference of Clinical Pharmacology. CRC Press, Boca Raton, Fla.
Einat et al. (2005) Behavioral Brain Res 165:172-180.
Fagerstrom (1978) Addict Behavior 3:235-241.
Feldman et al. (1998) Ann Epidemiol 8:217-228.
Field et al. (1994) J Clin Endocrinol Metab 79:1310-1316.
Fontaine-Lenoir et al. (2006) Proc Natl Acad Sci US A 103:4711-4716.
Flood et al. (1992) Proc Natl Acad Sci USA 89:1567-1571.
Flood et al. (1995) Proc Natl Acad Sci USA 92:10806-10810.
Frederick et al. (1998) Biol Psychiatry 43:525-530.
Freireich et al. (1966) Cancer Chemother Rep 50:219-244.
Gawande (2004) N Engl J Med 351:2471-2475.
Genazzani et al. (1998) J Clin Endocrinol Metab 83:2099-2103
George et al. (1994) Biological Psychiatry 35:775-780.
Gilbert et al. (1999) Exp Clin Psychopharmacol 7:427-443.
Girdler et al. (2001) Biol Psychiatry 49:788-797.
Gironda et al. (2006) Pain Med 7:339-343.
Glassman et al. (1990) J Am Med Assn 264:1546-1549.
Glassman et al. (2001) Lancet 357:1929-1932.
Goff & Coyle (2001) Am J Psychiatry 158:1367-1377.
Goff et al. (1999) Arch Gen Psychiatry 56:21-27.
Goodman et al. (1996) Goodman & Gilman's the Pharmacological Basis of Therapeutics, 9th ed., McGraw-Hill Health Professions Division, New York.
Gossain et al. (1986) Am J Med Sci 291:325-327.
Gray et al. (2003) J Clin Psychiatry 64 Suppl 5:3-17.
Green (1996) Am J Psychiatry 153:321-330.
Griffin et al. (2004) Nature Med 10:704-711.
Hautanen et al. (1993) J Steroid Biochem Mol Biol 46:245-251.
Hennion et al. (2002) Bipolar Disorders 4:201-206.
Heresco-Levy et al. (1999) Arch Gen Psychiatry 56:29-36.
Hoge et al. (2004) N Engl J Med 351:13-22.
Hoge et al. (2006) J Am Med Assn 295:1023-1032.
Hsu et al. (2006) Nature 439:480-483.
Hughes et al. (1988) Biol Psychiatry 23:96-98.
Ikard et al. (1969) Int J Addict 4:649-659.
Imamura & Prasad (1998) Biochem Biophys Res Commun 243:771-775.
Irwin et al., (1994) J Pharmacol Exp Ther 271:677-682.
Jacob et al. (1981) J Chromatogr 222:61-70.
Jarvis et al. (2003) Nicotine Tob Res 5:349-355.
Javitt (2004) Mol Psychiatry 9:984-997.
Jenkins et al., (1998) Am J Psychiatry 155:278-279.
Jenkins et al. (2000) Clin Neuropsychol 14:7-12.
Jin et al. (2005) Neuropharmacology 48:576-583.
Kassel et al. (2003) Psychol Bull 129:270-304.
Katzung (2001) Basic & Clinical Pharmacology, 8th ed., Lange Medical Books/McGraw-Hill Medical Pub. Division, New York.
Kendler et al. (1993) Arch Gen Psychiatry 50:36-43.
Khaw et al. (1988) N Engl J Med 318:1705-1709.
Kokate et al. (1996) Neuropharmacology 35:1049-1056.
Krystal et al. (1994) Arch Gen Psychiatry 51:199-214.
Lahti et al. (1995) Neuropsychopharmacology 13:9-19.
Lahti at al. (2001) Neuropsychopharmacology 25:455-467.
Laughlin & Barrett-Connor (2000) J Clin Endocrinol Metab 85:3561-3568.
Law at aL (1997) Eur J Epidemiol 13:553-558.
Majewska et al., (1988) Science 232:1004-1007.
Malhotra et al. (1997) Neuropsychopharmacology 17:141-150.
Marx et al. (2000) Biol Psychiatry 47:1000-1004.
Marx et al. (2003) Neuropsychopharmacology 28:1-13.
Marx et al., (2006a) Pharmacol Biochem Behavior 84:598-608.
Marx et al. (2006b) Neuropsychopharmacology 31:1249-1263.
Marx et al. (2006c) Biol Psychiatry 60:1287-1294.
Marx et al. (2006d) Pharmacol Biochem Behavior 84:609-617.
Matta et al. (1998) Psychoneuroendocrinology 23:103-113
Meliska et al. (1995) J Allergy Clin Immunol 95:901-910.
Meltzer et al. (2003) Arch Gen Psychiatry 60:82-91.
Millan (2005) Psychopharmacology 179:30-53.
Mora et al. (1999) Eur J Biochem 266:886-891.
Mora et al. (2002) Bipolar Disorders 4:195-200.
Morley et al. (1997) Proc Natl Acad Sci USA 7537-7542.
Morrow et al. (1987) Eur J Pharmacol 142:483-485.
Morrow et al. (1995) Ann NY Acad Sci 771:257-272.
Oakley et al. (2001) Aust N Z Journal of Psychiatry 35:833-840.
Oncken et al. (2002) Nicotine Tob Res 4:451-458.
Palmer et al. (2005) Arch Gen Psychiatry 62(3):247-53.
Paperwalla et al. (2004) Med Clin North Am 88:1483-1494, x-xi.
Park-Chung at al. (1999) Brain Res 830:72-87.
Parker (1999) Steroids 64:640-647.
Paul & Purdy (1992) FASEB J 6:2311-2322.
PCT International Publication No. WO 93/25521.
Pickworth & Fant (1998) Psychoneuroendocrinology 23:131-141.
Pickworth et al. (1996) Pharmacol Biochem Behav 55:433-437.
Pinna et al. (2003) Proc Natl Acad Sci USA 100:2035-2040.
Pomerleau et al. (2001) J Addict Dis 20:73-80.
Porcu et al. (2003) Pharmacol Biochem Behav 74:683-690.
Puddey et al. (1984) Clin Exp Pharmacol Physiol 11:423-426.
Purdy et al. (1991) Proc Natl Acad Sci USA 88:4553-4557.
Quattrocki et al. (2000) Hary Rev Psychiatry 8:99-110.
Rasmusson et al. (2004) Neuropsychopharmacology 29:1546-1557.
Rasmusson et al. (2006) Biol Psychiatry 60:704-713.
Remington et al. (1975) Remington's Pharmaceutical Sciences, 15th ed., Mack Pub. Co., Easton, Pa.
Robert (1995) Biochem Pharmacol 49:1-16.
Rose et al. (2003) Pharmacol Biochem Behav 76:307-313.
Rosecrans & Karin (1998) Psychoneuroendocrinology 23:95-102.
Rupprecht & Holsboer (1999) Trends Neurosci 22:410-416.
Salvini et al. (1992) J Clin Endocrinol Metab 74:139-143.
Saunders et al. (1993) Neurology 43:1467-1472.
Schmidt et al. (2005) Arch Gen Psychiatry 62:154-162.
Schule et al. (2007) Psychoneuroendocrinology 32:669-680.
Seal et al. (2007) Arch Intern Med 167:476-482.
Shiffman & Jarvik (1976) Psychopharmacol (Berl) 50:35-39.
Shin et al. (2002) J Chromatogr B Analyt Technol Biomed Life Sci 769:177-183.
Sinha (2005) in Steckler et al. (eds) Handbook of Stress and the Brain, Elsevier Science, Amsterdam, the Netherlands, pp 333-356.
Speight et al. (1997) Avery's Drug Treatment: A Guide to the Properties, Choice, Therapeutic Use and Economic Value of Drugs in Disease Management, 4th ed. Adis International, Auckland/Philadelphia.
Stein et al. (2002) Am J Psychiatry 159:1777-1779
Strittmatter et al. (1993) Proc Natl Acad Sci USA 90:1977-1981.
Stomati et al. (2000) Gynecol Endocrinol 14:342-363.
Strous et al. (2003) Arch Gent Psychiatry 60:133-141.
Strous et al. (2004) Schizophr Res 71:427-434.
Tziomalos & Charsoulis (2004) Clin Endocrinol (Oxf) 61:664-674.
U.S. Patent Application Publication No. 20060188558.
U.S. Pat. Nos. 3,598,122; 5,016,652; 5,326,902; 5,234,933; 5,935,975; 6,106,856; 6,162,459; 6,180,082; 6,495,605; and 6,582,724.
Uzunova et al. (1998) Proc Natl Acad Sci USA 95:3239-44.
Vallee et al. (1997) Proc Natl Acad Sci US A 94:14865-14870.
Vallee et al. (2000) Anal Biochem 287:153-166.
Vallee et al., (2001) Brain Res Brain Res Rev 87:5138-5143.
Van Doren et al. (2000) J Neurosci 20:1982-1989.
Wang et al. (2005) J Neurosci 25:4706-4718.
Wieland et al. (1991) Brain Res 565:263-268.
Wolkowitz et al. (1999) Am J Psychiatry 156:646-649.
Wu et al. (1991) Mol Pharmacol 40:333-336.
Xilouri & Papazafiri (2006) Eur J Neurosci 23:43-54.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for ameliorating a symptom of a pain disorder, a chronic pain disorder, or both in a subject, the method comprising administering to the subject an effective amount of a neuroactive steroid composition comprising pregnenolone (PG), a pharmaceutically acceptable salt thereof, a derivative thereof, or a combination thereof.

2. The method of claim 1, wherein the neuroactive steroid composition is administered in a sustained release formulation, a controlled release formulation, or a combination thereof.

3. The method of claim 2, wherein the sustained release formulation, the controlled release formulation, or the combination thereof is selected from the group consisting of an oral formulation, a peroral formulation, a buccal formulation, an enteral formulation, a pulmonary formulation, a rectal formulation, a vaginal formulation, a nasal formulation, a lingual formulation, a sublingual formulation, an intravenous formulation, an intraarterial formulation, an intracardial formulation, an intramuscular formulation, an intraperitoneal formulation, a transdermal formulation, an intracranial formulation, an intracutaneous formulation, a subcutaneous formulation, an aerosolized formulation, an ocular formulation, an implantable formulation, a depot injection formulation, and combinations thereof.

4. The method of claim 1, wherein the neuroactive steroid composition comprises at least two active agents selected from the group consisting of PG, allopregnanolone (ALLO), dehydroepiandrosterone (DHEA), pharmaceutically acceptable salts thereof, and derivatives thereof.

5. The method of claim 1, wherein the effective amount is sufficient to raise the level of PG, ALLO, DHEA, or combinations thereof in a source selected from the group consisting of cerebrospinal fluid, serum, plasma, blood, saliva, skin, muscle, olfactory tissue, lacrimal fluid, synovial fluid, nail tissue, hair, feces, urine, in the subject by at least 1.5-fold within 8 weeks from a level in the source in the subject prior to the administering step.

6. The method of claim 1, wherein the effective amount comprises a daily dose ranging from about 0.005 mg to about 2000 mg of PG, or an equivalent molar amount of the pharmaceutically acceptable salt thereof, the derivative thereof, or the combination thereof.

7. The method of claim 1, wherein the derivative is pregnenolone sulfate (PGS).

8. The method of claim 1, wherein in addition to pain, the subject has a psychological symptom selected from the group consisting of depression, irritability, agitation, difficulty concentrating, tension, anger, stress, anxiety, and combinations thereof.

9. The method of claim 8, further comprising administering to the subject at least one additional composition selected from the group consisting of an antidepressant, an anxiolytic, an antipsychotic, an anticonvulsant, and a mood stabilizer, wherein the at least one additional composition is administered to the subject before, after, at the same time as, or a combination thereof the neuroactive steroid composition.

10. The method of claim 9, wherein the at least one additional composition is administered to the subject in a daily dose of at least about 0.005 mg.

11. The method of claim 1, wherein the effective amount is sufficient to improve a cognitive function in the subject.

12. The method of claim 11, further comprising testing the subject for an improvement in cognitive functioning by employing a cognitive test.


SEQUENCE LISTING (4 sequences)

SEQ ID NO: 1 - DNA, 20 bp, Artificial Sequence
  Artificially synthesized oligonucleotide primer
cttgggtggagaggctattc

SEQ ID NO: 2 - DNA, 20 bp, Artificial Sequence
  Artificially synthesized oligonucleotide primer
aggtgagatgacaggagatc

SEQ ID NO: 3 - DNA, 30 bp, Artificial Sequence
  Artificially synthesized oligonucleotide primer
ctttgtggaactgtacggccccagcatgcg

SEQ ID NO: 4 - DNA, 30 bp, Artificial Sequence
  Artificially synthesized oligonucleotide primer
acagcctgcagctttgtttcatggtacatc